(19) United States
(12) Patent Application Publication
(10) Pub. No.: US 2024/0350410 A1
(43) Pub. Date: Oct. 24, 2024

(54) FREEZE-DRYING OF LIPID NANOPARTICLES (LNPS) ENCAPSULATING RNA AND FORMULATIONS THEREOF

(71) Applicant: GLAXOSMITHKLINE BIOLOGICALS SA, Rixensart (BE)

(72) Inventors: Rushit LODAYA, Rockville, MD (US); Patrick POHLHAUS, Rockville, MD (US); Elizabeth ZECCA, Rockville, MD (US); Jinjin ZHANG, Rockville, MD (US); Adora PADILLA, Rockville, MD (US); Erwan BOURLÈS, Rixensart (BE); Julie ENERT, Rixensart (BE)

(73) Assignee: GLAXOSMITHKLINE BIOLOGICALS SA, Rixensart (BE)

(21) Appl. No.: 18/683,535

(22) PCT Filed: Aug. 16, 2022
(86) PCT No.: PCT/IB2022/057683

§ 371 Date: Feb. 14, 2024

Related U.S. Application Data
(60) Provisional application No. 63/233,517, filed on Aug. 16, 2021.

(51) Int. Cl. A61K 9/127; A61K 9/19; A61K 31/7105; A61K 47/18; A61K 47/22; A61K 47/26

(52) U.S. Cl. CPC A61K 9/1272; A61K 9/1271; A61K 9/19; A61K 31/7105; A61K 47/183; A61K 47/22; A61K 47/26

(57) ABSTRACT

Compositions and methods are provided for stabilization of RNA encapsulated by lipid nanoparticles during lyophilization. Novel lyophilization processes are provided. These techniques may be used to prevent the need for cold chain storage.

### CROSS-REFERENCE TO RELATED APPLICATION

This application claims priority to U.S. Provisional Application No. 63/233,517 filed on Aug. 16, 2022, the entirety of which is incorporated by reference herein.

### TECHNICAL FIELD

The present invention relates to the field of formulations and methods of lyophilization, and in particular, to pharmaceutical compositions for lyophilization of RNA encapsulated in a lipid nanoparticle and methods thereof. The present invention provides a pharmaceutical composition comprising a lipid nanoparticle formulated for lyophilization, a vaccine, a kit, and a method of lyophilization of the pharmaceutical composition. The present invention provides novel pharmaceutical compositions formulated for lyophilization, including a lipid nanoparticle encapsulating RNA, a primary sugar, and a buffer with one or more optional additional components. The present invention provides a novel method of lyophilization of the pharmaceutical composition, wherein the method comprises a freezing step, a primary drying step, and a secondary drying step. The present invention provides a novel use of a pharmaceutical composition for stabilizing lipid nanoparticle encapsulated RNA during lyophilization. The present invention provides a novel use of the inventive pharmaceutical composition and/or method in the manufacture of a medicament, a vaccine, and/or kit.

### BACKGROUND

A major challenge for mRNA vaccines is efficient delivery to the target site while avoiding degradation and clearance from the body. To overcome this barrier, lipid nanoparticles (LNPs), in which RNA is encapsulated in a liquid core of a LNP, have emerged as one of the most efficient delivery vehicles for RNA (see EP2591103). The LNP protects the RNA from degradation and enables its cellular uptake and intracellular release.

However, a disadvantage of lipid nanoparticle encapsulated RNA (RNA-LNP) is its instability, (e.g., due to susceptibility to hydrolysis). To avoid such degradation, RNA-LNP is typically stored at minus 20° C. to minus 80° C. under RNase free conditions.

For distribution of RNA-LNP containing therapeutics, the pharmaceutical composition containing the RNA-LNP is subject to cold chain storage, in which it is typically maintained at a temperature ranging from −20° C. to −80° C. during its lifecycle. However, maintaining cold chain storage has a number of disadvantages, including cost, complexity of distribution, and potential loss of product. In some cases, failure to maintain cold chain storage, e.g., due to refrigeration failure, electrical outages, etc., may necessitate discarding of the pharmaceutical composition. Additionally, once reaching respective endpoints, pharmaceutical compositions that cannot be stored at the designated temperature must be administered within a prescribed timeframe or discarded.

Accordingly, it is desirable to improve long term stability of pharmaceutical compositions comprising RNA as well as prevent and/or reduce the need for cold chain storage, with the goal to preserve RNA entrapment in lipid nanoparticles while maintaining transfection potency after lyophilization.

### BRIEF SUMMARY

The invention provides a pharmaceutical composition formulated for lyophilization of lipid nanoparticle encapsulated RNA (RNA-LNP), along with kits and vaccines. The pharmaceutical composition formulated for stable lyophilization of lipid nanoparticle encapsulated RNA may comprise lipid nanoparticle encapsulated RNA (RNA-LNPs) at a concentration ranging from about 60 μg/mL to 250 μg/mL, wherein the lipid nanoparticle comprises a cationic lipid, a zwitterionic lipid, a cholesterol, and a PEG, and wherein the pharmaceutical composition comprises sucrose in an amount of at least 5% (w/v) and a buffer.

In certain embodiments, the pharmaceutical composition is designed to stabilize the LNP encapsulated RNA during lyophilization. The invention also provides a novel method of lyophilization of the pharmaceutical compositions provided herein. In certain embodiments, the lyophilization process comprises an initial freezing step, a primary drying step, and a secondary drying step. The pharmaceutical composition is designed to maintain the stability of the LNP encapsulated RNA during the lyophilization process and allow storage under standard refrigeration temperatures or room temperature. In certain embodiments, the invention also provides for lyophilized compositions made from the pharmaceutical composition, as well as reconstituted vaccine compositions made from the lyophilized compositions. In certain embodiments, the invention also relates pharmaceutical composition for use in inducing an immune response in a subject where the pharmaceutical composition is administered by injection. In certain embodiments, the invention is also directed to the manufacture of a medicament for inducing an immune response in a subject, wherein the medicament is prepared to be administered by injection.

### BRIEF DESCRIPTION OF DRAWINGS

FIG. 1 shows percent encapsulation of various pharmaceutical compositions comprising different primary sugars at a concentration of 7.5% (w/v).

FIG. 2A shows percent encapsulation of various pharmaceutical compositions comprising sucrose at a concentration of about 7.5% (w/v) and amino acids at a concentration of about 0.5% (w/v).

FIG. 2B shows entrapment efficiency of various pharmaceutical compositions comprising sucrose at a concentration of about 5.0% (w/v), a secondary sugar at a concentration of about 2.5% (w/v), and various amino acids at a concentration of about 0.5% (w/v).

FIG. 2C shows entrapment efficiency of various pharmaceutical compositions comprising sucrose at a concentration of about 5.0% (w/v), a secondary sugar at a concentration of about 2.5% (w/v), and various amino acids at a concentration of about 0.5% (w/v).

FIGS. 3A and 3B show heat maps for percentage encapsulation for various pharmaceutical compositions, which include a plasticizer of either sorbitol or glycerol, with amino acid(s) and secondary sugar(s) listed on different axes. In these heat maps, the warmer (red) colors (corresponding to higher % encapsulation) are higher numbers and the colder (blue) colors (corresponding to lower % encapsulation) are lower numbers.

FIG. 4 shows a Design of Experiments (DoE) study with pharmaceutical compositions comprising SAM-LNP, characterized by an overall LNP size of 112 nm and a PDI of 0.135 with 89% encapsulation.

FIG. 5 shows percent encapsulation for compositions comprising about 60-250 ug/mL SAM-LNP and 7.5% (w/v) sucrose with varying concentrations of citrate buffer (e.g., 5 mM, 10 mM and 20 mM) at pH 6.0.

FIG. 6A shows percent encapsulation for compositions comprising about 60-250 ug/ml SAM-LNP and 7.5% (w/v) sucrose with varying concentrations of histidine buffer (e.g., 5 mM, 10 mM and 20 mM) at various pHs (e.g., 6.0 and 6.5).

FIG. 6B shows sizes (nm) of reconstituted compositions comprising mRNA-LNP that underwent lyophilization.

FIG. 7 shows percent encapsulation for compositions comprising about 60-250 ug/ml SAM-LNP and 7.5% (w/v) sucrose with varying concentrations of Tris buffer (5 mM, 10 mM, and 20 mM at various pHs ranging from 7.5 to 9.0).

FIG. 8 shows percent encapsulation of compositions comprising HEPES buffer that underwent lyophilization.

FIG. 9A shows percent encapsulation after reconstitution of a lyophilized pharmaceutical composition comprising higher sucrose concentrations (up to about 20% sucrose).

FIG. 9B shows the size and PDI of the reconstituted LNPs (as described in FIG. 9A) as the amount of sucrose was increased in the formulation.

FIG. 10 shows that the addition of a plasticizer, in this case glycerol, improved the percentage entrapment of RNA-LNP after lyophilization.

FIG. 11A shows that the addition of another plasticizer, in this case sorbitol, also improved the percentage entrapment of SAM-LNP after lyophilization.

FIG. 11B shows that the size of the LNPs after lyophilization was not significantly impacted.

FIG. 12 shows an illustration of a lyophilization process comprising an initial freezing step followed by a primary drying step in which a sublimation process removes unbound water from the composition. A primary drying step is followed by a secondary drying step in which bound water molecules are removed. At the end of secondary drying step, the container is filled with inert gas and stoppered.

FIGS. 13A-13C show the impact of freezing ramp rate on the lipid nanoparticle. FIG. 13A shows that the lipid content remained steady during the lyophilization process. FIG. 13B shows that the LNP retain their in vitro potency over a ramp rate ranging from about 0.1° C./min to about 1° C./min. FIG. 13C shows the effects of particle size, percent encapsulation, and RNA content for LNPs over a ramp rate ranging from about 0.1° C./min to about 1° C./min.

FIG. 14 shows data relating to an alternative lyophilization process comprising a secondary drying temperature at 5° C. for 48 hours.

FIG. 15 shows the effect of various excipients added to the control formulation.

FIG. 16 shows in vivo formulations and results demonstrating efficacy of the reconstituted lyophilized compositions.

FIG. 17A is a DSC analysis graph of SAM-LNP samples that have a concentration of 60-400 μg/mL and FIG. 17B is an exploded view including the circled region in FIG. 17A. The heat flow values (on the Y axis) between −5.2 and −6.2 are exploded (expanded) from the scale used in FIG. 17A in order to show the transition at −52.5° C. in FIG. 17A which is not easily observed at the scale shown in FIG. 17A.

### DETAILED DESCRIPTION

The pharmaceutical compositions provided herein stabilize RNA-LNP encapsulation during lyophilization to preserve RNA entrapment in lipid nanoparticles while maintaining transfection potency after lyophilization. These compositions and methods improve long term stability of pharmaceutical compositions comprising RNA as well as prevent or reduce the need for cold chain storage.

The invention provides one or more pharmaceutical compositions formulated for lyophilization of lipid nanoparticle encapsulated RNA (RNA-LNP), along with kits and vaccines. The invention also provides a method of lyophilization of the pharmaceutical composition. In aspects, the lyophilization process comprises an initial freezing step, a primary drying step, and a secondary drying step. The formulation is designed to maintain the stability of the LNP encapsulated RNA during the lyophilization process and allow storage under standard refrigeration temperatures or even at room temperature.

In certain embodiments, the RNA-LNP comprises an mRNA encapsulated in a lipid nanoparticle (LNP). In certain embodiments, the mRNA is a self-amplifying mRNA (SAM) encapsulated in a lipid nanoparticle (LNP) forming a SAM-LNP. In other aspects, the mRNA comprises a non-replicating mRNA. The mRNA may encode one or more antigens, as described in additional detail below.

### The RNA

Any type of RNA that encodes an immunogen or antigen is contemplated for use in accordance with the present invention. The RNA can be a self-replicating mRNA such as SAM or can be a non-replicating mRNA. Any suitable concentration of RNA may be used herein. In aspects, the concentration of SAM-LNP ranges from 60 to 250 μg of SAM-LNP per mL (the pre-lyophilization concentration of SAM-LNP). In aspects, the concentration of SAM-LNP may be about 60 μg of SAM-LNP per mL or more, such as about 60-250 μg/mL, such as about 60-220 ug/mL, about 60-180 ug/mL, about 60-150 μg/mL, about 60-120 ug/mL, about 60-90 ug/mL or preferably about 60 μg/mL, about 90 μg/mL, about 120 μg/mL, about 150 μg/mL, about 180 μg/mL or about 200 μg/mL.

mRNA

Messenger RNA (mRNA) can direct the cellular machinery of a subject to produce proteins. mRNA may be circular or branched, but will generally be linear.

mRNA as used herein are preferably provided in purified or substantially purified form, i.e. substantially free from proteins (e.g., enzymes), other nucleic acids (e.g. DNA and nucleoside phosphate monomers), and the like, generally being at least about 50% pure (by weight), and usually at least 90% pure, such as at least 95%, 96%, 97% or at least 98% pure.

mRNA may be prepared in many ways, e.g., by chemical synthesis in whole or in part, by digesting longer nucleic acids using nucleases (e.g., restriction enzymes), by joining shorter nucleic acids or nucleotides (e.g., using ligases or polymerases), from genomic or cDNA libraries, etc. In particular, mRNA may be prepared enzymatically using a DNA template.

The term mRNA as used herein includes conventional mRNA or mRNA analogs, such as those containing modified backbones or modified bases (e.g., pseudouridine, or the like). mRNA, may or may not have a 5′ cap.

The mRNA comprises a sequence which encodes at least one antigen or immunogen. Typically, the nucleic acids of the invention will be in recombinant form, i.e. a form which does not occur in nature. For example, the mRNA may comprise a sequence encoding an antigen and/or a control sequence such as a promoter or an internal ribosome entry site (IRES). In another example, the mRNA may comprise one or more heterologous nucleic acid sequences (e.g., another sequence encoding another antigen and/or another control sequence such as a promoter or an IRES in addition to the sequence encoding the antigen).

Alternatively, or in addition, the sequence or chemical structure of the nucleic acid may be modified compared to a naturally occurring sequence which encodes the antigen. The sequence of the nucleic acid molecule may be modified, e.g., to increase the efficacy of expression or replication of the nucleic acid, or to provide additional stability or resistance to degradation.

mRNA may also be codon optimised. In some embodiments, mRNA may be codon optimised for expression in human cells. “Codon optimised” is intended to refer to modification(s) with respect to codon usage, which may increase translation efficacy and/or half-life of the nucleic acid.

A poly A tail (e.g., of about 30 adenosine residues or more) may be attached to the 3′ end of the mRNA to increase its half-life.

The mRNA may also include a poly-A polymerase recognition sequence (e.g. AAUAAA) near its 3′ end.

The 5′ end of the mRNA may be capped, for example, with a modified ribonucleotide having the structure m7G (5′) ppp (5′) N (cap 0 structure) or a derivative thereof, which can be incorporated during RNA synthesis or can be enzymatically engineered after RNA transcription (e.g., by using Vaccinia Virus Capping Enzyme (VCE) comprising or consisting of mRNA triphosphatase, guanylyl-transferase and guanine-7-methyltransferase, which catalyzes the construction of N7-monomethylated cap 0 structures). Cap 0 structure plays an important role in maintaining the stability and translational efficacy of the mRNA molecule. The 5′ cap of the mRNA molecule may be further modified by a 2′-O-Methyltransferase which results in the generation of a cap 1 structure (m7Gppp [m2′-O]N), which may further increase translation efficacy.

mRNA may comprise one or more nucleotide analogs or modified nucleotides. As used herein, “nucleotide analog” or “modified nucleotide” refers to a nucleotide that contains one or more chemical modifications (e.g., substitutions) in or on the nitrogenous base of the nucleoside (e.g., cytosine (C), thymine (T) or uracil (U)), adenine (A) or guanine (G)). A nucleotide analog can contain further chemical modifications in or on the sugar moiety of the nucleoside (e.g., ribose, modified ribose, six-membered sugar analog, or open-chain sugar analog), or the phosphate. The preparation of nucleotides and modified nucleotides and nucleosides are well-known in the art (see, U.S. Pat. Nos. 4,373,071, 4,458,066, 4,500,707, 4,668,777, 4,973,679, 5,047,524, 5,132,418, 5,153,319, 5,262,530, 5,700,642). Many modified nucleosides and modified nucleotides are commercially available.

Modified nucleobases (chemical modifications) which can be incorporated into modified nucleosides and nucleotides and be present in the mRNA molecules include: m5C (5-methylcytidine); m5U (5-methyluridine); m6A (N6-methyladenosine); s2U (2-thiouridine); Um (2′-O-methyluridine); m1A (1-methyladenosine); m2A (2-methyladenosine); Am (2-1-O-methyladenosine); ms2m6A (2-methylthio-N6-methyladenosine); i6A (N6-isopentenyladenosine); ms216A (2-methylthio-N6isopentenyladenosine); 106A (N6-(cis-hydroxyisopentenyl) adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosinc); hn6A (N6-hydroxynorvalylcarbamoyl adenosine); ms2hn6A (2-methylthio-N6-hydroxynorvalyl carbamoyladenosine); Ar (p) (2′-O-ribosyladenosine (phosphate)); I (inosine); m′l (1-methylinosine); m′lm (1,2′-O-dimethylinosine); m3C (3-methylcytidine); Cm (2′-O-methylcytidine); s2C (2-thiocytidine); ac4C(N4-acetylcytidine); f5C (5-fonnylcytidine); m5Cm (5,2-O-dimethylcytidine); ac4Cm(N4-acetyl-2-O-methylcytidine); k2C (lysidine); m1G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr (p) (2′-O-ribosylguanosine (phosphate)); yW (wybutosine); 02yW (peroxywybutosine); OHyW (hydroxywybutosine); OHyW*(undermodified hydroxywybutosine); imG (wyosine); mimG(methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G*(archacosine); D (dihydrouridine); m5Um (5,2′-O-dimethyluridine); s4U (4-thiouridinc); m5s2U (5-methyl-2-thiouridinc); s2Um (2-thio-2′-O-methyluridine); acp3U (3-(3-amino-3-carboxypropyl) uridinc); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl) uridine)); mchm5U (5-(carboxyhydroxymethyl) uridine methyl ester); mcm5U (5-methoxycarbonyl methyluridinc); mcm5Um (S-methoxycarbonylmethyl-2-O-methyluridine); mcm5s2U (5-methoxycarbonylmethyl-2-thiouridine); nm5s2U (5-aminomethyl-2-thiouridinc); mnm5U (5-methylaminomethyluridine); mnm5s2U (5-methylaminomethyl-2-thiouridine); mnm5sc2U (5-methylaminomethyl-2-selenouridine); nem5U (5-carbamoylmethyl uridinc); ncm5Um (5-carbamoylmethyl-2′-O-methyluridine); cmnm5U (5-carboxymethylaminomethyluridine); cnmm5Um (5-carboxymethy 1 aminomethyl-2-L-O-methyl uridine); cmnm5s2U (5-carboxymethylaminomethyl-2-thiouridine); m62A (N6,N6-dimethyladenosinc); Im (2′-O-methylinosine); m4C(N4-methylcytidine); m4Cm(N4,2-O-dimethylcytidine); hm5C (5-hydroxymethylcytidine); m3U (3-methyluridinc); cm5U (5-carboxymethyluridine); m6Am(N6,2′-O-dimethyladenosine); rn62Am(N6,N6,0-2-trimethyladenosine); m2′7G (N2,7-dimethylguanosinc); m2′2′7G (N2,N2,7-trimethylguanosine); m3Um (3,2′-O-dimethyluridinc); m5D (5-methyldihydrouridine); f5Cm (5-formyl-2′-O-methylcytidine); m1Gm (1,2′-O-dimethylguanosine); m′Am (1,2-O-dimethyl adenosine) irinomethyluridine); tm5s2U(S-taurinomethyl-2-thiouridine)); iniG-14 (4-demethyl guanosinc); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C1-C6)-alkyluracil, 5-methyluracil, 5-(C2-C6)-alkenyluracil, 5-(C2-C6)-alkynyluracil, 5-(hydroxymethyl) uracil, 5-chlorouracil, 5-fluorouracil, 5-bromouracil, 5-hydroxycytosine, 5-(C1-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. Many of these modified nucleobases and their corresponding ribonucleosides are available from commercial suppliers.

In some embodiments, the modified nucleotides comprise: pseudouridine; N1-methylpseudouridine; N1-ethylpseudouridine; 2-methylthio-N6-(cis-hydroxyisopentenyl) adenosine; 2-methylthio-N6-methyladenosine; 2-methylthio-N6-thrconyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl) adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6,N6 (dimethyl) adenine; N6-cis-hydroxy-isopentenyl-adenosine; alpha-thio-adenosine; 2 (amino) adenine; 2 (aminopropyl) adenine; 2 (methylthio) N6 (isopentenyl) adenine; 2-(alkyl) adenine; 2-(aminoalkyl) adenine; 2-(aminopropyl) adenine; 2-(halo) adenine; 2-(halo) adenine; 2-(propyl) adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl) adenine; 6 (methyl) adenine; 6-(alkyl) adenine; 6-(methyl) adenine; 7 (deaza) adenine; 8 (alkenyl) adenine; 8 (alkynyl) adenine; 8 (amino) adenine; 8 (thioalkyl) adenine; 8-(alkenyl) adenine; 8-(alkyl) adenine; 8-(alkynyl) adenine; 8-(amino) adenine; 8-(halo) adenine; 8-(hydroxyl) adenine; 8-(thioalkyl) adenine; 8-(thiol) adenine; 8-azido-adenosine; aza adenine; deaza adenine; N6 (methyl) adenine; N6-(isopentyl) adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromoadenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; alpha-thio-cytidine; 2-(thio) cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza) cytosine; 3 (methyl) cytosine; 3-(alkyl) cytosine; 3-(deaza) 5 (aza) cytosine; 3-(methyl) cytidine; 4,2′-O-dimethylcytidine; 5 (halo) cytosinc; 5 (methyl) cytosine; 5 (propynyl) cytosine; 5 (trifluoromethyl) cytosinc; 5-(alkyl) cytosine; 5-(alkynyl) cytosine; 5-(halo) cytosine; 5-(propynyl) cytosine; 5-(trifluoromethyl) cytosine: 5-bromo-cytidine; 5-iodo-cytidine; 5-propynyl cytosine; 6-(azo) cytosine; 6-aza-cytidine; aza cytosine; deaza cytosine; N4 (acetyl) cytosine; 1-methyl-1-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl) cytidine TP; 2.2′-anhydro-cytidine TP hydrochloride; 2′Fluor-N4-Bz-cytidine TP; 2′Fluoro-N4-Acetyl-cytidine TP; 2′-O-Methyl-N4-Acctyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl) cytidine TP; 3′-Ethynylcytidine TP; 4′-Azidocytidine TP; 4′-Carbocyclic cytidine TP; 4′-Ethynylcytidine TP; 5-(1-Propynyl) ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosinc; N2,2′-O-dimethylguanosine; N2-methylguanosine; Wyosine; 1,2′-O-dimethylguanosine; 1-methylguanosinc; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archacosine; Methylwyosinc; N2,7-dimethylguanosinc; N2,N2,2′-O-trimethylguanosinc; N2,N2,7-trimethylguanosine; N2,N2-dimethylguanosine; N2,7,2′-O-trimethylguanosine; 6-thio-(propyl) guanine; 2-(alkyl) guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl) guanine; 6-(alkyl) guanine; 6-(methyl) guanine; 6-methyl-guanosinc; 7 (alkyl) guanine; 7 (deaza) guanine; 7 (methyl) guanine; 7-(alkyl) guanine; 7-(deaza) guaninc; 7-(methyl) guanine; 8 (alkyl) guanine; 8 (alkynyl) guanine; 8 (halo) guanine; 8 (thioalkyl) guanine; 8-(alkenyl) guanine; 8-(alkyl) guanine; 8-(alkynyl) guanine; 8-(amino) guanine; 8-(halo) guaninc; 8-(hydroxyl) guanine; 8-(thioalkyl) guanine; 8-(thiol) guanine; aza guanine; deaza guanine; N(methyl) guanine; N-(methyl) guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosinc TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1.2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; (3-(3-amino-3-carboxypropyl) uridine; 1-methyl-3-(3-amino-5-carboxypropyl) pseudouridine; 1-methylpseduouridine; 1-methyl-pseudouridine; 2′-O-methyluridine; 2′-O-methylpseudouridine; 2′-O-methyluridine; 2-thio-2′-O-methyluridine; 3-(3-amino-3-carboxypropyl) uridine; 3,2′-O-dimethyluridine; 3-Methyl-pseudo-Uridine TP; 4-thiouridine; 5-(carboxyhydroxymethyl) uridinc; 5-(carboxyhydroxymethyl) uridine methyl ester, 5,2′-O-dimethyluridine; 5,6-dihydro-uridine; 5-aminomethyl-2-thiouridine; 5-carbamoylmethyl-2′-O-methyluridine; 5-carbamoylmethyluridinc; 5-carboxyhydroxymethyluridine; 5-carboxyhydroxymethyluridine methyl ester. 5-carboxymethylaminomethyl-2′-O-methyluridine; 5-carboxymethylaminomethyl-2-thiouridine; 5-carboxymethylaminomethyl-2-thiouridine; 5-caboxymethylaminomethyluridine; 5-carboxymethylaminomethyluridine; 5-Carbamoylmethyluridine TP; 5-methoxycacoonylmethyl-2′-O-methyluridine; 5-methoxycarbonylmethyl-2-thiouridine; 5-methoxycarbonylmethyluridine; 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine; N1-ethyl-pseudo-uridine; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl) uridine TP; 5-propynyl uracil.alpha-thio-uridine; 1 (aminoalkylamino-carbonylethylenyl)-2 (thio)-pscudouridine; 1 (aminoalkylaminocarbonylethylenyl)-2,4-(dithio) pscudouridinc; 1 (aminoalkylaminocarbonylethylenyl)-4 (thio) pseudouridine; 1 (aminoalkylaminocarbonylethylenyl)-pseudouridinc; 1 (aminocazbonylethylenyl)-2 (thio)-pseudouridine; 1 (aminocarbonylethylenyl)-2.4-(dithio) pseudouridine; 1 (aminocarbonylethylenyl)-4 (thio) pseudouridine; 1 (aminocarbonylethylenyl)-pseudouridine; 1 substituted 2 (thio)-pseudouridine; 1 substituted 2,4-(dithio) pseudouridine; 1 substituted 4 (thio) pseudouridine; 1 substituted pseudouridine; 1-(aminoalkylamino-carbonylethylenyl)-2-(thio)-pseudouridine; 1-Methyl-3-(3-amino-3-carboxypropyl) pseudouridine TP; 1-Methyl-3-(3-amino-3-carboxypropyl) pseudo-UTP; 1-Methyl-pseudo-UTP; 2 (thio) pseudouridine; 2′ deoxy uridine; 2′ fluorouridine; 2-(thio) uracil; 2,4-(dithio) psuedouracil; 2′ methyl, 2′amino, 2′azido, 2′fluoro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl) uracil; 4 (thio) pseudouridinc; 4-(thio) pseudouridine; 4-(thio) uracil; 4-thiouracil; 5 (1,3-diazole-1-alkyl) uracil; 5 (2-aminopropyl) uracil; 5 (aminoalkyl) uracil; 5 (dimethylaminoalkyl) uracil; 5 (guanidiniumalkyl) uracil; 5 (methoxycarbonylmethyl)-2-(thio) uracil; 5 (methoxycarbonylmethyl) uracil; 5 (methyl) 2 (thio) uracil; 5 (methyl) 2,4 (dithio) uracil; 5 (methyl) 4 (thio) uracil; 5 (methylaminomethyl)-2 (thio) uracil; 5 (methylaminomethyl)-2,4 (dithio) uracil; 5 (methylaminomethyl)-4 (thio) uracil; 5 (propynyl) uracil; 5 (trifluoromethyl) uracil; 5-(2-aminopropyl) uracil; 5-(alkyl)-2-(thio) pseudouridine; 5-(alkyl)-2.4 (dithio) pseudouridine; 5-(alkyl)-4 (thio) pseudouridinc; 5-(alkyl) pseudouridine; 5-(alkyl) uracil; 5-(alkynyl) uracil; 5-(allylamino) uracil; 5-(cyanoalkyl) uracil; 5-(dialkylaminoalkyl) uracil; 5-(dimethylaminoalkyl) uracil; 5-(guanidiniumalkyl) uracil; 5-(halo) uracil; 5-(1,3-diazole-1-alkyl) uracil; 5-(methoxy) uracil; 5-(methoxycarbonylmethyl)-2-(thio) uracil; 5-(methoxycarbonylmethyl) uracil; 5-(methyl) 2 (thio) uracil; 5-(methyl) 2,4 (dithio) uracil; 5-(methyl) 4 (thio) uracil; 5-(methyl)-2-(thio) pscudouridinc; 5-(methyl)-2,4 (dithio) pseudouridine; 5-(methyl)-4 (thio) pseudouridine; 5-(methyl) pseudouridine; 5-(methylaminomethyl)-2 (thio) uracil; 5-(methylaminomethyl)-2,4 (dithio) uracil; 5-(methylaminomethyl)-4-(thio) uracil; 5-(propynyl) uracil; 5-(trifluoromethyl) uracil; 5-aminoallyl-uridine; 5-bromo-uridine; 5-iodouridine; 5-uracil; 6 (azo) uracil; 6-(azo) uracil; 6-aza-uridine; allyamino-uracil; aza uracil; deaza uracil; N3 (methyl) uracil; Pseudo-UTP-1-2-cthanoic acid; Pseudouridine; 4-Thio-pseudo-UTP; 1-carboxymethyl-pseudouridine; 1-methyl-1-deaza-pscudouridinc; 1-propynyl-uridinc; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-decaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pscudouridinc; 4-thio-pseudouridine; 5-aza-uridine; Dihydropseudouridine; (.+−.) 1-(2-Hydroxypropyl) pscudouridinc TP; (2R)-1-(2-Hydroxypropyl) pseudouridine TP; (2S)-1-(2-Hydroxypropyl) pseudouridine TP; (E)-5-(2-Bromo-vinyl) ara-uridine TP; (E)-5-(2-Bromo-vinyl) uridinc TP; (Z)-5-(2-Bromo-vinyl) ara-uridine TP; (Z)-5-(2-Bromo-vinyl) uridine TP; 1-(2,2,2-Trifluorocthyl)-pseudo-UTP; 1-(2,2,3,3,3-Pentafluoropropyl) pseudouridine TP; 1-(2,2-Dicthoxyethyl) pseudouridine TP; 1-(2,4,6-Trimethylbenzyl) pseudouridine TP; 1-(2,4,6-Trimethyl-benzyl) pseudo-UTP; 1-(2,4,6-Trimethyl-phenyl) pseudo-UTP; 1-(2-Amino-2-carboxyethyl) pseudo-UTP; 1-(2-Amino-ethyl) pseudo-UTP; 1-(2-Hydroxyethyl) pseudouridine TP; 1-(2-Methoxyethyl) pseudouridine TP; 1-(3,4-Bis-trifluoromethoxybenzyl) pseudouridine TP; 1-(3,4-Dimethoxybenzyl) pseudouridine TP; 1-(3-Amino-3-carboxypropyl) pseudo-UTP; 1-(3-Amino-propyl) pseudo-UTP; 1-(3-Cyclopropyl-prop-2-ynyl) pseudouridine TP; 1-(4-Amino-4-carboxybutyl) pseudo-UTP; 1-(4-Amino-benzyl) pseudo-UTP; 1-(4-Amino-butyl) pseudo-UTP; 1-(4-Amino-phenyl) pseudo-UTP; 1-(4-Azidobenzyl) pseudouridinc TP; 1-(4-Bromobenzyl) pseudouridine TP; 1-(4-Chlorobenzyl) pseudouridine TP; 1-(4-Fluorobenzyl) pseudouridine TP; 1-(4-lodobenzyl) pseudouridine TP; 1-(4-Methanesulfonylbenzyl) pseudouridine TP; 1-(4-Methoxybenzyl) pseudouridine TP; 1-(4-Methoxy-benzyl) pseudo-UTP; 1-(4-Methoxy-phenyl) pseudo-UTP; 1-(4-Methylbenzyl) pseudouridine TP; 1-(4-Methyl-benzyl) pseudo-UTP; 1-(4-Nitrobenzyl) pseudouridine TP; 1-(4-Nitro-benzyl) pseudo-UTP; 1-(4-Nitro-phenyl) pseudo-UTP; 1-(4-Thiomethoxybenzyl) pseudouridine TP; 1-(4-Trifluoromethoxybenzyl) pseudouridine TP; 1-(4-Trifluoromethylbenzyl) pseudouridine TP; 1-(5-Amino-pentyl) pseudo-UTP; 1-(6-Amino-hexyl) pseudo-UTP; 1,6-Dimethyl-pseudo-UTP; 1-[3-(2-{2-[2-(2-Aminoethoxy)-ethoxy]-ethoxy}-ethoxy)-propionyl]pseudouri-dine TP; 1-{3-[2-(2-Aminocthoxy)-ethoxy]-propionyl}pseudouridine TP; 1-Acetylpseudouridine TP; I-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2.2.2-Trifluoroethyl) pseudo-UTP; 1-Methyl-6-(4-morpholino)-pseudo-UTP; 1-Mcthyl-6-(4-thiomorpholino)-pseudo-UTP; 1-Methyl-6-(substituted phenyl) pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-cthylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridinc TP; 1-Pentyl-pseudo-UTP; 1-Phenyl-pseudo-UTP; 1-Pivaloylpseudouridine TP; 1-Propargylpseudouridinc TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridinc TP; 1-Thiomorpholinomethylpseudouridine TP; 1-Trifluoroacetylpseudouridine TP; 1-Trifluoromethyl-pseudo-UTP; 1-Vinylpseudouridine TP; 2.2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl) uridine TP; 3-Alkyl-pseudo-UTP; 4′-Azidouridine TP; 4′-Carbocyclic uridinc TP; 4′-Ethynyluridine TP; 5-(1-Propynyl) ara-uridine TP; 5-(2-Furanyl) uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pscudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridinc TP 1-[3-{2-(2-[2-(2-ethoxy)-ethoxy]-ethoxy)-ethoxy}]propionic acid; Pseudouridine TP 1-[3-{2-(2-[2-{2-(2-ethoxy)-ethoxy)-ethoxy}-ethoxy]-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}]propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosinc; 4-demethylwyosinc; 2,6-(diamino) purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino) purine; 2,4,5-(trimethyl)phenyl; 2′ methyl, 2′amino, 2′azido, 2′fluoro-cytidine; 2′ methyl, 2′amino, 2′azido, 2′fluoro-adenine; 2′methyl, 2′amino, 2′azido, 2′fluoro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl) isocarbostyrilyl; 3-(methyl) isocarbostyrilyl; 4-(fluoro)-6-(methyl)benzimidazole; 4-(methyl)benzimidazole; 4-(methyl) indolyl; 4,6-(dimethyl) indolyl; 5 nitroindole; 5 substituted pyrimidines; 5-(methyl) isocarbostyrilyl; 5-nitroindole; 6-(aza)pyrimidine; 6-(azo) thymine; 6-(methyl)-7-(aza) indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza) indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl) isocarbostyrilyl; 7-(propynyl) isocarbostyrilyl, propynyl-7-(aza) indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza) indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl) uridine TP; or N6-(19-Amino-pentaoxanonadecyl) adenosine TP.

In some embodiments, the adenosine-substitutable modified nucleotides comprise: 2-methylthio-N6-(cis-hydroxyisopentenyl) adenosine; 2-methylthio-N6-methyladenosine; 2-methylthio-N6-thrconyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl) adenosine; N6,2′-O-dimethyladenosine; N6,2′-O-dimethyladenosine; N6,N6.2′-O-trimethyladenosine; N6,N6-dimethyladenosine; N6-N6-hydroxynorvalylcarbamoyladenosine; N6-methyl-N6-acetyladenosine; threonylcarbamoyladenosine; 2-methyladenosine; 2-methylthio-N6-isopentenyladenosine; 7-deaza-adenosine; N1-methyl-adenosine; N6,N6 (dimethyl) adenine; N6-cis-hydroxy-isopentenyl-adenosine; alpha-thio-adenosine; 2 (amino) adenine; 2 (aminopropyl) adenine; 2 (methylthio) N6 (isopentenyl) adenine; 2-(alkyl) adenine; 2-(aminoalkyl) adenine; 2-(aminopropyl) adenine; 2-(halo) adenine; 2-(halo) adenine; 2-(propyl) adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl) adenine; 6 (methyl) adenine; 6-(alkyl) adenine; 6-(methyl) adenine; 7 (deaza) adenine; 8 (alkenyl) adenine; 8 (alkynyl) adenine; 8 (amino) adenine; 8 (thioalkyl) adenine; 8-(alkenyl) adenine; 8-(alkyl) adenine; 8-(alkynyl) adeninc; 8-(amino) adenine; 8-(halo) adeninc; 8-(hydroxyl) adenine; 8-(thioalkyl) adenine; 8-(thiol) adenine; 8-azido-adenosine; aza adenine; deaza adenine; N6 (methyl) adenine; N6-(isopentyl) adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromoadenosine TP; 8-Trifluoromethyladenosine TP; 9-Deazaadenosine TP; Wybutosine; Hydroxywybutosine; Isowyosine; Peroxywybutosine; undermodified hydroxywybutosine; 4-demethylwyosine; 2,6-(diamino) purinc; 1-(aza)-2-(thio)-3-(aza)-phenoxazin-1-yl: 1,3-(diaza)-2-(oxo)-phenthiazin-1-yl; 1.3-(diaza)-2-(oxo)-phenoxazin-1-yl; 1,3,5-(triaza)-2,6-(dioxa)-naphthalene; 2 (amino) purine; 2,4,5-(trimethyl)phenyl; 2′ methyl, 2′amino, 2′azido, 2′fluoro-cytidine; 2′ methyl, 2′amino, 2′azido, 2′fluoro-adenine; 2′methyl, 2′amino, 2′azido, 2′fluoro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl) isocarbostyrilyl; 3-(methyl) isocarbostyrilyl; 4-(fluoro)-6-(methyl)benzimidazole; 4-(methyl)benzimidazole; 4-(methyl) indolyl; 4,6-(dimethyl) indolyl; 5 nitroindole; 5 substituted pyrimidines; 5-(methyl) isocarbostyrilyl; 5-nitroindole; 6-(aza)pyrimidine; 6-(azo) thymine; 6-(methyl)-7-(aza) indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza) indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl) isocarbostyrilyl; 7-(propynyl) isocarbostyrilyl, propynyl-7-(aza) indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza) indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; 1 Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosinc TP; 5-(2-carbomethoxyvinyl) uridinc TP; or N6-(19-Amino-pentaoxanonadecyl) adenosine TP.

In some embodiments, the uridine-substitutable modified nucleotides or the thymidine-substitutable modified nucleotides comprise: pseudouridine; N1-methylpseudouridine; N1-ethylpseudouridine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Qucuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; (3-(3-amino-3-carboxypropyl) uridinc; 1-methyl-3-(3-amino-5-carboxypropyl) pseudouridine; 1-methylpseduouridinc; 1-methyl-pseudouridine; 2′-O-methyluridine; 2′-O-methylpseudouridine; 2′-O-methyluridinc; 2-thio-2′-O-methyluridine; 3-(3-amino-3-carboxypropyl) uridine; 3,2′-O-dimethyluridine; 3-Methyl-pseudo-Uridine TP; 4-thiouridine; 5-(carboxyhydroxymethyl) uridinc; 5-(carboxyhydroxymethyl) uridine methyl ester, 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester, 5-carboxymethylaminomethyl-2′-O-methyluridine; 5-carboxymethylaminomethyl-2-thiouridine; 5-carboxymethylaminomethyl-2-thiouridine; 5-caboxymethylaminomethyluridinc; 5-carboxymethylaminomethyluridine; 5-Carbamoylmethyluridine TP; 5-methoxycacoonylmethyl-2′-O-methyluridine; 5-methoxycarbonylmethyl-2-thiouridine; 5-methoxycarbonylmethyluridine; 5-methoxyuridinc; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine; N1-ethyl-pseudo-uridine; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl) uridine TP; 5-propynyl uracil;alpha-thio-uridine; 1 (aminoalkylamino-carbonylethylenyl)-2 (thio)-1 pseudouridinc; 1 (aminoalkylaminocarbonylethylenyl)-2,4-(dithio) pseudouridine; (aminoalkylaminocarbonylethylenyl)-4 1 (thio) pseudouridine; (aminoalkylaminocarbonylethylenyl)-pseudouridine; 1 (aminocazbonylethylenyl)-2 (thio)-pseudouridine; 1 (aminocarbonylethylenyl)-2,4-(dithio) pseudouridine; 1 (aminocarbonylethylenyl)-4 (thio) pseudouridinc; 1 (aminocarbonylethylenyl)-pseudouridine; 1 substituted 2 (thio)-pseudouridine; 1 substituted 2,4-(dithio) pseudouridine; 1 substituted 4 (thio) pseudouridine; 1 substituted pseudouridine; 1-(aminoalkylamino-carbonylethylenyl)-2-(thio)-pseudouridine; 1-Methyl-3-(3-amino-3-carboxypropyl) pseudouridine TP; 1-Methyl-3-(3-amino-3-carboxypropyl) pseudo-UTP; 1-Methyl-pseudo-UTP; 2 (thio) pseudouridine; 2′ deoxy uridine; 2′ fluorouridine; 2-(thio) uracil; 2.4-(dithio) psuedouracil; 2′ methyl, 2′amino, 2′azido, 2′fluoro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl) uracil; 4 (thio) pseudouridine; 4-(thio) pseudouridine; 4-(thio) uracil; 4-thiouracil; 5 (1,3-diazole-1-alkyl) uracil; 5 (2-aminopropyl) uracil; 5 (aminoalkyl) uracil; 5 (dimethylaminoalkyl) uracil; 5 (guanidiniumalkyl) uracil; 5 (methoxycarbonylmethyl)-2-(thio) uracil; 5 (methoxycarbonylmethyl) uracil; 5 (methyl) 2 (thio) uracil; 5 (methyl) 2.4 (dithio) uracil; 5 (methyl) 4 (thio) uracil; 5 (methylaminomethyl)-2 (thio) uracil; 5 (methylaminomethyl)-2,4 (dithio) uracil; 5 (methylaminomethyl)-4 (thio) uracil; 5 (propynyl) uracil; 5 (trifluoromethyl) uracil; 5-(2-aminopropyl) uracil; 5-(alkyl)-2-(thio) pseudouridine; 5-(alkyl)-2.4 (dithio) pseudouridine; 5-(alkyl)-4 (thio) pseudouridinc; 5-(alkyl) pseudouridine; 5-(alkyl) uracil; 5-(alkynyl) uracil; 5-(allylamino) uracil; 5-(cyanoalkyl) uracil; 5-(dialkylaminoalkyl) uracil; 5-(dimethylaminoalkyl) uracil; 5-(guanidiniumalkyl) uracil; 5-(halo) uracil; 5-(1,3-diazole-1-alkyl) uracil; 5-(methoxy) uracil; 5-(methoxycarbonylmethyl)-2-(thio) uracil; 5-(methoxycarbonylmethyl) uracil; 5-(methyl) 2 (thio) uracil; 5-(methyl) 2,4 (dithio) uracil; 5-(methyl) 4 (thio) uracil; 5-(methyl)-2-(thio) pseudouridine; 5-(methyl)-2,4 (dithio) pseudouridine; 5-(methyl)-4 (thio) pseudouridine; 5-(methyl) pseudouridinc; 5-(methylaminomethyl)-2 (thio) uracil; 5-(methylaminomethyl)-2.4 (dithio) uracil; 5-(methylaminomethyl)-4-(thio) uracil; 5-(propynyl) uracil; 5-(trifluoromethyl) uracil; 5-aminoallyl-uridine; 5-bromo-uridine; 5-iodouridine; 5-uracil; 6 (azo) uracil; 6-(azo) uracil; 6-aza-uridine; allyamino-uracil; aza uracil; deaza uracil; N3 (methyl) uracil; Pseudo-UTP-1-2-cthanoic acid; Pseudouridine; 4-Thio-pseudo-UTP; 1-carboxymethyl-pscudouridine; 1-methyl-1-deaza-pscudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pscudouridinc; 4-thio-pseudouridinc; 5-aza-uridinc; Dihydropseudouridine; (.+−.) 1-(2-Hydroxypropyl) pseudouridine TP; (2R)-1-(2-Hydroxypropyl) pseudouridine TP; (2S)-1-(2-Hydroxypropyl) pseudouridinc TP; (E)-5-(2-Bromo-vinyl) ara-uridine TP; (E)-5-(2-Bromo-vinyl) uridine TP; (Z)-5-(2-Bromo-vinyl) ara-uridine TP; (Z)-5-(2-Bromo-vinyl) uridine TP; 1-(2,2,2-Trifluoroethyl)-pseudo-UTP; 1-(2,2,3,3,3-Pentafluoropropyl) pseudouridine TP; 1-(2,2-Diethoxyethyl) pseudouridine TP; 1-(2.4,6-Trimethylbenzyl) pseudouridine TP; 1-(2,4,6-Trimethyl-benzyl) pseudo-UTP; 1-(2,4,6-Trimethyl-phenyl) pseudo-UTP; 1-(2-Amino-2-carboxyethyl) pseudo-UTP; 1-(2-Amino-ethyl) pseudo-UTP; 1-(2-Hydroxyethyl) pseudouridine TP; 1-(2-Methoxyethyl) pseudouridine TP; 1-(3,4-Bis-trifluoromethoxybenzyl) pseudouridine TP; 1-(3,4-Dimethoxybenzyl) pseudouridine TP; 1-(3-Amino-3-carboxypropyl) pseudo-UTP; 1-(3-Amino-propyl) pseudo-UTP; 1-(3-Cyclopropyl-prop-2-ynyl) pseudouridine TP; 1-(4-Amino-4-carboxybutyl) pseudo-UTP; 1-(4-Amino-benzyl) pseudo-UTP; 1-(4-Amino-butyl) pseudo-UTP; 1-(4-Amino-phenyl) pseudo-UTP; 1-(4-Azidobenzyl) pseudouridine TP 1-(4-Bromobenzyl) pseudouridine TP; 1-(4-Chlorobenzyl) pseudouridine TP: 1-(4-Fluorobenzyl) pseudouridine TP; 1-(4-Iodobenzyl) pseudouridine TP; 1-(4-Methanesulfonylbenzyl) pseudouridine TP; 1-(4-Methoxybenzyl) pseudouridine TP; 1-(4-Methoxy-benzyl) pseudo-UTP; Methylbenzyl) pseudouridine TP 1-(4-Methyl-benzyl) pseudo-UTP; 1-(4-Nitrobenzyl) pseudouridine TP; 1-(4-Nitro-benzyl) pseudo-UTP; 1-(4-Nitro-phenyl) pseudo-UTP; 1-(4-Methoxy-phenyl) pseudo-UTP; 1-(4-1-(4-Thiomethoxybenzyl) pseudouridine TP; 1-(4-Trifluoromethoxybenzyl) pseudouridine TP; 1-(4-Trifluoromethylbenzyl) pseudouridine TP; 1-(5-Amino-pentyl) pseudo-UTP; 1-(6-Amino-hexyl) pseudo-UTP; 1,6-Dimethyl-pseudo-UTP; 1-[3-(2-{2-[2-(2-Aminoethoxy)-ethoxy]-ethoxy}-ethoxy)-propionyl]pseudouri-dine TP; 1-{3-[2-(2-Aminoethoxy)-ethoxy]-propionyl}pseudouridine TP; 1-Acetylpseudouridine TP; I-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2.2.2-Trifluoroethyl) pseudo-UTP; 1-Methyl-6-(4-morpholino)-pseudo-UTP; 1-Methyl-6-(4-thiomorpholino)-pseudo-UTP; 1-Methyl-6-(substituted phenyl) pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2.2′-anhydro-uridine TP; 2′-bromo-deoxyuridine TP; 2′-F-5-Methyl-2′-deoxy-UTP; 2′-OMe-5-Mc-UTP; 2′-OMe-pscudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′0.2′-difluorouridine TP; 2′-Deoxy-2′-a-mercaptouridine TP; 2′-Deoxy-2′-a-thiomethoxyuridine TP; 2′-Deoxy-2′-b-aminouridinc TP; 2′-Deoxy-2′-b-azidouridine TP; 2′-Deoxy-2′-b-bromouridine TP; 2′-Deoxy-2′-b-chlorouridinc TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl) uridine TP; 3-Alkyl-pseudo-UTP; 4′-Azidouridine TP; 4′-Carbocyclic uridinc TP; 4′-Ethynyluridine TP; 5-(1-Propynyl) ara-uridine TP; 5-(2-Furanyl) uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-lodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2-(2-ethoxy)-ethoxy)-ethoxy}-ethoxy]-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}]propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino) purine; 1-(aza)-2-(thio)-3-(aza)-phenoxazin-1-yl: 1,3-(diaza)-2-(oxo)-phenthiazin-1-yl; 1.3-(diaza)-2-(oxo)-phenoxazin-1-yl; 1,3,5-(triaza)-2,6-(dioxa)-naphthalene; 2 (amino) purine; 2,4,5-(trimethyl)phenyl; 2′ methyl, 2′amino, 2′azido, 2′fluoro-cytidine; 2′ methyl, 2′amino, 2′azido, 2′fluoro-adenine; 2′methyl, 2′amino, 2′azido, 2′fluoro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl) isocarbostyrilyl; 3-(methyl) isocarbostyrilyl; 4-(fluoro)-6-(methyl)benzimidazole; 4-(methyl)benzimidazole; 4-(methyl) indolyl; 4,6-(dimethyl) indolyl; 5 nitroindole; 5 substituted pyrimidines; 5-(methyl) isocarbostyrilyl; 5-nitroindole; 6-(aza)pyrimidine; 6-(azo) thymine; 6-(methyl)-7-(aza) indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-7-(aza) indolyl; (guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl) isocarbostyrilyl; 7-(propynyl) isocarbostyrilyl, propynyl-7-(aza) indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza) indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthinc; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-cytidine TP; 2′-OH-ara-uridine TP TP; or 5-(2-carbomethoxyvinyl) uridine TP.

In some embodiments, the cytosine-substitutable modified nucleotides comprise 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; alpha-thio-cytidine; 2-(thio) cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza) cytosine; 3 (methyl) cytosinc; 3-(alkyl) cytosinc; 3-(deaza) 5 (aza) cytosine; 3-(methyl) cytidine; 4,2′-O-dimethylcytidine; 5 (halo) cytosine; 5 (methyl) cytosine; 5 (propynyl) cytosine; 5 (trifluoromethyl) cytosine; 5-(alkyl) cytosine; 5-(alkynyl) cytosine; 5-(halo) cytosine; 5-(propynyl) cytosine; 5-(trifluoromethyl) cytosine: 5-bromo-cytidine; 5-iodo-cytidine; 5-propynyl cytosine; 6-(azo) cytosine; 6-aza-cytidine; aza cytosine; deaza cytosine; N4 (acetyl) cytosine; 1-methyl-1-deaza-pscudoisocytidine; 1-methyl-pseudoisocytidine; 2-methoxy-5-methyl-cytidine: 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl) cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl) cytidine TP; 3′-Ethynylcytidine TP; 4′-Azidocytidine TP; 4′-Carbocyclic cytidine TP; 4′-Ethynylcytidine TP; 5-(1-Propynyl) ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 2′fluoro-cytidine; or 2′-OH-ara-cytidine TP.

In some embodiments, the modified nucleotides comprise: 7-methylguanosine; N2,2′-O-dimethylguanosine; N2-methylguanosine; Wyosinc; 1,2′-O-dimethylguanosine; 1-methylguanosine; 2′-O-methylguanosine; 2′-O-ribosylguanosine (phosphate); 2′-O-methylguanosine; 2′-O-ribosylguanosinc (phosphate); 7-aminomethyl-7-deazaguanosine; 7-cyano-7-deazaguanosine; Archacosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; alpha-thio-guanosine; 2 (propyl) guanine; 2-(alkyl) guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl) guanine; 6-(alkyl) guanine; 6-(methyl) guanine; 6-methyl-guanosinc; 7 (alkyl) guanine; 7 (deaza) guanine; 7 (methyl) guanine; 7-(alkyl) guanine; 7-(dcaza) guanine; 7-(methyl) guanine; 8 (alkyl) guaninc; 8 (alkynyl) guanine; 8 (halo) guanine; 8 (thioalkyl) guanine; 8-(alkenyl) guanine; 8-(alkyl) guanine; 8-(alkynyl) guaninc; 8-(amino) guanine; 8-(halo) guanine; 8-(hydroxyl) guanine; 8-(thioalkyl) guanine; 8-(thiol) guanine; aza guanine; deaza guanine; N(methyl) guanine; N-(methyl) guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosinc TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl) isocarbostyrilyl; 7-(propynyl) isocarbostyrilyl, propynyl-7-(aza) indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza) indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; or 2′-OH-ara-guanosine TP.

The mRNA may encode more than one antigen. For example, the mRNA encoding an antigen protein may encode only the antigen or may encode additional proteins. Each antigen and additional protein(s) may be under the control of different regulatory elements. Alternatively, the antigen and additional proteins may be under the control of the same regulatory element. Where at least two additional proteins are encoded, some of the antigen and additional proteins may be under the control of the same regulatory element and some may be under the control of different regulatory elements.

mRNA may be non-replicating or may be replicating, also known as self-replicating. A self-replicating mRNA molecule may be an alphavirus-derived mRNA replicon. mRNA amplification can also be achieved by the provision of a non-replicating mRNA encoding an antigen in conjunction with a separate mRNA encoding replication machinery.

Self-replicating RNA molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded antigen, or may be transcribed to provide further transcripts with the same sense as the delivered RNA which are translated to provide in situ expression of the antigen. The overall result of this sequence of transcriptions is a huge amplification in the number of the introduced replicon RNAs and so the encoded antigen becomes a major polypeptide product of the cells.

Suitable alphavirus replicons can use a replicase from a Sindbis virus, a Semliki forest virus, an eastern equine encephalitis virus, a Venezuelan equine encephalitis virus, etc. Mutant or wild-type virus sequences can be used, e.g., the attenuated TC83 mutant of VEEV has been used in replicons, (see, WO2005/113782).

In certain embodiments, the self-replicating RNA molecule described herein encodes (i) an RNA-dependent RNA polymerase which can transcribe RNA from the self-replicating RNA molecule and (ii) an antigen. The polymerase can be an alphavirus replicase, e.g., comprising one or more of alphavirus proteins nsP1, nsP2, nsP3 and nsP4.

Whereas natural alphavirus genomes encode structural virion proteins in addition to the non-structural replicase polyprotein, in certain embodiments, the self-replicating RNA molecules do not encode alphavirus structural proteins. Thus, the self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the present disclosure and their place is taken by gene(s) encoding the antigen/immunogen of interest, such that the subgenomic transcript encodes the immunogen rather than the structural alphavirus virion proteins.

Thus, a self-replicating RNA molecule useful with the invention may have two open reading frames. The first (5′) open reading frame encodes a replicase; the second (3′) open reading frame encodes an antigen. In some embodiments the RNA may have additional (e.g., downstream) open reading frames, e.g., to encode further antigens or to encode accessory polypeptides.

In certain embodiments, the self-replicating RNA molecule disclosed herein has a 5′ cap (e.g., a 7-methylguanosine). This cap can enhance in vivo translation of the RNA. In some embodiments the 5′ sequence of the self-replicating RNA molecule must be selected to ensure compatibility with the encoded replicase.

A self-replicating RNA molecule may have a 3′ poly-A tail. It may also include a poly-A polymerase recognition sequence (e.g., AAUAAA) near its 3′ end.

Self-replicating RNA molecules can have various lengths, but are typically 5000 to 25000 nucleotides long, such as 8000 to 15000 nucleotides long, for example 9000 to 12000 nucleotides long. Self-replicating RNA molecules will typically be single-stranded. Single-stranded RNAs can generally initiate an adjuvant effect by binding to TLR7, TLR8, RNA helicases and/or PKR. RNA delivered in double-stranded form (dsRNA) can bind to TLR3, and this receptor can also be triggered by dsRNA which is formed either during replication of a single-stranded RNA or within the secondary structure of a single-stranded RNA.

In another embodiment, a self-replicating RNA may comprise two separate RNA molecules, each comprising a nucleotide sequence derived from an alphavirus: one RNA molecule comprises an RNA construct for expressing alphavirus replicase, and one RNA molecule comprises an RNA replicon that can be replicated by the replicase in trans. In some embodiments, the RNA construct for expressing alphavirus replicase comprises a 5′-cap. WO2017/162265.

The self-replicating RNA can conveniently be prepared by in vitro transcription (IVT). IVT can use a (cDNA) template created and propagated in plasmid form in bacteria, or created synthetically (for example by gene synthesis and/or polymerase chain-reaction (PCR) engineering methods). For instance, a DNA-dependent RNA polymerase (such as the bacteriophage T7, T3 or SP6 RNA polymerases) can be used to transcribe the self-replicating RNA from a DNA template. Appropriate capping and poly-A addition reactions can be used as required (although the replicon's poly-A is usually encoded within the DNA template). These RNA polymerases can have stringent requirements for the transcribed 5′ nucleotide(s) and in some embodiments these requirements must be matched with the requirements of the encoded replicase, to ensure that the IVT-transcribed RNA can function efficiently as a substrate for its self-encoded replicase.

A self-replicating RNA can include (in addition to any 5′ cap structure) one or more nucleotides having a modified nucleobase. An RNA used with the invention ideally includes only phosphodiester linkages between nucleosides, but in some embodiments it can contain phosphoramidate, and/or methylphosphonate linkages.

The self-replicating RNA molecule may encode a single heterologous polypeptide antigen (i.e. the antigen) or, optionally, two or more heterologous polypeptide antigens linked together in a way that each of the sequences retains its identity (e.g., linked in series) when expressed as an amino acid sequence. The heterologous polypeptides generated from the self-replicating RNA may then be produced as a fusion polypeptide or engineered in such a manner to result in separate polypeptide or peptide sequences.

The self-replicating RNA molecules described herein may be engineered to express multiple nucleotide sequences, from two or more open reading frames, thereby allowing co-expression of proteins, such as one, two or more antigens (e.g. one, two or more protein(s) together with cytokines or other immunomodulators, which can enhance the generation of an immune response). Such a self-replicating RNA molecule might be particularly useful, for example, in the production of various gene products (e.g., proteins) at the same time, for example, as a bivalent or multivalent vaccine.

If desired, the self-replicating RNA molecules can be screened or analyzed to confirm their therapeutic and prophylactic properties using various in vitro or in vivo testing methods that are known to those of skill in the art. For example, vaccines comprising self-replicating RNA molecule can be tested for their effect on induction of proliferation or effector function of the particular lymphocyte type of interest, e.g., B cells, T cells, T cell lines, and T cell clones. For example, spleen cells from immunized mice can be isolated and the capacity of cytotoxic T lymphocytes to lyse autologous target cells that contain a self-replicating RNA molecule that encodes an antigen can be tested. In addition, T helper cell differentiation can be analyzed by measuring proliferation or production of TH1 (IL-2 and IFN-γ) and/or TH2 (IL-4 and IL-5) cytokines by ELISA or directly in CD4+ T cells by cytoplasmic cytokine staining and flow cytometry.

Self-replicating RNA molecules that encode an antigen can also be tested for ability to induce humoral immune responses, as evidenced, for example, by induction of B cell production of antibodies specific for the antigen of interest. These assays can be conducted using, for example, peripheral B lymphocytes from immunized individuals. Such assay methods are known to those of skill in the art. Other assays that can be used to characterize the self-replicating RNA molecules can involve detecting expression of the encoded antigen by the target cells. For example, FACS can be used to detect antigen expression on the cell surface or intracellularly. Another advantage of FACS selection is that one can sort for different levels of expression; sometimes-lower expression may be desired. Other suitable methods for identifying cells which express a particular antigen involve panning using monoclonal antibodies on a plate or capture using magnetic beads coated with monoclonal antibodies.

A single dose of SAM may be 1-30 ug/mL.

A non-replicating mRNA will typically contain 10000 bases or fewer, especially 8000 bases or fewer, in particular 5000 bases or fewer. A replicating mRNA will typically contain 25000 bases or fewer, especially 20000 bases or fewer, in particular 15000 bases or fewer. A replicating mRNA may contain 5000 to 25000 nucleotides, such as 8000 to 15000 nucleotides, for example 9000 to 12000 nucleotides.

A single dose of mRNA may be 1 to 1000 ug, especially 1 to 500 ug, in particular 10 to 250 ug. A single dose of mRNA may be 1 to 75 ug. 1 to 75 ug, 25 to 250 ug, or 250 to 1000 ug. Specifically, a replicating mRNA dose may be 1 to 75 ug, such as 1 to 75 ug. A non-replicating mRNA dose may, for example, be 1 to 500 ug, such as 1 to 250 ug.

In one embodiment the mRNA is non-replicating mRNA. In a second embodiment the mRNA is replicating mRNA.

A preferred self-replicating RNA molecule thus encodes (i) a RNA-dependent RNA polymerase which can transcribe RNA from the self-replicating RNA molecule and (ii) an immunogen. The polymerase can be an alphavirus replicase e.g. comprising one or more of alphavirus proteins nsP1, nsP2, nsP3 and nsP4.

A self-replicating RNA can include one or more modified pyrimidine nucleobases, such as pseudouridine and/or 5-methylcytosine residues. In some embodiments, however, the RNA includes no modified nucleobases, and may include no modified nucleotides i.e. all of the nucleotides in the RNA are standard A, C, G and U ribonucleotides (except for any 5′ cap structure, which may include a 7′-methylguanosine). In other embodiments, the RNA may include a 5′ cap comprising a 7′-methylguanosine, and the first 1, 2 or 3 5′ ribonucleotides may be methylated at the 2′ position of the ribose.

### Amount of RNA in a Vaccine Composition

The RNA present in the vaccine composition is present as provided in this application. A single dose of mRNA may be about 0.1 to 60 μg or more. A replicating mRNA dose (such as a SAM dose) will usually be lower than a non-replicating mRNA dose encoding the same immunogen. A non-replicating mRNA dose may be, for example, 0.1 to 60 ug, or more. Where the composition (or container such as a vial holding the composition) contains multiple doses, the composition can contain multiples of the above amounts (and may also include overfill). Thus, in accordance with the present invention, a container holding the composition can contain a single dose or multiple doses.

Generally speaking, SAM-LNPs are formulated based on RNA concentration/mass. Ideally, the concentration of RNA (such as mRNA or SAM) pre-lyophilization and post-lyophilization should be about the same. Thus, for example, with a SAM-LNP concentration of 1 μg/mL, 700 μL of SAM-LNP can be filled into a lyophilization vial and the lyophilization vial should theoretically contain 0.7 ug of RNA. During lyophilization, the volume of the SAM-LNP product may expand because of the lyophilization process. Post lyophilization, the lyophilized product such as lyophilized powder and/or cake is reconstituted in 646 μL of water for injection (WFI). Upon reconstitution, the concentration of SAM-LNP is expected to be about the same as the volume pre-lyophilization (and mostly is around that upon testing using a RiboGreen assay). When 0.5 mL is dosed (500 L-human dose), it is expected that the dose will be 0.5 ug.

Unless otherwise indicated, the amount or concentration of SAM is the amount of the same that is encapsulated in an LNP and does not include any amount that is outside of the LNP, which is expected to be only a very small amount (such as less than 5%, less than 4%, less than 3%, less than 2%, less than 1% or less than 0.5%, if any at all). In accordance with this embodiment, a vaccine composition is an effective amount of SAM-LNP of about 0.1 to 60 μg of SAM or more. More specifically, SAM-LNP can be present in an effective amount such as 0.5 to 30 μg of SAM, 0.5 to 20 μg of SAM or 1 to 10 μg of SAM, etc.

### The Immunogen/Antigen

RNA molecules encapsulated within a LNP encode a polypeptide immunogen. After administration of the RNA the immunogen is translated in vivo and can elicit an immune response in the recipient. The immunogen may elicit an immune response against a bacterium, a virus, a fungus or a parasite (or, in some embodiments, against an allergen; and in other embodiments, against a tumor antigen). The immune response may comprise an antibody response (usually including IgG) and/or a cell-mediated immune response. The polypeptide immunogen will typically elicit an immune response which recognises the corresponding bacterial, viral, fungal or parasite (or allergen or tumour) polypeptide, but in some embodiments the polypeptide may act as a mimotope to elicit an immune response which recognises a bacterial, viral, fungal or parasite saccharide. The immunogen will typically be a surface polypeptide e.g. an adhesin, a hemagglutinin, an envelope glycoprotein, a spike glycoprotein, etc.

RNA molecules can encode a single polypeptide immunogen or multiple polypeptides. Multiple immunogens can be presented as a single polypeptide immunogen (fusion polypeptide) or as separate polypeptides. If immunogens are expressed as separate polypeptides then one or more of these may be provided with an upstream IRES or an additional viral promoter element. Alternatively, multiple immunogens may be expressed from a polyprotein that encodes individual immunogens fused to a short autocatalytic protease (e.g., foot-and-mouth disease virus 2A protein).

Unlike Johanning et al. (1995) Nucleic Acids Res 23:1495-1501 and El Ouahabi et al. (1996) FEBS Letts 380:108-12, the RNA encodes an immunogen. For the avoidance of doubt, the invention does not encompass RNA which encodes a firefly luciferase or which encodes a fusion protein of E. coli β-galactosidase or which encodes a green fluorescent protein (GFP). Also, the RNA is not total mouse thymus RNA.

In some embodiments the immunogen elicits an immune response against one of these bacteria:

Neisseria meningitidis: useful immunogens include, but are not limited to, membrane proteins such as adhesins, autotransporters, toxins, iron acquisition proteins, and factor H binding protein. A combination of three useful polypeptides is disclosed in Giuliani et al. (2006) Proc Natl Acad Sci USA 103 (29): 10834-9.

Streptococcus pneumoniae: useful polypeptide immunogens are disclosed in WO2009/016515. These include, but are not limited to, the RrgB pilus subunit, the beta-N-acetyl-hexosaminidase precursor (spr0057), spr0096, General stress protein GSP-781 (spr2021, SP2216), serine/threonine kinase StkP (SP1732), and pneumococcal surface adhesin PsaA.

Streptococcus pyogenes: useful immunogens include, but are not limited to, the polypeptides disclosed in WO02/34771 and WO2005/032582.

Moraxella catarrhalis.

Bordetella pertussis: Useful pertussis immunogens include, but are not limited to, pertussis toxin or toxoid (PT), filamentous haemagglutinin (FHA), pertactin, and agglutinogens 2 and 3.

Staphylococcus aureus: Useful immunogens include, but are not limited to, the polypeptides disclosed in WO2010/119343 such as a hemolysin, esxA, esxB, ferrichrome-binding protein (sta006) and/or the sta011 lipoprotein.

Clostridium tetani: the typical immunogen is tetanus toxoid.

Cornynebacterium diphtheriae: the typical immunogen is diphtheria toxoid.

Haemophilus influenzae: Useful immunogens include, but are not limited to, the polypeptides disclosed in WO2006/110413 and WO2005/111066.

Pseudomonas aeruginosa

Streptococcus agalactiae: useful immunogens include, but are not limited to, the polypeptides disclosed in WO2006/089264.

Chlamydia trachomatis: Useful immunogens include, but are not limited to, PepA, LcrE, ArtJ, DnaK, CT398, OmpH-like, L7/L12, OmcA, AtoS, CT547, Eno, HtrA and MurG (e.g. as disclosed in WO2005/002619 LcrE (see, WO2006/138004) and HtrA (see, WO2009/109860) are two preferred immunogens.

Chlamydia pneumoniae: Useful immunogens include, but are not limited to, the polypeptides disclosed in WO02/02606.

Helicobacter pylori: Useful immunogens include, but are not limited to, CagA, VacA, NAP, and/or urease WO03/018054.

Escherichia coli: Useful immunogens include, but are not limited to, immunogens derived from enterotoxigenic E. coli (ETEC), enteroaggregative E. coli (EAggEC), diffusely adhering E. coli (DAEC), enteropathogenic E. coli (EPEC), extraintestinal pathogenic E. coli (ExPEC) and/or enterohemorrhagic E. coli (EHEC). ExPEC strains include uropathogenic E. coli (UPEC) and meningitis/sepsis-associated E. coli (MNEC). Useful UPEC polypeptide immunogens are disclosed in WO2006/091517 and WO2008/020330. Useful MNEC immunogens are disclosed in WO2006/089264. A useful immunogen for several E. coli types is AcfD (see, WO2009/104092).
Bacillus anthracis
Yersinia pestis: Useful immunogens include, but are not limited to, those disclosed in
WO2009/031043 and WO2007/049155.
Staphylococcus epidermis
Clostridium perfringens or Clostridium botulinums
Legionella pneumophila
Coxiella burnetii
Brucella, such as B.abortus, B.canis, B.melitensis, B.neotomae, B.ovis, B.suis, B.pinnipediae.
Francisella, such as F.novicida, F.philomiragia, F.tularensis.
Neisseria gonorrhoeae
Treponema pallidum
Haemophilus ducreyi
Enterococcus faecalis or Enterococcus faecium
Staphylococcus saprophyticus
Yersinia enterocolitica
Mycobacterium tuberculosis
Rickettsia
Listeria monocytogenes
Vibrio cholerae
Salmonella typhi
Borrelia burgdorferi
Porphyromonas gingivalis
Klebsiella

In some embodiments the immunogen elicits an immune response against one of these viruses:

Orthomyxovirus: Useful immunogens can be from an influenza A, B or C virus, such as the hemagglutinin, neuraminidase or matrix M2 proteins. Where the immunogen is an influenza A virus hemagglutinin it may be from any subtype e.g. H1, H2, H3, H4, H5, H6, H7, H8, H9, H10, H11, H12, H13, H14, H15 or H16.

Paramyxoviridae viruses: Viral immunogens include, but are not limited to, those derived from Pneumoviruses (e.g. respiratory syncytial virus, RSV), Rubulaviruses (e.g. mumps virus), Paramyxoviruses (e.g. parainfluenza virus), Metapneumoviruses and Morbilliviruses (e.g. measles).

Poxviridae: Viral immunogens include, but are not limited to, those derived from Orthopoxvirus such as Variola vera, including but not limited to, Variola major and Variola minor and Monkeypox vius.

Picornavirus: Viral immunogens include, but are not limited to, those derived from Picornaviruses, such as Enteroviruses, Rhinoviruses, Heparnavirus, Cardioviruses and Aphthoviruses. In one embodiment, the enterovirus is a poliovirus e.g. a type 1, type 2 and/or type 3 poliovirus. In another embodiment, the enterovirus is an EV71 enterovirus. In another embodiment, the enterovirus is a coxsackie A or B virus.

Bunyavirus: Viral immunogens include, but are not limited to, those derived from an Orthobunyavirus, such as California encephalitis virus, a Phlebovirus, such as Rift Valley Fever virus, or a Nairovirus, such as Crimean-Congo hemorrhagic fever virus.

Heparnavirus: Viral immunogens include, but are not limited to, those derived from a Heparnavirus, such as hepatitis A virus (HAV).

Filovirus: Viral immunogens include, but are not limited to, those derived from a filovirus, such as an Ebola virus (including a Zaire, Ivory Coast, Reston or Sudan ebolavirus) or a Marburg virus.

Togavirus: Viral immunogens include, but are not limited to, those derived from a Togavirus, such as a Rubivirus, an Alphavirus, or an Arterivirus. This includes rubella virus.

Flavivirus: Viral immunogens include, but are not limited to, those derived from a Flavivirus, such as Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus, Powassan encephalitis virus.

Pestivirus: Viral immunogens include, but are not limited to, those derived from a Pestivirus, such as Bovine viral diarrhea (BVDV), Classical swine fever (CSFV) or Border disease (BDV).

Hepadnavirus: Viral immunogens include, but are not limited to, those derived from a Hepadnavirus, such as Hepatitis B virus. A composition can include hepatitis B virus surface antigen (HBsAg).

Other hepatitis viruses: A composition can include an immunogen from a hepatitis C virus, delta hepatitis virus, hepatitis E virus, or hepatitis G virus.

Rhabdovirus: Viral immunogens include, but are not limited to, those derived from a Rhabdovirus, such as a Lyssavirus (e.g. a Rabies virus) and Vesiculovirus (VSV).

Caliciviridae: Viral immunogens include, but are not limited to, those derived from Calciviridae, such as Norwalk virus (Norovirus), and Norwalk-like Viruses, such as Hawaii Virus and Snow Mountain Virus.

Coronavirus: Viral immunogens include, but are not limited to, those derived from a SARS coronavirus, avian infectious bronchitis (IBV), Mouse hepatitis virus (MHV), and Porcine transmissible gastroenteritis virus (TGEV). The coronavirus immunogen may be a spike polypeptide.

Viral immunogens include, but are not limited to, those derived from a SARS coronavirus, COVID-19 (including various strains thereof), avian infectious bronchitis (IBV), Mouse hepatitis virus (MHV), and Porcine transmissible gastroenteritis virus (TGEV). The coronavirus immunogen may be a spike polypeptide. See, e.g., Wrapp et al. (2020) “Cryo-EM structure of the 2019-nCOV spike in the prefusion conformation.” Science, 367:1260-1263. Coronavirus spike polypeptides or proteins, including fragments and modifications thereof, are known in the art and are included as immunogens. See, also, WO 2021/245611 entitled Modified Betacoronaviurs Spike Proteins.

Retrovirus: Viral immunogens include, but are not limited to, those derived from an Oncovirus, a Lentivirus (e.g. HIV-1 or HIV-2) or a Spumavirus.

Reovirus: Viral immunogens include, but are not limited to, those derived from an Orthoreovirus, a Rotavirus, an Orbivirus, or a Coltivirus.

Parvovirus: Viral immunogens include, but are not limited to, those derived from Parvovirus B19.

Herpesvirus: Viral immunogens include, but are not limited to, those derived from a human herpesvirus, such as, by way of example only, Herpes Simplex Viruses (HSV) (e.g. HSV types 1 and 2), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8).

Papovaviruses: Viral immunogens include, but are not limited to, those derived from Papillomaviruses and Polyomaviruses. The (human) papillomavirus may be of serotype 1, 2, 4, 5, 6, 8, 11, 13, 16, 18, 31, 33, 35, 39, 41, 42, 47, 51, 57, 58, 63 or 65 e.g. from one or more of serotypes 6, 11, 16 and/or 18.

Adenovirus: Viral immunogens include those derived from adenovirus serotype 36 (Ad-36).

In some embodiments, the immunogen elicits an immune response against a virus which infects fish, such as: infectious salmon anemia virus (ISAV), salmon pancreatic disease virus (SPDV), infectious pancreatic necrosis virus (IPNV), channel catfish virus (CCV), fish lymphocystis disease virus (FLDV), infectious hematopoietic necrosis virus (IHNV), koi herpesvirus, salmon picorna-like virus (also known as picorna-like virus of atlantic salmon), landlocked salmon virus (LSV), atlantic salmon rotavirus (ASR), trout strawberry disease virus (TSD), coho salmon tumor virus (CSTV), or viral hemorrhagic septicemia virus (VHSV).

Fungal immunogens may be derived from Dermatophytres, including: Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var, album, var, discoides, var, ochraceum, Trichophyton violaceum, and/or Trichophyton faviforme; or from Aspergillus fumigatus, Aspergillus flavus, Aspergillus niger, Aspergillus nidulans, Aspergillus terreus, Aspergillus sydowii, Aspergillus flavatus, Aspergillus glaucus, Blastosehizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi; the less common are Brachiola spp, Microsporidium spp., Nosema spp., Pleistophora spp., Trachipleistophora spp., Vittaforma spp Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

In some embodiments the immunogen elicits an immune response against a parasite from the Plasmodium genus, such as P.falciparum, P.vivax, P.malariae or P.ovale. Thus the invention may be used for immunising against malaria. In some embodiments the immunogen elicits an immune response against a parasite from the Caligidae family, particularly those from the Lepeophtheirus and Caligus genera e.g. sea lice such as Lepeophtheirus salmonis or Caligus rogercresseyi.

In some embodiments the immunogen elicits an immune response against: pollen allergens (trec-, herb, weed-, and grass pollen allergens); insect or arachnid allergens (inhalant, saliva and venom allergens, e.g. mite allergens, cockroach and midges allergens, hymenopthera venom allergens); animal hair and dandruff allergens (from e.g. dog, cat, horse, rat, mouse, etc.); and food allergens (e.g. a gliadin). Important pollen allergens from trees, grasses and herbs are such originating from the taxonomic orders of Fagales, Oleales, Pinales and platanaceae including, but not limited to, birch (Betula), alder (Alnus), hazel (Corylus), hornbeam (Carpinus) and olive (Olca), cedar (Cryptomeria and Juniperus), plane tree (Platanus), the order of Poales including grasses of the genera Lolium, Phleum, Poa, Cynodon, Dactylis, Holcus, Phalaris, Secale, and Sorghum, the orders of Asterales and Urticales including herbs of the genera Ambrosia, Artemisia, and Parietaria. Other important inhalation allergens are those from house dust mites of the genus Dermatophagoides and Euroglyphus, storage mite e.g. Lepidoglyphys, Glycyphagus and Tyrophagus, those from cockroaches, midges and fleas e.g. Blatella, Periplaneta, Chironomus and Ctenocepphalides, and those from mammals such as cat, dog and horse, venom allergens including such originating from stinging or biting insects such as those from the taxonomic order of Hymenoptera including bees (Apidae), wasps (Vespidea), and ants (Formicoidae).

In some embodiments the immunogen is a tumor antigen selected from: (a) cancer-testis antigens such as NY-ESO-1, SSX2, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors; (b) mutated antigens, for example, p53 (associated with various solid tumors, e.g., colorectal, lung, head and neck cancer), p21/Ras (associated with, e.g., melanoma, pancreatic cancer and colorectal cancer), CDK4 (associated with, e.g., melanoma), MUM1 (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT; (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g., renal cancer), aldolase A (associated with, e.g., lung cancer), PRAME (associated with, e.g., melanoma), HER-2/ncu (associated with, e.g., breast, colon, lung and ovarian cancer), mammaglobin, alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer); (d) shared antigens, for example, melanoma-melanocyte differentiation antigens such as MART-1/Melan A, gp100, MCIR, melanocyte-stimulating hormone receptor, tyrosinase, tyrosinase related protein-1/TRP1 and tyrosinase related protein-2/TRP2 (associated with, e.g., melanoma); (c) prostate associated antigens such as PAP, PSA, PSMA, PSH-P1, PSM-P1, PSM-P2, associated with e.g., prostate cancer; (f) immunoglobulin idiotypes (associated with myeloma and B cell lymphomas, for example). In certain embodiments, tumor immunogens include, but are not limited to, p15, Hom/Mel-40, H-Ras, E2A-PRL, H4-RET, IGH-IGK, MYL-RAR. Epstein Barr virus antigens, EBNA, human papillomavirus (HPV) antigens, including E6 and E7, hepatitis B and C virus antigens, human T-cell lymphotropic virus antigens, TSP-180, p185erbB2, p180erbB-3, c-met, mn-23H1, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29\BCAA), CA 195, CA 242, CA-50, CAM43, CD68\KP1, CO-029, FGF-5, Ga733 (EpCAM), HTgp-175, M344, MA-50, MG7-Ag, MOV18, NB/70K, NY-CO-1, RCAS1, SDCCAG16, TA-90 (Mac-2 binding protein/cyclophilin C-associated protein), TAAL6, TAG72, TLP, TPS, and the like.

Lipid Nanoparticles (mRNA Carriers)

A range of carrier systems have been described which encapsulate mRNA in order to facilitate mRNA delivery and consequent expression of encoded antigens as compared to mRNA which is not encapsulated. The present invention may utilise any suitable carrier system. Particular mRNA carrier systems of note are further described below.

### LNPs

Lipid nanoparticles (LNPs) are non-virion liposome particles in which mRNA can be encapsulated. LNP delivery systems and methods for their preparation are known in the art (see Liposomes: Methods and Protocols, Volume 1: Pharmaceutical Nanocarriers: Methods and Protocols. (ed. Weissig). Humana Press, 2009. ISBN 160327359X and Liposome Technology, volumes I, II & III. (ed. Gregoriadis). Informa Healthcare, 2006) and involves mixing (i) an ethanolic solution of the lipids (ii) an aqueous solution of the nucleic acid and (iii) buffer, followed by mixing, equilibration, dilution and purification. Preferred liposomes of the invention are obtainable by this mixing process. RNA is preferably encapsulated within the liposomes, and the liposome forms an outer layer around an aqueous RNA-containing core. This encapsulation has been found to protect RNA from RNase digestion. The particles can include some external mRNA (e.g. on the surface of the particles), but desirably at least half of the RNA (and suitably at least 85%, especially at least 95%, 96%, 97%, 98% or 99%, such as all of it) is encapsulated.

Various amphiphilic lipids can form bilayers in an aqueous environment to encapsulate a RNA-containing aqueous core as a liposome. These lipids can have an anionic, cationic or zwitterionic hydrophilic head group. Some phospholipids are anionic whereas other are zwitterionic and others are cationic. Suitable classes of phospholipid include, but are not limited to, phosphatidylethanolamines, phosphatidylcholines, phosphatidylserines, and phosphatidylglycerols, and some useful phospholipids are listed in Table 1 below:

TABLE 1
Useful Phospholipids

DDPC 1,2-Didecanoyl-sn-Glycero-3-phosphatidylcholine
DEPA 1,2-Dierucoyl-sn-Glycero-3-Phosphate
DEPC 1,2-Erucoyl-sn-Glycero-3-phosphatidylcholine
DEPE 1,2-Dierucoyl-sn-Glycero-3-phosphatidylethanolamine
DEPG 1,2-Dierucoyl-sn-Glycero-3[Phosphatidyl-rac-(1-glycerol . . .)
DLOPC 1,2-Linoleoyl-sn-Glycero-3-phosphatidylcholine
DLPA 1,2-Dilauroyl-sn-Glycero-3-Phosphate
DLPC 1,2-Dilauroyl-sn-Glycero-3-phosphatidylcholine
DLPE 1,2-Dilauroyl-sn-Glycero-3-phosphatidylethanolamine
DLPG 1,2-Dilauroyl-sn-Glycero-3[Phosphatidyl-rac-(1-glycerol . . .)
DLPS 1,2-Dilauroyl-sn-Glycero-3-phosphatidylserine
DMG 1,2-Dimyristoyl-sn-glycero-3-phosphoethanolamine
DMPA1,2-Dimyristoyl-sn-Glycero-3-Phosphate
DMPC 1,2-Dimyristoyl-sn-Glycero-3-phosphatidylcholine
DMPE 1,2-Dimyristoyl-sn-Glycero-3-phosphatidylethanolamine
DMPG1,2-Myristoyl-sn-Glycero-3[Phosphatidyl-rac-(1-glycerol . . .)
DMPS 1,2-Dimyristoyl-sn-Glycero-3-phosphatidylserine
DOPA 1,2-Dioleoyl-sn-Glycero-3-Phosphate
DOPC 1,2-Dioleoyl-sn-Glycero-3-phosphatidylcholine
DOPE 1,2-Dioleoyl-sn-Glycero-3-phosphatidylethanolamine
DOPG 1,2-Dioleoyl-sn-Glycero-3[Phosphatidyl-rac-(1-glycerol . . .)
DOPS 1,2-Dioleoyl-sn-Glycero-3-phosphatidylserine
DPPA 1,2-Dipalmitoyl-sn-Glycero-3-Phosphate
DPPC 1,2-Dipalmitoyl-sn-Glycero-3-phosphatidylcholine
DPPE 1,2-Dipalmitoyl-sn-Glycero-3-phosphatidylethanolamine
DPPG 1,2-Dipalmitoyl-sn-Glycero-3[Phosphatidyl-rac-(1-glycerol . . .)
DPPS 1,2-Dipalmitoyl-sn-Glycero-3-phosphatidylserine
DPyPE1,2-diphytanoyl-sn-glycero-3-phosphoethanolamine
DSPA 1,2-Distearoyl-sn-Glycero-3-Phosphate
DSPC 1,2-Distearoyl-sn-Glycero-3-phosphatidylcholine
DSPE 1,2-Diostearpyl-sn-Glycero-3-phosphatidylethanolamine
DSPG 1,2-Distearoyl-sn-Glycero-3[Phosphatidyl-rac-(1-glycerol . . .)
DSPS 1,2-Distearoyl-sn-Glycero-3-phosphatidylserine
EPC Egg-PC
HEPC Hydrogenated Egg PC
HSPC High purity Hydrogenated Soy PC
HSPC Hydrogenated Soy PC
LYSOPC MYRISTIC 1-Myristoyl-sn-Glycero-3-phosphatidylcholine
LYSOPC PALMITIC 1-Palmitoyl-sn-Glycero-3-phosphatidylcholine
LYSOPC STEARIC 1-Stearoyl-sn-Glycero-3-phosphatidylcholine
Milk Sphingomyelin MPPC 1-Myristoy1,2-palmitoyl-sn-Glycero 3-phosphatidylcholine
MSPC 1-Myristoy1,2-stearoyl-sn-Glycero-3-phosphatidylcholine
PMPC 1-Palmitoyl,2-myristoyl-sn-Glycero-3-phosphatidylcholine
POPC 1-Palmitoyl,2-oleoyl-sn-Glycero-3-phosphatidylcholine
POPE 1-Palmitoyl-2-oleoyl-sn-Glycero-3-phosphatidylethanolamine
POPG 1,2-Dioleoyl-sn-Glycero-3[Phosphatidyl-rac-(1-glycerol) . . .]
PSPC 1-Palmitoyl,2-stearoyl-sn-Glycero-3-phosphatidylcholine
SMPC 1-Stearoyl,2-myristoyl-sn-Glycero-3-phosphatidylcholine
SOPC 1-Stearoyl,2-oleoyl-sn-Glycero-3-phosphatidylcholine
SPPC 1-Stearoyl,2-palmitoyl-sn-Glycero-3-phosphatidylcholine

Useful cationic lipids include, but are not limited to, dioleoyl trimethylammonium propane (DOTAP), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA). Zwitterionic lipids include, but are not limited to, acyl zwitterionic lipids and ether zwitterionic lipids. Examples of useful zwitterionic lipids are DPPC, DOPC and dodecylphosphocholine. The lipids can be saturated or unsaturated. The use of at least one unsaturated lipid for preparing liposomes is preferred. If an unsaturated lipid has two tails, both tails can be unsaturated, or it can have one saturated tail and one unsaturated tail.

LNP formulated mRNA may be prepared comprising mRNA, cationic lipid, and other helper lipids. Liposomal particles can, for example, be formed of a mixture of zwitterionic, cationic and anionic lipids which can be saturated or unsaturated, for example; DSPC (zwitterionic, saturated), DlinDMA (cationic, unsaturated), and/or DMG (anionic, saturated). Preferred LNPs for use with the invention include an amphiphilic lipid which can form liposomes, optionally in combination with at least one cationic lipid (such as DOTAP, DSDMA, DODMA, DLinDMA, DLenDMA, etc.). A mixture of DSPC, DlinDMA, PEG-DMG and cholesterol is particularly effective. Other useful LNPs are described in WO2011/076807; WO 2012/006372, WO2012/006376; WO2012/030901; WO2012/031046; WO2012/031043; WO2012/006378; WO2013/033563; WO2013/006825; WO2014/136086; WO2015/095340; WO2015/095346; and WO2016/037053. In some embodiments, the LNPs are RV01 liposomes, see Liposome Technology, volumes I, II & III. (ed. Gregoriadis). Informa Healthcare, 2006 and Geall et al. (2012) PNAS USA. Sep. 4; 109 (36): 14604-9.

Liposomes can be formed from a single lipid or from a mixture of lipids. A mixture may comprise (i) a mixture of anionic lipids (ii) a mixture of cationic lipids (iii) a mixture of zwitterionic lipids (iv) a mixture of anionic lipids and cationic lipids (v) a mixture of anionic lipids and zwitterionic lipids (vi) a mixture of zwitterionic lipids and cationic lipids or (vii) a mixture of anionic lipids, cationic lipids and zwitterionic lipids. Similarly, a mixture may comprise both saturated and unsaturated lipids. For example, a mixture may comprise DSPC (zwitterionic, saturated), DlinDMA (cationic, unsaturated), and/or DMG (anionic, saturated). Where a mixture of lipids is used, not all of the component lipids in the mixture need to be amphiphilic e.g. one or more amphiphilic lipids can be mixed with cholesterol.

LNP can, for example, be formed of a mixture of (i) a PEG-modified lipid (ii) a non-cationic lipid (iii) a sterol (iv) an ionisable cationic lipid. Alternatively, LNP can for example be formed of a mixture of (i) a PEG-modified lipid (ii) a non-cationic lipid (iii) a sterol (iv) a non-ionisable cationic lipid.

In certain embodiments, the LNP may comprise SAM, a cationic lipid, a zwitterionic lipid such as phosphatidylcholine, a sterol such as cholesterol, and optionally a PEGylated lipid. In certain embodiments, the LNP may comprise SAM, an ionisable cationic lipid, a zwitterionic lipid such as phosphatidylcholine, a sterol such as cholesterol, and optionally a PEGylated lipid. In some embodiments, the LNP may comprise DSPC, DlinDMA, PEG-DMG and cholesterol. In still further embodiments, the LNP may comprise DSPC, RV39, PEG and cholesterol.

The PEG-modified lipid may comprise a PEG molecule with a molecular weight of 10000 Da or less, especially 5000 Da or less, in particular 3000 Da, such as 2000 Da or less. Examples of PEG-modified lipids include PEG-distearoyl glycerol, PEG-dipalmitoyl glycerol and PEG-dimyristoyl glycerol. The PEG-modified lipid is typically present at around 0.5 to 15 molar %.

The non-cationic lipid may be a neutral lipid, such as 1,2-distearoyl-sn-glycero-3-phosphocholine (DSPC), 1,2-dipalmitoyl-sn-glycero-3-phosphocholine (DPPC), 1-palmitoyl-2-oleoyl-sn-glycero-3-phosphocholine (POPC), 1,2-dioleoyl-sn-glycero-3-phosphoethanolamine (DOPE) and sphingomyelin (SM). The non-cationic lipid is typically present at around 5 to 25 molar %.

The sterol may be cholesterol. The sterol is typically present at around 25 to 55 molar %.

A range of suitable ionizable cationic lipids are known in the art, which are typically present at around 20 to 60 molar %.

The ratio of RNA to lipid can be varied (see for example WO2013/006825). “N: P ratio” refers to the molar ratio of protonatable nitrogen atoms in the cationic lipids (typically solely in the lipid's headgroup) to phosphates in the RNA. The ratio of nucleotide (N) to phospholipid (P) can be in the range of, e.g., 1N: 1P to 20N: 1P, 1N: 1P to 10N: 1P, 2N: 1P to 8N: 1P, 2N: 1P to 6N: 1P or 3N: 1P to 5N: 1P. The ratio of nucleotide (N) to phospholipid (P) can be in the range of, e.g., 1N: 1P, 2N: 1P, 3N: 1P. 4N: 1P, 5N: 1P, 6N: 1P, 7N: 1P, 8N: 1P, 9N: 1P, or 10N: 1P. Alternatively or additionally, the ratio of nucleotide (N) to phospholipid (P) is 4N: 1P.

General information on LNP compositions is provided in Liposome Technology, volumes I, II & III. (ed. Gregoriadis). Informa Healthcare, 2006; WO2012/006359, and WO2017/070620.

LNP delivery systems, and methods for their preparation are described in Geall et al. (2012) PNAS USA. Sep. 4; 109 (36): 14604-9 (LNP delivery system).

LNPs are typically 50 to 200 μm in diameter (Z-average). Suitably the LNPs have a polydispersity of 0.4 or less, such as 0.3 or less.

In one embodiment the carrier is a lipid nanoparticle (LNP).

The hydrophilic portion of a lipid can be PEGylated (i.e. modified by covalent attachment of a polyethylene glycol). This modification can increase stability and prevent non-specific adsorption of the liposomes. For instance, lipids can be conjugated to PEG using techniques such as those disclosed in Heyes et al. (2005) J Controlled Release 107:276-87 and WO2005/121348. Various lengths of PEG can be used e.g. between 0.5-8 kDa.

### Pharmaceutical Compositions

The RNA-LNP is formulated to maintain percent encapsulation of the RNA as compared to a control, and preferably a pre-lyophilized control, as well as a size and PDI comparable to the control. The composition may also comprise one or more excipients including primary sugars, and optionally, one or more secondary sugars, amino acids, and plasticizers as well as a suitable buffer to improve and/or maintain percent encapsulation during freeze-drying.

In certain embodiments, the RNA-LNP comprises a mRNA encapsulated in a lipid nanoparticle (LNP). For example, the mRNA may be a self-amplifying mRNA (SAM) encapsulated in a LNP, a SAM-LNP. Alternatively, the mRNA may be a non-replicating mRNA encapsulated in a LNP.

In general, the concentration of SAM-LNP may range from 60-250 μg/mL. In certain embodiments, SAM-LNP may be at a concentration of about 180 μg/mL or more, such as 180-220 μg/mL. 190-210 μg/mL, or preferably 60 μg/mL. In other embodiments, the concentration of SAM-LNP may range from 60-250 μg/mL. The lipid nanoparticle may comprise a cationic lipid, a zwitterionic lipid, and a cholesterol. The lipid nanoparticle may optionally comprise a PEGylated lipid. In certain embodiments, the lipid nanoparticle may comprise DSPC, DlinDMA, PEG-DMG and cholesterol. In other embodiments, the lipid nanoparticle may comprise DSPC, 2,5-bis((9Z,12Z)-octadeca-9,12-dien-1-yloxy)benzyl 4-(dimethylamino) butanoate) (“RV39”), PEG-DMG and cholesterol. See WO2016/037053, Example 1, describing RV39. In the figures, RV39 is represented as LKY 750.

In some embodiments, the cation-ionizable lipid is:

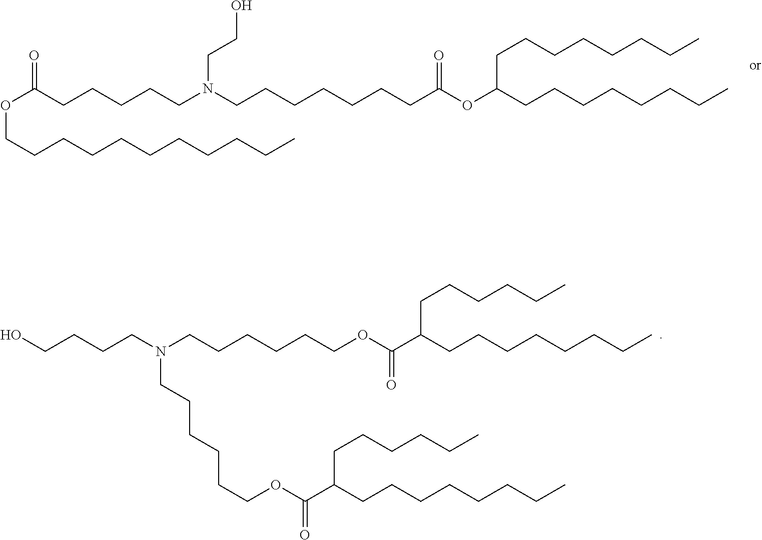 [SMILES: CCCCCCCCCCCOC(=O)CCCCCN(CCO)CCCCCCCC(=O)OC(CCCCCCCC)CCCCCCCC ; CCCCCCCCC(CCCCCC)C(=O)OCCCCCCN(CCCCO)CCCCCCOC(=O)C(CCCCCC)CCCCCCCC]

In some embodiments, the PEG-conjugated lipid comprises 2-[(polyethylene glycol)-2000]-N,N-ditetradecylacetamide or 1,2-dimyristoyl-rac-glycero-3-methoxypolyethylene glycol-2000. In 2-[(polyethylene glycol)-2000]-N,N-ditetradecylacetamide or 1,2-dimyristoyl-rac-glycero-3-methoxypolyethylene glycol-2000, the “2000” represents the median molecular weight in Daltons of the PEG. In some embodiments, the PEG-conjugated lipid comprises 1,2-dimyristoyl-sn-glycero-2-phosphoethanolamine-N-[methoxy (polyethylene glycol)]. In some embodiments, the PEG-conjugated lipid comprises 1,2-dimyristoyl-rac-glycerol-3-methoxypolyethylene glycol. In some embodiments, the PEG-conjugated lipid comprises:

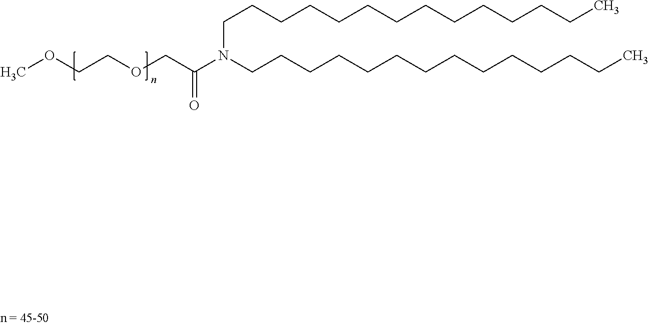

In certain embodiments, a pharmaceutical composition formulated for lyophilization comprises an RNA-LNP (e.g., wherein the RNA is mRNA, a self-amplifying mRNA (SAM), a non-replicating mRNA, etc.) and a primary sugar, such as sucrose, in a buffer. The primary sugar may be about 5 to 30%, 5 to 20%, 5 to 10%, or 7.5% (w/v) sucrose. In some aspects, the primary sugar may be 7.5% (w/v). In aspects, the pharmaceutical composition may be formulated in a buffer, such as Tris buffer. The buffer may comprise 20 mM Tris and 5 mM NaCl at a physiological pH of about 7 to 9 (e.g., pH 8.0). The composition may optionally include a plasticizer, such as sorbitol, glycerol or ethylene glycol in an amount of about 0.1 to 1.0% (w/v) (e.g., about 0.5% (w/v)).

In certain embodiments, a pharmaceutical composition formulated for lyophilization comprises an RNA-LNP (e.g., wherein the RNA is mRNA, a self-amplifying mRNA (SAM), a non-replicating mRNA, etc.) and a primary sugar, such as sucrose, in a buffer. The primary sugar may be about 5 to 30%, 5 to 20%, 5 to 10%, or 7.5% (w/v) sucrose. The pharmaceutical composition may be formulated in a buffer such as histidine buffer. The buffer may be about 20 mM histidine buffer. The pharmaceutical composition with sucrose and 20 mM histidine buffer may additionally and/or optionally comprise a secondary sugar, an amino acid, and/or a plasticizer. The secondary sugar may be selected from the group consisting of: glucose, trehalose, maltose or melezitose and may be present in an amount of about 0.1-5.0% (w/v) (e.g., 2.5% (w/v)). The amino acid may be selected from the group consisting of: arginine, methionine, histidine, lysine and alanine. The plasticizer may be selected from the group consisting of: glycerol, ethylene glycol or sorbitol. In aspects, the amino acid is present in an amount of about 0.1-1.0% (w/v) (e.g., 0.5% (w/v)), and the plasticizer is present in an amount of about 0.1-1.0% (w/v) (e.g., 0.5% (w/v)). In aspects, the primary sugar is sucrose, the amino acid is methionine, and the plasticizer is glycerol.

In another embodiment, a pharmaceutical composition formulated for lyophilization comprises an RNA-LNP (e.g., wherein the RNA is mRNA, a self-amplifying mRNA (SAM), a non-replicating mRNA, etc.), a primary sugar, an optional secondary sugar, one or more amino acids, and a plasticizer. In aspects, the RNA-LNP is a SAM-LNP. In aspects, the pharmaceutical composition comprises:
(a) a primary sugar such as sucrose;
(b) optionally, a secondary sugar such as glucose, trehalose, maltose or melezitose;
(c) one or more amino acids selected from the group consisting of arginine, methionine, histidine, lysine and alanine; and
(d) optionally, a plasticizer such as glycerol, ethylene glycol or sorbitol.

In certain embodiments, sucrose is present in an amount of about 5-30%, about 5-20%, about 5-10%, or about 7.5% (w/v). Methionine is present in an amount of about 0.1-1.0% or about 0.25-0.75% (w/v), or about 0.5% (w/v), and glycerol is present in an amount of about 0.1-1.0% or about 0.25-0.75% (w/v), or about 0.5% (w/v).

In certain embodiments, the primary sugar is sucrose, the amino acid is methionine, and the plasticizer is glycerol. The composition may comprise SAM-LNP, a primary sugar such as sucrose in an amount of about 7.5% (w/v), an amino acid such as methionine in an amount of about 0.5% (w/v), and a plasticizer such as glycerol in an amount of about 0.5% (w/v).

In certain embodiments, the composition may comprise SAM-LNP, a primary sugar such as sucrose in an amount of about 5% (w/v), a secondary sugar such as trehalose in an amount of about 2.5% (w/v), an amino acid such as methionine in an amount of about 0.5% (w/v), and a plasticizer such as glycerol in an amount of about 0.5% (w/v).

In certain embodiments, the primary sugar may be present in an amount of about 5-10% (w/v), preferably about 7.5% (w/v). Both the primary sugar and secondary sugar may be present, with the primary sugar present in an amount of about 5-10% (w/v), and preferably about 5.0% (w/v); and the secondary sugar may be present in an amount of about 0.1-5% (w/v), and preferably about 2.5% (w/v). The one or more amino acids may be present in an amount of about 0.1-1.0% (w/v), and preferably about 0.5% (w/v). The plasticizer may be present in an amount of about 0.1-1.0% (w/v), and preferably about 0.5% (w/v).

In certain embodiments, the pharmaceutical composition does not include dextran, PVP, Tween 20, or P-188. More specifically, the pharmaceutical composition does not include 0.1% dextran, 1% PVP, 1% Tween 20, or 1% P188.

In still other aspects, the LNP lipid composition includes an amount of polyethylene glycol that is between about 1-2%.

Thus, the invention provides a pharmaceutical composition formulated for lyophilization of lipid nanoparticle encapsulated RNA (RNA-LNP), along with kits and vaccines. In aspects, the RNA comprises an mRNA encapsulated in a lipid nanoparticle (LNP). In aspects, the mRNA-LNP comprises a self-amplifying mRNA (SAM) encapsulated in a lipid nanoparticle (LNP) or SAM-LNP. In aspects, the mRNA-LNP comprises a mRNA encapsulated in a lipid nanoparticle (LNP) that is not self-amplifying (e.g., a non-replicating mRNA). The composition is designed to maintain the stability of the LNP encapsulated RNA during the lyophilization process and to allow storage under standard refrigeration temperatures or at room temperature.

A novel lyophilization process is presented, for the compositions provided herein, for development of a thermostable composition (e.g., for a vaccine or drug product) to eliminate the need for cold chain storage by allowing storage at 5° C. up to room temperature, thus making stockpiling and distribution of said compositions feasible. However, when available and practical, the lyophilized composition can be stored at standard refrigeration temperatures above 0° C. and below room temperature which is usually considered to be 20, 21 or 22° C. For maximum storage stability, the lyophilized composition may be stored at temperatures just above 0° C. to up to 5° C. preferably just above 0° C. to up to 4° C., and more preferably 1° C. to 4° C. After lyophilization, the lyophilized composition will be stored for a period of time to allow for distribution to a site where the vaccine will be reconstituted and administered. Therefore, considering that the vaccine may be need to be stockpiled for future use and that there may be some delays in distribution and administration, it may be necessary to store the vaccine for up to one year or longer, but the vaccine will preferably be stored for a shorter time such as 9 months, 6 months, 3 months or less.

As referenced herein, a primary sugar refers to a sugar present in the pharmaceutical composition in an amount of about 5% (w/v) or greater. Typically, the primary sugar may be present in an amount of 5-30% (w/v), about 5-20% (w/v), about 5-10% (w/v), or about 7.5% (w/v). In certain embodiments, the primary sugar is sucrose.

As referenced herein, a secondary sugar refers to a sugar present in the pharmaceutical composition in an amount from 0.1% to 5% (w/v) or about 2.5% (w/v). In certain embodiments, the secondary sugar is glucose, trehalose, maltose, or melezitose. A secondary sugar is in addition to the primary sugar, and in certain embodiments, is optional.

An amino acid refers to any of the naturally occurring amino acids or synthesized amino acids. In certain embodiments, the amino acid is arginine, methionine, histidine, lysine or alanine. In certain embodiments, the amino acid is present in an amount from about 0.1% to 1.0% (w/v) or about 0.5% (w/v).

As referenced herein, a plasticizer refers to a component that lowers the glass transition temperature (Tg), thereby increasing molecular mobility of the LNP. In aspects, plasticizers may be small molecules, such as glycerol or sorbitol or ethylene glycol, that help fill small open volumes arising from larger molecules. In certain embodiments, the plasticizer is present in an amount from about 0.1% to 1.0% (w/v) or about 0.5% (w/v).

Any suitable buffer may be used with the embodiments provided herein. Suitable buffers include but are not limited to Tris, histidine, phosphate, citrate, and HEPES buffer. Buffers may be at any suitable concentration including 5 mM-20 mM and may have any suitable pH ranging from 6 to 9.5.

Formulations with a Primary Sugar

In an embodiment, the pharmaceutical composition may comprise RNA-LNP (e.g., mRNA, SAM-LNP) formulated with about 5% (w/v) or greater of a primary sugar such as sucrose. For example, the primary sugar may be present in an amount of about 5-30% (w/v), about 5-20% (w/v), about 5-15% (w/v), about 5-10% (w/v), or about 7.5% (w/v). For example, the composition may comprise RNA-LNP formulated with sucrose in Tris buffer with NaCl at physiological pH (e.g., 7.5-9 pH). In aspects, the formulation may comprise RNA-LNP formulated with 7.5% (w/v) sucrose in 20 mM Tris and 5 mM NaCl buffer at physiological pH (e.g., pH 8.0).

In another aspect, an optional plasticizer may be present in an amount of about 0.1-1% (w/v), and preferably about 0.5% (w/v).

In aspects, the composition may comprise any suitable buffer, including Tris, phosphate, citrate, HEPES and histidine buffer at any suitable concentration (e.g., about 5 mM-30 mM) and suitable pH (e.g., about 6-9.5).

In aspects, SAM-LNP may be at a concentration for lyophilization of about 60-250 μg/mL or more, such as about 60-220 μg/mL, about 60-180 μg/mL, about 60-150 μg/mL, about 60-120 μg/mL, about 60-90 μg/mL or preferably about 60 μg/mL, about 90 μg/mL, about 120 μg/mL, about 150 μg/mL, about 180 μg/mL or about 200 μg/mL. The concentration refers to ug of SAM in SAM-LNP per mL of the dose (e.g., vaccine). The LNP may comprise a cationic lipid, a zwitterionic lipid, and a cholesterol. The lipid nanoparticle may optionally comprise a PEGylated lipid. In some aspects, the lipid nanoparticle may comprise or consist of DSPC, DlinDMA, PEG-DMG and cholesterol. In certain embodiments, the lipid nanoparticle may comprise or consist of DSPC. RV39, PEG and cholesterol.

Formulations with a Primary Sugar, Secondary Sugar, Amino Acid and Plasticizer

In aspects, the composition may comprise RNA-LNP, a primary sugar, a secondary sugar (optional), an amino acid, and a plasticizer.

SAM-LNP may be present at a concentration of about 60 μg/mL or more, such as about 60-250 μg/mL, such as about 60-220 μg/mL, about 60-180 μg/mL, about 60-150 μg/mL, about 60-120 μg/mL, about 60-90 μg/mL or preferably about 60 μg/mL, about 90 μg/mL, about 120 μg/mL, about 150 μg/mL, about 180 μg/mL or about 200 μg/mL. The LNP may comprise a cationic lipid, a zwitterionic lipid, and a cholesterol. The lipid nanoparticle may optionally comprise a PEGylated lipid. In some aspects, the lipid nanoparticle may comprise or consist of DSPC, DlinDMA, PEG-DMG and cholesterol. In certain embodiments, the lipid nanoparticle may comprise or consist of DSPC. RV39, PEG and cholesterol.

In aspects, the pharmaceutical composition may comprise RNA-LNP with:
(a) a primary sugar such as sucrose;
(b) optionally, a secondary sugar such as glucose, trehalose, maltose or melezitose;
(c) one or more amino acids selected from the group consisting of arginine, methionine, histidine, lysine and alanine; and
(d) a plasticizer such as glycerol or sorbitol.

The primary sugar may be present in an amount of 5-30% (w/v), about 5-20% (w/v), about 5-15% (w/v), about 5-10% (w/v), or about 7.5% (w/v). For example, the composition may comprise RNA-LNP formulated with sucrose in a buffer.

In certain embodiments, the pharmaceutical composition comprises a primary sugar (e.g., sucrose) at a concentration of about 5-10% (w/v) and preferably about 7.5% (w/v); and a secondary sugar (e.g., trehalose, glucose, maltose, or melezitose) at a concentration of about 0-5% (w/v) and preferably about 2.5% (w/v).

In aspects, the one or more amino acids are selected from the group consisting of arginine, lysine, histidine, methionine, alanine. The one or more amino acids may be present in an amount of about 0.1-1.0% (w/v), and preferably about 0.5% (w/v).

In aspects, the plasticizer is selected from the group consisting of sorbitol, glycerol, and ethylene glycol. In another aspect, the plasticizer may be present in an amount of about 0.1-1% (w/v), and preferably about 0.5% (w/v).

Formulations may include any suitable buffer including Tris (pH of 7.5-9.5), HEPES (pH of 7-9.0), phosphate (pH of 7-8.5), histidine (pH 6-7) or citrate buffer (pH of 6-7). In some aspects, suitable buffers include a range of 7.1-9.1, a pKa of 8.07 at room temperature, and have a percentage encapsulation up to ≤80% post lyophilization. Any suitable buffer concentration may be used (e.g., 5-30 mM). In certain embodiments, the formulation may comprise 20 mM Tris and 5 mM NaCl buffer at physiological pH (e.g., pH 8.0).

In aspects, the primary sugar is sucrose, the amino acid is methionine, and the plasticizer is glycerol. In certain embodiments, sucrose is present in an amount of about 5-10% (w/v) or about 7.5% (w/v), the secondary sugar is present in an amount of 2.5% (w/v), methionine is present in an amount of about 0.1-1.0% or about 0.25-0.75% (w/v), and glycerol is present in an amount of about 0.1-1% or about 0.25-0.75% (w/v) or about 0.5% (w/v).

In aspects, the composition comprises RNA-LNP (e.g., SAM-LNP), sucrose, trehalose, methionine, and glycerol. For example, the composition comprises a primary sugar such as sucrose in an amount of about 5% or 7.5% (w/v), a secondary sugar in an amount of about 2.5% (w/v), an amino acid such as methionine in an amount of about 0.5% (w/v), and a plasticizer such as glycerol in an amount of about 0.5% (w/v). As another example, sucrose is present in an amount of about 1-10% or about 5-10% (w/v), trehalose is present in an amount of about 0-5% (w/v), methionine is present in an amount of about 0.1-1.0% or about 0.25-0.75% (w/v), and glycerol is present in an amount of about 0.1-1% or about 0.25-0.75% (w/v). In still other aspects, the composition comprises SAM-LNP, a primary sugar such as sucrose in an amount of about 5% (w/v), a secondary sugar such as trehalose in an amount of about 2.5% (w/v), an amino acid such as methionine in an amount of about 0.5% (w/v), and a plasticizer such as glycerol in an amount of about 0.5% (w/v).

Formulations may include any suitable buffer including Tris (pH of 7.5-9.5), HEPES (pH of 7-9.0), phosphate (pH of 7-8.5), histidine (pH 6-7) or citrate buffer (pH of 6-7). In some aspects, suitable buffers include a range of 7.1-9.1, a pKa of 8.07 at room temperature, and have a percentage encapsulation up to ≤80% post lyophilization. Any suitable buffer concentration may be used (e.g., 5-30 or 5-20 mM of any suitable buffer).

Formulations with a Primary Sugar and Histidine Buffer

In certain embodiments, the pharmaceutical composition may comprise RNA-LNP (e.g., wherein the RNA is mRNA, and the mRNA is a self-amplifying mRNA (SAM) or a non-replicating mRNA) formulated with about 5-10% sucrose in Histidine buffer. For example, the pharmaceutical composition may comprise RNA-LNP formulated about with 5-10% sucrose in 5 mM, 10 mM or 20 mM or 30 mM Histidine buffer at a pH of 6-7. In aspects, formulations may comprise mRNA (e.g., SAM-LNP DP), with sucrose (7.5% (w/v)), and 20 mM Histidine buffer at a pH from 6-7. Any suitable concentration (e.g., about 5-30 or 5-20 mM) of histidine buffer may be used.

The pharmaceutical composition with sucrose and histidine buffer may additionally and/or optionally comprise one or more of a secondary sugar, an amino acid, and a plasticizer. The secondary sugar may be selected from the group consisting of: glucose, trehalose, maltose or melezitose. The secondary sugar may be present in an amount of about 0-5% (w/v) or about 2.5% (w/v). The amino acid may be selected from the group consisting of: arginine, methionine, histidine, lysine and alanine. The amino acid may be present in an amount of about 0.1-1.0% (w/v) or about 0.5% (w/v). The plasticizer may be selected from the group consisting of: glycerol or sorbitol or ethylene glycol. The plasticizer may be present in an amount of about 0.1-1% (w/v) or about 0.5% (w/v).

In certain embodiments, the primary sugar is sucrose, the amino acid is methionine, and the plasticizer is glycerol. For example, the primary sugar is sucrose 7.5% (w/v), the amino acid is methionine (e.g., at about 0.5% or 1.0% (w/v)), and the plasticizer is glycerol (e.g., at 0.5% or 1.0% (w/v)).

### Combinations

It is contemplated herein that sucrose concentrations greater than or equal to 7.5%, in combination with one or more amino acids, buffers such as histidine, and in combination with the optimized lyophilization processes provided herein, will lead to further improvements in percent encapsulation, particle size, and PDI while retaining efficacy.

Pharmaceutical compositions provided herein (e.g., SAM-LNP about 60-250 μg/mL in 20 mM Tris, 5 mM NaCl and 7.5% sucrose) may be formulated with sterile water or with any suitable buffer. Buffers include but are not limited to citrate buffer with a pH of 6 to 7, histidine buffer with a pH of 6 to 7, phosphate buffer with a pH of 6.5 to 8.5, and HEPES buffer with a pH of 6.5 to 9. Buffer salts will typically be included in the 5-30 or 2-20 mM range. Pharmaceutical compositions of the invention may have a pH between 5.0 and 9.5, or between 6.0 and 9 or any other suitable range.

In other aspects, the pharmaceutical composition does not include dextran, PVP, Tween 20, or P-188. In other aspects, the pharmaceutical composition does not include 0.1% dextran, 1% PVP, 1% Tween 20, or 1% P188.

The compositions provided herein maintain and/or improve percent encapsulation comparable to a control formulation.

For the compositions provided herein, in certain embodiments, sucrose is present in an amount greater than or equal to 5% (w/v) or preferably 7.5% (w/v). Additionally plasticizers are present in an amount of about 0.5% (w/v).

RNA will be administered as a component in a pharmaceutical composition for immunising subjects against various diseases. These compositions will typically include a pharmaceutically acceptable carrier in addition to the RNA, often as part of a delivery system as described above. A thorough discussion of pharmaceutically acceptable carriers is available in Gennaro (2000) Remington: The Science and Practice of Pharmacy. 20th edition, ISBN: 0683306472.

A pharmaceutical composition of the invention may include one or more small molecule immunopotentiators. For example, the composition may include a TLR2 agonist (e.g. Pam3CSK4), a TLR4 agonist (e.g. an aminoalkyl glucosaminide phosphate, such as E6020), a TLR7 agonist (e.g. imiquimod), a TLR8 agonist (e.g. resiquimod) and/or a TLR9 agonist (e.g. IC31). Any such agonist ideally has a molecular weight of <2000 Da. Where an RNA is encapsulated, in some embodiments such agonist(s) are also encapsulated with the RNA, but in other embodiments they are unencapsulated. Where an RNA is adsorbed to a particle, in some embodiments such agonist(s) are also adsorbed with the RNA, but in other embodiments they are unadsorbed.

Compositions of the invention may include metal ion chelators. These can prolong RNA stability by removing ions which can accelerate phosphodiester hydrolysis. Thus a composition may include one or more of EDTA, EGTA, BAPTA, pentetic acid, etc. Such chelators are typically present at between 10-500 μM e.g. 0.1 mM. A citrate salt, such as sodium citrate, can also act as a chelator, while advantageously also providing buffering activity.

Pharmaceutical compositions of the invention may have an osmolality of between 200 mOsm/kg and 400 mOsm/kg, e.g. between 240-360 mOsm/kg, or between 290-310 mOsm/kg.

Pharmaceutical compositions of the invention may include one or more preservatives, such as thiomersal or 2-phenoxyethanol. Mercury-free compositions are preferred, and preservative-free vaccines can be prepared.

Pharmaceutical compositions of the invention are preferably sterile.

Pharmaceutical compositions of the invention are preferably non-pyrogenic e.g. containing <1 EU (endotoxin unit, a standard measure) per dose, and preferably <0.1 EU per dose.

Pharmaceutical compositions of the invention are preferably gluten free.

Pharmaceutical compositions of the invention may be prepared in unit dose form. In some embodiments a unit dose may have a volume of between 0.1-1.0 ml e.g. about 0.5 ml.

The compositions may be prepared as injectables, either as solutions or suspensions. Injectables for intramuscular administration are typical. Compositions may be lyophilized and reconstituted prior to administration.

Compositions comprise an immunologically effective amount of RNA, as well as any other components, as needed. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. The RNA content of compositions of the invention will generally be expressed in terms of the amount of RNA per dose. In embodiments, a preferred dose has <10 ug RNA, and expression can be seen at much lower levels e.g. ≤1 μg/dose, ≤100 ng/dose, <10 ng/dose, <1 ng/dose, etc.

The invention also provides a delivery device (e.g. syringe, etc.) containing a pharmaceutical composition of the invention. This device can be used to administer the composition to a vertebrate subject.

RNAs are not delivered in combination with ribosomes and so pharmaceutical compositions of the invention are ribosome-free.

### Lyophilization

A novel lyophilization process is presented, for the compositions provided herein, for development of a thermostable composition (e.g., for a vaccine or drug product) to eliminate the need for cold chain storage by allowing storage at 5° C. up to room temperature, thus making stockpiling and distribution of said compositions feasible.

The lyophilization process comprises freezing, primary drying, and secondary drying steps. For each process step, a cycle time, temperature, and pressure is selected along with a ramp time to achieve and maintain a desired temperature and pressure of the lyophilization chamber containing the pharmaceutical compositions for that process step.

In aspects, the lyophilization process may be used with the formulations provided herein, for example, a product containing liposome encapsulated mRNA, such as SAM-LNP or non-replicating mRNA.

The formulation is designed to preserve the percent encapsulation of the mRNA. Ideally, the formulation is designed to maintain a percent encapsulation of mRNA that is within 10%, 5%, 2% or better than a liquid control that does not undergo lyophilization. During the lyophilization process, samples may be obtained of the pharmaceutical composition, and may be characterized in terms of size, PDI and percent encapsulation as well as efficacy. Any suitable lyophilization device may be used for performing lyophilization of the compositions provided herein.

A sample loading step is performed to load the pharmaceutical composition into a lyophilization chamber. A first thermal equilibrium step is performed with a ramp rate of 1° C. per minute to reach a temperature of 5° C. for a duration of about 0.5 hours. Once completing the first thermal equilibrium step, a second thermal equilibrium step is performed. The second thermal equilibrium step is performed with a ramp rate of 1° C. per minute to reach a temperature of about minus 5° C. for a duration of about 0.5 hours.

### Freezing

The composition, which is in a solution at room temperature, is subjected to primary freezing. Typically, primary freezing is performed at about minus 40° C., with a freezing rate ranging from about 0.1° C./min to about 1.0° C./min. Once reaching the desired freezing temperature, the lyophilization chamber may be held at this temperature for one hour or more.

### Primary Drying

After freezing, the frozen composition may undergo a primary drying step. Primary drying removes moisture from the frozen sample. Conditions for primary drying may include a temperature ranging from minus 25° C. to minus 35° C. (preferably at about minus 29 or about minus 30° C.), held at the designated temperature for 25 or more hours at a pressure of about 55-60 mTorr. Ramp rates (freezing rates) during primary drying may range from 0.1° C./min up to 1.0° C./min, e.g., 0.5° C./min. In certain embodiments, the primary drying step may comprise a temperature of minus 29° C. for 27 hours at a vacuum pressure of 57 mTorr.

### Secondary Drying

After primary drying, the composition may undergo a secondary drying step. Secondary drying removes additional moisture from the product of primary drying. Conditions for secondary drying may include a temperature ranging from 0° C. to 25° C. or higher, held at the designated temperature for 6-48 hours or more at a pressure of about 55-60 mTorr. The ramp rate may be about 0.1° C./min.

In aspects, the secondary drying temperature may range from 5 to 40° C. and may include a drying duration time of 4 hours to 48 hours. In some aspects, the secondary drying temperature may be 5° C. for about 48 hours. In another aspect, the secondary drying temperature may be about 15° C. for about 12 hours. In another aspect, the secondary drying temperature may be about 25° C. for about 6 hours. In another aspect, the secondary drying temperature may be about 40° C. for about 4 hours. In aspects, the vacuum pressure during secondary drying is 60 mTorr and the ramp rate is about 0.1° C./min.

In certain embodiments, a secondary drying temperature of 5° C. for about 48 hours leads to about a 10% entrapment drop. A secondary drying temperature of about 15° C. for about 12 hours leads to about a 14% entrapment drop. As another example, a 25° C. secondary drying temperature for 6 hours leads to about a 17% entrapment drop. Accordingly, a temperature range of 5-15° C. for 12-48 hours minimizes the drop in percentage entrapment.

It was surprisingly observed that the percent entrapment initially decreased during secondary drying but returned toward its initial value by the end of secondary drying. It was also observed that the particle size initially increased during secondary drying but returned towards its initial value by the end of secondary drying. Additionally, it was observed that potency was initially reduced during secondary drying but returned towards its initial value by the end of secondary drying.

In an embodiment, a method of lyophilization for the pharmaceutical compositions provided herein comprises an initial freezing step, followed by drying, which can include a primary drying step, followed by a secondary drying step. The initial freezing step may comprise placing a pharmaceutical composition into a lyophilization chamber; and subjecting the composition to an initial freezing step that comprises decreasing the temperature of the lyophilization chamber from an initial temperature to a freezing temperature of about −39° C. or lower, at a controlled freezing ramp rate and holding the chamber at the freezing temperature to convert water to ice. The freezing step ideally converts all of the water to ice. This freezing step is followed by a drying step, which can include at least a primary drying step and a secondary drying step, to remove any moisture from the composition to form a lyophilized composition.

Lyophilization, as to be described below, includes an initial sample loading step and a subsequent thermal equilibration step (which may be include two separate steps with different conditions), followed by two main steps: (1) freezing and (2) drying (which can include a separate primary drying step and a subsequent secondary drying).

A loading step is performed in which the lyophilization formulation, at room temperature, is loaded into the lyophilization chamber. Next, one or two or more than two thermal equilibration steps are performed wherein the temperature of the lyophilization chamber is reduced from the sample loading temperature (often room temperature) to a temperature between the sample loading temperature and the freezing temperature.

For the freezing step, the temperature of the lyophilization formulation is subjected to a temperature or temperatures well below the freezing point of the product, generally for a few hours, until completely frozen to form ice and crystallize out any solutes. During this step the temperature is typically reduced at a set ramp rate in terms of ° C./minute and then held at the target temperature for the remainder of the freezing step. This step can be performed at various convenient pressures but typically is performed at atmospheric pressure.

Once the freezing step is completed, drying step, which may include multiple drying steps such as primary and secondary drying steps, is conducted. The drying step is conducted at reduced pressure (below atmospheric pressure) and the atmosphere inside the lyophilization chamber is continuously monitored for the presence of water.

Like thermal equilibration, drying can be performed in two or more distinct steps where the conditions are changed between steps (see, for example, Tables 5, 6, 15, 17 and 18 below) or it can be performed in one step (see, for example, Table 19 below). Two separate steps can be used when it is difficult to remove all or substantially all of the water molecules under one set of conditions while maintaining product quality. In such a situation, a secondary drying step can performed after a primary drying step. Regardless of whether one or more than one drying steps are performed, drying removes any water in the sample by sublimation wherein water converts directly from a solid to gas. In other words, the drying step is a step in which a sublimation process removes unbound water from the composition. The primary drying step is followed by a secondary drying step in which bound water molecules are removed. At the end of the drying process, which is after the secondary drying step if two drying steps are used, the container is filled with inert gas and stoppered. This conversion from a solid form directly to gas occurs even if the drying temperature is above freezing because the sample itself remains below freezing until drying is complete. When water is no longer detected by sensors that sample the atmosphere in the chamber at a particular point in time under given conditions, this is an indication that the particular step is completed and the conditions such as pressure and/or temperature can be changed. In particular, the temperature may be raised for the next step.

Two different methods are disclosed below for preparing lyophilized compositions which are referred to in this application as a “lower drying temperature method” and a “higher drying temperature method”. One advantage of the higher drying temperature method is that the total time of the method can be shortened. Even though the initial freezing step might take longer because the compositions are taken to a lower temperature, any lengthening of the freezing time can be more than offset by shortening of the drying time by performing drying at a higher temperature (which can result in a shortened time for drying). Thus, any lengthening of time in the initial freezing step can be more than offset by a shortening of the drying step. In particular, the temperature of the primary drying step can be shortened thereby reducing the total time of the drying step.

### General Conditions for Lyophilization

The lyophilization process of the present invention, in one embodiment, will typically start with a liquid formulation of LNPs containing RNA at any suitable initial temperature. The liquid formulation of LNPs containing RNA (any type of RNA discussed in this patent application, including self-amplifying mRNA or SAM), before beginning the lyophilizaton process, will be at any suitable or convenient initial temperature. This initial temperature before initial thermal equilibration and freezing occur, is typically at room temperature (about 20-22° C.) or at room temperature down to a typical refrigerated (about 2 to about 8° C.) temperature, such as just above a freezing. The sample loading, thermal equilibration and freezing steps (described in more detail below) are typically conducted at atmospheric pressure.

Typically, a plurality of vials, each containing the same liquid formulation, will be lyophilized at the same time. In large batches, 100 or more vials are treated at the same time, such as 400 to 1,000 or 400-800 vials, depending upon the size of the lyophilization chamber. It is convenient for large batches to include multiple vials on each of multiple shelves in the lyophilization chamber. The vials are subjected to thermal equilibration and an initial freezing step followed by drying, which will typically include a primary drying step and a secondary drying step. During all parts of the process, after the samples reach freezing temperature, removal of water occurs by sublimation as it typical in freeze drying processes. Almost all (or all) of the water removal occurs during the drying steps, which are performed at reduced pressures. In the primary drying step, a sublimation process removes unbound water such as water that is present in the form of ice crystals from the composition. The primary drying step is followed by a secondary drying step in which bound water molecules are also removed by desorption. The drying is continued until there is no measurable water in the atmosphere of the chamber. At the end of drying, and in particular at the end of the secondary drying, the container is backfilled with inert gas and stoppered.

### Conditions for the Lower Drying Temperature Method

In certain embodiments (including, for example, the “lower drying temperature method”), the initial freezing step comprises reducing the temperature of the lyophilization chamber housing the pharmaceutical composition to about minus 40° C. Parameters of the initial freezing step include a cycle time of about 1 hour and a pressure of about 60 mTorr. The freezing ramp rate may range from about 0.1° C./min to about 1° C./min. Freezing may be done at atmospheric pressure or slightly below atmospheric (such as 900 to 600 mbars) in order to help seal the door of the lyophilizer to the drying chamber and avoid any leaks from the outside during primary or secondary drying. All steps before the drying steps can be performed at atmospheric pressure or slightly below atmospheric pressure, as mentioned above.

In certain embodiments, a primary drying step follows the freezing step and comprises raising the temperature of the lyophilization chamber housing the pharmaceutical composition to about minus 28 to about minus 31° C. or about minus 29 or about minus 30° C. In aspects, parameters of the primary drying step include a cycle time of about 25-32 hours (or about 27-30 hours or about 27 hours or about 30 hours) and a pressure of about 55-62 mTorr or about 57 60 mTorr or about 57 mTorr or about 60 mTorr. The ramp rate may range from about 0.1° C./min to about 1° C./min or may be about 0.5° C./min.

In certain embodiments, a secondary drying step follows the primary drying step and comprises raising the temperature of the lyophilization chamber housing the pharmaceutical composition to about 10 to 20° C., or about 15° C. Parameters of the secondary drying step include a cycle time of about 10-15 hours or about 12 hours and a pressure of about 55-62 mTorr or about 57-60 mTorr or about 57 mTorr. In certain embodiments, the ramp rate may be about 0.1° C./min.

In certain embodiments, a longer cycle time and lower temperature may be advantageous. In aspects, the secondary drying temperature is within a range of about 0-40° C., about 5-40° C., about 15-40° C., about 25-40° C., about 5-25° C., about 5-15° C., or about 5° C. or about 15° C. The pressure may be about 55-62 mTorr or about 57-60 mTorr or about 57 mTorr or about 60 mTorr. The secondary drying time may range from 4 to 48 hours. In certain embodiments, the secondary drying time is within a range of 2-6° C. degrees at about 36-60 hours, and preferably, about 48 hours at 4° C.

The lower drying temperature method described above usually takes at least 41 hours, and typically takes at least 49 hours, such as 49 to 53 hours, and more specifically about 50 hours under the conditions described in Example 2 in the Examples section below.

### Conditions for the Higher Drying Temperature Method

The pressures used in the higher drying temperature method can typically be below as the pressures in the corresponding steps in the lower drying temperature method. The main differences between the two processes are that (1) the freezing temperature is lowered and/or (2) the drying temperatures, particularly the primary drying temperature, is raised.

In certain embodiments (including, for example, the “higher drying temperature method”), the initial freezing step comprises reducing the temperature of the lyophilization chamber housing the pharmaceutical composition to below any observed energy transition temperature, preferably at least 2° C. below the energy transition temperature, preferably 2 to 30° C. below any energy transition temperature. Even if no energy transition temperature is observed that is not due to the presence of sucrose, this higher drying temperature method can also be used. Thus, in a more general sense, the higher drying temperature process can start with an initial freezing temperature of less than about −40° C., such as less than −40° C. or less than −45° C. or less than −50° C. In one embodiment, the freezing temperature can be −40° C. to −75° C. or −45° C. to −75° C. or −50 to −70° C. Any necessary adjustments in the thermal equilibrium and freezing ramp rates can also be made. Parameters of the initial freezing step may include a cycle time of about 1 hour and a pressure of about 60 mTorr. The freezing ramp rate may be any suitable ramp rate such as a range from about 0.1° C./min to about 1° C./min.

In certain embodiments (in particular, in the embodiments of the higher drying temperature method), a primary drying step follows the freezing step and comprises raising the temperature of the shelves of the lyophilization chamber housing the pharmaceutical composition to a temperature above about minus 30° C. The conditions of the primary drying step can be a temperature of above 0° C. such as 0 to 10° C. or 0 to 15° C. . . . In certain aspects, parameters of the primary drying step include a cycle time of less than 32 hours, such as between about 10-15 hours (or about 12.5 hours or about 13.5 hours) and a pressure of about 20-62 mTorr or 22-62 m Torr or 40-62 mTorr, such as 55-62 mTorr or about 57-60 mTorr or about 57 mTorr or about 60 mTorr. The ramp rate may range from about 0.1° C./min to about or 1.5° C./min. or about 0.1° C./min to about or 1.0° C./min or may be about 0.5° C./min. or about 0.1° C./min to about 1° C./min or may be about 0.5 or 1.0 or 1.5° C./min.

In certain embodiments, a secondary drying step follows the primary drying step and comprises raising the temperature of the lyophilization chamber housing the pharmaceutical composition to about 10 to 20° C., or about 15° C. Parameters of the secondary drying step include a cycle time of about 10-15 hours or about 12 hours and a pressure of about 20-62 mTorr or 22-62 m Torr or 40-62 mTorr, about 55-62 mTorr or about 57-60 mTorr or about 57 mTorr. In certain embodiments, the ramp rate may be about 0.1° C./min.

In certain embodiments, a longer cycle time and lower temperature may be advantageous. In aspects, the secondary drying temperature is within a range of about 0-40° C., about 5-40° C., about 15-40° C., about 25-40° C. about 5-25° C., about 5-15° C., or about 5° C. or about 15° C. The pressure may be about 55-62 mTorr or about 57-60 mTorr or about 57 mTorr or about 60 mTorr. The secondary drying time may range from 4 to 48 hours. In certain embodiments, the secondary drying time is within a range of 2-6° C. degrees at about 36-60 hours, and preferably, about 48 hours at 4° C.

Overall, the higher drying temperature method can be completed faster than the lower drying temperature method, mainly due to a reduction in the drying time. For example, the lower drying temperature method can be completed in less than 40 total hours, such as about 30 to 40 hours, more specifically about 35 hours under the conditions described in Example 3 in the Examples section below.

### Combinations of Formulations and Lyophilization

A pharmaceutical composition formulated for stable lyophilization of lipid nanoparticle encapsulated RNA comprising:
lipid nanoparticle encapsulated RNA (RNA-LNPs, mRNA-LNPs) at a concentration ranging from about 60 μg/mL to 250 μg/mL, wherein the lipid nanoparticle comprises a cationic lipid (e.g., RV39, DlinDMA, etc.), a zwitterionic lipid (e.g., DSPC, etc.), a cholesterol, and a PEG, and
wherein the pharmaceutical composition comprises:
   sucrose in an amount of at least 5% (w/v), for example, 7.5% (w/v); 20 mM Tris buffer with 5 mM NaCl;
   optionally, a plasticizer selected from the group consisting of sorbitol or glycerol in an amount of 0.25%-1.0% (w/v), and preferably about 0.5% (w/v); and
   optionally, an amino acid selected from the group consisting of methionine, histidine, and arginine in an amount of 0.25%-1.0% (w/v), and preferably about 0.5% (w/v).

A pharmaceutical composition formulated for stable lyophilization of lipid nanoparticle encapsulated RNA comprising:
lipid nanoparticle encapsulated RNA (RNA-LNPs, mRNA-LNPs) at a concentration ranging from about 60 μg/mL to 250 μg/mL, wherein the lipid nanoparticle comprises a cationic lipid (e.g., RV39, DlinDMA, etc.), a zwitterionic lipid (e.g., DSPC, etc.), a cholesterol, and a PEG, and
wherein the pharmaceutical composition comprises:
   sucrose in an amount of at least 5% (w/v), for example, 7.5% (w/v);
   a buffer that is a 20 to 30 mM histidine buffer with a pH from 6 to 6.5;
   optionally, a plasticizer selected from the group consisting of sorbitol or glycerol in an amount of 0.25%-1.0% (w/v), and preferably about 0.5% (w/v); and
   optionally, an amino acid selected from the group consisting of methionine, histidine, and arginine in an amount of 0.25%-1.0% (w/v), and preferably about 0.5% (w/v).

A pharmaceutical composition formulated for stable lyophilization of lipid nanoparticle encapsulated RNA comprising:
lipid nanoparticle encapsulated RNA (RNA-LNPs, mRNA-LNPs) at a concentration ranging from about 60 μg/mL to 250 μg/mL, wherein the lipid nanoparticle comprises a cationic lipid (e.g., RV39, DlinDMA, etc.), a zwitterionic lipid (e.g., DSPC, etc.), a cholesterol, and a PEG, and
wherein the pharmaceutical composition comprises:
   sucrose in an amount of 20% (w/v); and
   optionally, a plasticizer selected from the group consisting of sorbitol or glycerol in an amount of 0.25%-1.0% (w/v), and preferably about 0.5% (w/v); and
   optionally, an amino acid selected from the group consisting of methionine, histidine, and arginine in an amount of 0.25%-1.0% (w/v), and preferably about 0.5% (w/v); and
   wherein the buffer comprises a 20 mM Tris buffer with 5 mM NaCl or a 20 to 30 mM histidine buffer at a pH from 6 to 6.5.

A pharmaceutical composition formulated for stable lyophilization of lipid nanoparticle encapsulated RNA comprising:
lipid nanoparticle encapsulated RNA (RNA-LNPs, mRNA-LNPs) at a concentration ranging from about 60 μg/mL to 250 μg/mL, wherein the lipid nanoparticle comprises a cationic lipid (e.g., RV39, DlinDMA, etc.), a zwitterionic lipid (e.g., DSPC, etc.), a cholesterol, and a PEG, and
wherein the pharmaceutical composition comprises:
   sucrose in an amount of about 5% (w/v);
   a secondary sugar selected from the group consisting of trehalose, glucose, stachyose, or maltose in an amount of about 2.5% (w/v),
   optionally, a plasticizer selected from the group consisting of sorbitol or glycerol in an amount of 0.25%-1.0% (w/v), and preferably about 0.5% (w/v); and
   optionally, an amino acid selected from the group consisting of methionine, histidine, and arginine in an amount of 0.25%-1.0% (w/v), and preferably about 0.5% (w/v).
   wherein the buffer comprises a 20 mM Tris buffer with 5 mM NaCl or a 20 to 30 mM histidine buffer at a pH from 6 to 6.5.

A vaccine comprising the pharmaceutical composition, wherein the pharmaceutical composition is lyophilized according to a lyophilization process comprising:
subjecting the composition to an initial freezing step that comprises decreasing the temperature of the lyophilization chamber to a freezing temperature of about −40° C., with a freezing ramp rate ranging from about 0.1° C./min to 1.0° C./min, and holding the chamber at the freezing temperature for one hour or more (e.g., one hour, etc.).
subjecting, after the initial freezing step, the composition to a primary drying step comprising raising the temperature of the lyophilization chamber to a primary drying temperature ranging from −25° C. to −35° C. with a ramp rate ranging from 0.1° C./min up to 1.0° C./min, wherein the primary drying temperature is about −29° C. or −30° C.; and maintaining the chamber at the primary drying temperature for 25 or more hours at a pressure of about 57-60 mTorr; and
subjecting, after the primary drying step, the composition to a secondary drying step comprising raising the temperature of the lyophilization chamber to a secondary drying temperature ranging from 0° C. to 40° C. with a ramp rate ranging from 0.1° C./min up to 1.0° C./min, wherein the chamber is held at a pressure of about 57-60 mTorr and wherein the secondary drying temperature and cycle time is selected from the group consisting of:
about 5° C. for about 48 hours;
about 15° C. for about 12 hours; or
about 25° C. for about 6 hours.

This lyophilization process may be used for any of the compositions provided herein.

### Administration

The invention involves administration of a composition comprising an RNA-LNP molecule, wherein the composition has been lyophilized and reconstituted, to a delivery site in a vertebrate. The delivery site will usually be muscle tissue, such as skeletal muscle. Alternatives to intramuscular administration include, but are not limited to: intradermal, intranasal, intraocular, subcutaneous, intraperitoneal, intravenous, interstitial, buccal, transdermal, or sublingual administration. Intradermal and intramuscular administration are two preferred routes.

Administration can be achieved in various ways. For instance, injection via a needle (e.g. a hypodermic needle) can be used, particularly for intramuscular, subcutaneous, intraocular, intraperitoneal or intravenous administration. Needle-free injection can be used as an alternative.

Intramuscular injection is the preferred way of administering RNA according to the invention. Injection into the upper arm, deltoid or thigh muscle (e.g. anterolateral thigh) is typical.

The delivery site includes non-immune cells, such as muscle cells (which may be multinucleated and may be arranged into fascicles) and/or fibroblasts. RNA enters the cytoplasm of these cells after (or while) being administered to the delivery site. Entry can be via endocytosis e.g. across the sarcolemma of a muscle cell, or across the cell membrane of a fibroblast. After RNA entry (and escape from the endosome), it can bind to RNA helicases such as RIG-I(RLR-1), MDA5 (RLR-2) and/or LGP2 (RLR-3). This binding can initiate RLR-mediated signalling, thereby triggering innate immune pathways which enhance the immunogenic effect of the delivered RNA. Even if the delivered RNA is single-stranded, it can form double-stranded RNA either during replication or due to its secondary structure, which means that the RNA can also initiate PKR-mediated signalling, again leading to the triggering of innate immune pathways. Both RLR-mediated and PKR-mediated signalling can lead to secretion of type I interferons (e.g. interferon a and/or B) by the non-immune cells. The non-immune cells may undergo apoptosis after transfection.

The delivery site also includes immune cells, such as macrophages (e.g. bone marrow derived macrophages), dendritic cells (e.g. bone marrow derived plasmacytoid dendritic cells and/or bone marrow derived myeloid dendritic cells), monocytes (e.g. human peripheral blood monocytes), etc. These immune cells can be present at the delivery site at the time of administration, but will usually infiltrate the delivery site after administration. For example, the tissue damage caused by invasive administration (e.g. caused at the delivery site by a needle) can cause immune cells to infiltrate the damaged area. These infiltrating cells will encounter the RNA which is now at the delivery site and RNA can enter the cytoplasm of these immune cells e.g. via endocytosis. After RNA entry the response of these immune cells includes secretion of type I interferons and/or pro-inflammatory cytokines. The RNA can cause this effect via pattern-recognition receptors, such as toll-like receptors (e.g. TLR7), intracellular helicases (e.g. RIG-I), and PKR (dsRNA-dependent protein kinase). The RNA may or may not be translated by the immune cells, and so the immune cells may or may not express the immunogen. If the immunogen is expressed by the immune cell then it may be presented by the immune cell's MHC-I and/or MHC-II. If the immunogen is not expressed by the immune cell then it may instead be captured by the immune cell from other cells (e.g. non-immune cells) which had taken up RNA and expressed the immunogen, and the immunogen can thus be presented by the immune cell's MHC-II and/or MHC-I. Antigen presentation will generally occur in draining lymph nodes after immune cells have migrated away from the delivery site.

The infiltration of immune cells at a delivery site can be observed directly e.g. by using labelled immune cells and then either in vivo imaging techniques or biopsy sampling. The ability of RNA to enter the immune cells and the non-immune cells can be detected by sequence-specific detection techniques performed in situ or on an excised sample.

### Subjects

The present invention is generally intended for mammalian subjects, in particular human subjects. The subject may be a wild or domesticated animal. Mammalian subjects include for example cats, dogs, pigs, sheep, horses or cattle. In one embodiment the invention, the subject is human.

The subject to be treated using the method of the invention may be of any age.

In one embodiment the subject is a human infant (up to 12 months of age). In another embodiment the subject is a human child between the ages of 6 months up to 11 years, 5 years up to 11 years, 12 years up to 16 or 17 years. In one embodiment the subject is a human child (less than 18 years of age). In one embodiment the subject is an adult human (aged 18-59). In one embodiment the subject is an older human (aged 60 or greater).

Doses administered to younger children, such as less than 12 years of age, may be reduced relative to an equivalent adult dose.

The methods of the invention are suitably intended for prophylaxis of infectious diseases, i.e. for administration to a subject which is not infected with a pathogen. In other embodiments the methods of the invention may be intended for treatment, e.g. for the treatment of infectious diseases, i.e. for administration to a subject which is infected with a pathogen.

Compositions of the invention may be co-administered with other formulations such as adjuvants. Consequently, it will be appreciated that a range of formulation possibilities exist. A reasonable balance is desirable between practical considerations such as: manufacture, storage and distribution of the mRNA. The carrier-formulated mRNA and adjuvant are desirably administered to locations with sufficient spatial proximity such that the adjuvant effect is adequately maintained. For example, spatial proximity is sufficient to maintain at least 50%, especially at least 75% and in particular at least 90% of the adjuvant effect seen with administration to the same location. The adjuvant effect seen with administration to the same location is defined as the level of increase observed as a result of administration of carrier-formulated mRNA and squalene emulsion adjuvant to the same location compared with administration of carrier-formulated mRNA alone. The carrier-formulated mRNA and adjuvant are desirably administered to a location draining to the same lymph node, such as to the same limb, in particular to the same muscle.

### Adjuvant

Suitably carrier-formulated reconstituted mRNA and adjuvant are administered intramuscularly to the same muscle. In certain embodiments, the carrier-formulated mRNA and adjuvant are administered to the same location.

The spatial separation of administration locations may be at least 5 mm, such as at least 1 cm. The spatial separation of administration locations may be less than 10 cm, such as less than 5 cm apart.

When administered as separate formulations, the carrier-formulated mRNA and adjuvant are desirably administered with sufficient temporal proximity such that the adjuvant effect is adequately maintained. For example, temporal proximity is sufficient to maintain at least 50%, especially at least 75% and in particular at least 90% of the adjuvant effect seen with administration at the same time. The adjuvant effect seen with administration at the same time is defined as the level of increase observed as a result of administration of carrier-formulated mRNA and adjuvant at (essentially) the same time compared with administration of carrier-formulated mRNA without adjuvant.

When administered as separate formulations, carrier-formulated mRNA and adjuvant may be administered within 12 hours. Suitably the carrier-formulated mRNA and adjuvant are administered within 6 hours, especially within 2 hours, in particular within 1 hour, such as within 30 minutes and especially within 15 minutes (e.g. within 5 minutes).

When administered as separate formulations, carrier-formulated mRNA and adjuvant may be administered within 84 hours, such as within 60 hours, especially within 36 hours, in particular within 24 hours. In one embodiment the carrier-formulated mRNA and adjuvant are administered within 12 to 36 hours. In another embodiment the carrier-formulated mRNA and adjuvant are administered within 36 to 84 hours.

The delay between administration of the carrier-formulated mRNA and adjuvant may be at least 5 seconds, such as 10 seconds, and in particular at least 30 seconds.

When administered as separate formulations, if the carrier-formulated mRNA and adjuvant are administered with a delay, the carrier-formulated mRNA may be administered first and the adjuvant administered second. Alternatively, the adjuvant is administered first and the carrier-formulated mRNA is administered second. Appropriate temporal proximity may depend on the order of administration.

In addition to co-formulated or separately formulated presentations of carrier-formulated mRNA and adjuvant for direct administration, the carrier-formulated mRNA and adjuvant may initially be provided in various forms which facilitate manufacture, storage and distribution. For example, certain components may have limited stability in liquid form, certain components may not be amendable to drying, certain components may be incompatible when mixed (either on a short- or long-term basis). Independent of whether carrier-formulated mRNA and are co-formulated at administration, they may be provided in separate containers the contents of at least some of which are subsequently combined. The skilled person will appreciate that many possibilities exist, although it is generally desirable to have a limited number of containers and limited number of required steps to prepare the final co-formulation or separate formulations for administration.

Carrier-formulated mRNA (e.g., liposomal encapsulated mRNA) may be provided in liquid or dry (e.g. lyophilized) form. The preferred form will depend on factors such as the precise nature of the carrier-formulated mRNA, e.g. if the carrier-formulated mRNA is amenable to drying, or other components which may be present.

The adjuvant may be provided in liquid or dry form. The preferred form will depend on the precise nature of adjuvant, e.g. if capable of self-emulsification, and any other components present.

The invention provides a composition comprising carrier-formulated mRNA encoding an antigen and an adjuvant. Typically carrier-formulated mRNA encoding an antigen and adjuvant are provided as a liquid co-formulation. A liquid co-formulation enables convenient administration at the point of use.

In other embodiments the carrier-formulated mRNA encoding an antigen and adjuvant are provided as a dry co-formulation, the dry co-formulation being reconstituted prior to administration. A dry co-formulation, where the components of the formulation are amendable to such presentation, may improve stability and thereby facilitate longer storage.

The carrier-formulated mRNA encoding an antigen and adjuvant may be provided in separate containers. The invention therefore provides carrier-formulated mRNA encoding an antigen for use with an adjuvant. Also provided is an adjuvant for use with carrier-formulated mRNA encoding an antigen. Further provided is a kit comprising:
(i) a first container comprising carrier-formulated mRNA encoding an antigen; and
(ii) a second container comprising an adjuvant.

The carrier-formulated mRNA encoding an antigen may be in liquid form and the adjuvant may be in liquid form. In such cases the contents of the first and second containers may be intended for combination to provide a co-formulation for administration. Alternatively, the contents of each container may be intended for separate administration as the first and second formulations.

The carrier-formulated mRNA encoding an antigen may be in dry form and the adjuvant may be in liquid form. In such cases the contents of the first and second containers may be intended for combination to provide a co-formulation for administration. Alternatively, the carrier-formulated mRNA encoding an antigen may be intended to be reconstituted prior to the contents of each container being used for separate administration as the first and second formulations.

The adjuvant may be in dry form and the carrier-formulated mRNA encoding an antigen may be in liquid form. In such cases the contents of the first and second containers may be intended for combination to provide a co-formulation for administration. Alternatively, the adjuvant may be intended to be reconstituted prior to the contents of each container being used for separate administration as the first and second formulations.

The carrier-formulated mRNA may be in dry form (lyophilized) and the adjuvant may be in dry form. In such cases the contents of the first and second containers may be intended for reconstitution and combination to provide a co-formulation for administration. Reconstitution may occur separately before combination, or the contents of one container may be reconstituted and then used to reconstitute the contents of the other container. Alternatively, the contents of the first and second containers may be intended for reconstitution prior to the contents of each container being used for separate administration as the first and second formulations. In other cases, the carrier-formulated mRNA may be in dry form, and may be reconstituted with the adjuvant in liquid form.

The precise composition of liquid used for reconstitution will depend on both the contents of a container being reconstituted and the subsequent use of the reconstituted contents e.g. if they are intended for administration directly or may be combined with other components prior to administration. A composition (such as those containing carrier-formulated mRNA encoding an antigen or adjuvant) intended for combination with other compositions prior to administration need not itself have a physiologically acceptable pH or a physiologically acceptable tonicity; a formulation intended for administration should have a physiologically acceptable pH and should have a physiologically acceptable osmolality.

The pH of a liquid preparation is adjusted in view of the components of the composition and necessary suitability for administration to the human subject. The pH of a formulation is generally at least 4, especially at least 5, in particular at least 5.5 such as at least 6. The pH of a formulation is generally 9 or less, especially 8.5 or less, in particular 8 or less, such as 7.5 or less. The pH of a formulation may be 4 to 9, especially 5 to 8.5, in particular 5.5 to 8, such as 6.5 to 7.4 (e.g. 6.5 to 7.1).

For parenteral administration, solutions should have a physiologically acceptable osmolality to avoid excessive cell distortion or lysis. A physiologically acceptable osmolality will generally mean that solutions will have an osmolality which is approximately isotonic or mildly hypertonic. Suitably the formulations for administration will have an osmolality of 250 to 750 mOsm/kg, especially 250 to 550 mOsm/kg, in particular 270 to 500 mOsm/kg, such as 270 to 400 mOsm/kg. Osmolality may be measured according to techniques known in the art, such as by the use of a commercially available osmometer, for example the Advanced® Model 2020 available from Advanced Instruments Inc. (USA).

Liquids used for reconstitution will typically be substantially aqueous, such as water for injection, phosphate buffered saline and the like. As mentioned above, the requirement for buffer and/or tonicity modifying agents will depend on both the contents of the container being reconstituted and the subsequent use of the reconstituted contents. Buffers may be selected from acetate, citrate, histidine, maleate, phosphate, succinate, tartrate and TRIS. The buffer may be a phosphate buffer such as Na/Na2PO4, Na/K2PO4 or K/K2PO4.

SAM-LNPs are formulated based on RNA concentration, including theoretical concentrations pre- and post-lyo. For example, a lyo vial is filled with 700 μL of 1 μg/mL SAM-LNP to produce a vial theoretically containing 0.7 ug of RNA. Post lyo, the cake is reconstituted in 646 μL of water for injection (WFI), to account for volume expansion of the solids during lyophilization. Upon reconstitution, the concentration of SAM-LNP is expected to be the same as pre-lyo (as may be confirmed with testing using Ribogreen). A dose of 0.5 mL (500 uL-human dose) is expected to contain 0.5 ug of SAM-LNP. Vials may contain multiple doses, by scaling the amount of SAM-LNP included in a vial.

Suitably, the formulations used in the present invention have a dose volume of between 0.05 ml and 1 ml, such as between 0.1 and 0.6 ml, in particular a dose volume of 0.45 to 0.55 ml, such as 0.5 ml. The volumes of the compositions used may depend on the subject, delivery route and location, with smaller doses being given by the intradermal route or if both the carrier-formulated mRNA and adjuvant are delivered to the same location. A typical human dose for administration through routes such as intramuscular, is in the region of 200 ul to 750 ml, such as 400 to 600 ul, in particular about 500 ul, such as 500 ul.

If two liquids are intended to be combined, for example for co-formulation if the carrier-formulated mRNA is in liquid form and the adjuvant is in liquid form, the volume of each liquid may be the same or different. Volumes for combination will typically be in the range of 10:1 to 1:10, such as 2:1 to 1:2. Suitably the volume of each liquid will be substantially the same, such as the same. For example a 250 ul volume of carrier-formulated mRNA in liquid form may be combined with a 250 ul volume adjuvant in liquid form to provide a co-formulation dose with a 500 ul volume, each of the carrier-formulated mRNA and adjuvant being diluted 2-fold during the combination.

It is common where liquids are to be transferred between containers, such as from a vial to a syringe, to provide ‘an overage’ which ensures that the full volume required can be conveniently transferred. The level of overage required will depend on the circumstances but excessive overage should be avoided to reduce wastage and insufficient overage may cause practical difficulties. Overages may be of the order of 20 to 100 ul per dose, such as 30 ul or 50 ul. For example, a typical 10 dose container of doubly concentrated squalene emulsion adjuvant (250 ul per dose) may contain around 2.85 to 3.25 ml of squalene emulsion adjuvant.

Carrier-formulated mRNA and adjuvant in liquid form may be provided in the form of a multichamber syringe. The use of multi-chamber syringes provides a convenient method for the separate sequential administration of the carrier-formulated mRNA and adjuvant. Multi-chamber syringes may be configured to provide concurrent but separate delivery of the carrier-formulated mRNA and adjuvant, or they may be configured to provide sequential delivery (in either order).

In other configurations of multichambered syringes, the carrier-formulated mRNA may be provided in dry form (e.g., freeze-dried) in one chamber and reconstituted by the adjuvant contained in the other chamber before administration.

Examples of multi-chamber syringes may be found in disclosures such as WO2016/172396, although a range of other configurations are possible.

Formulations are preferably sterile.

Approaches for establishing strong and lasting immunity often include repeated immunisation, i.e. boosting an immune response by administration of one or more further doses. Such further administrations may be performed with the same immunogenic compositions (homologous boosting) or with different immunogenic compositions (heterologous boosting). The present invention may be applied as part of a homologous or heterologous prime/boost regimen, as either the priming or a/the boosting immunisation.

Administration of the carrier-formulated mRNA and adjuvant may therefore be part of a multi-dose administration regime. For example, the carrier-formulated mRNA and adjuvant may be provided as a priming dose in a multidose regime, especially a two- or three-dose regime, in particular a two-dose regime. The carrier-formulated mRNA and adjuvant may be provided as a boosting dose in a multidose regime, especially a two- or three-dose regime, such as a two-dose regime.

Priming and boosting doses may be homologous or heterologous. Consequently, the carrier-formulated mRNA and adjuvant may be provided as a priming dose and boosting dose(s) in a homologous multidose regime, especially a two- or three-dose regime, in particular a two-dose regime. Alternatively, the carrier-formulated mRNA and adjuvant may be provided as a priming dose or boosting dose in a heterologous multidose regime, especially a two- or three-dose regime, in particular a two-dose regime, and the boosting dose(s) may be different (e.g. a different carrier-formulated mRNA; or an alternative antigen presentation such as protein or virally vectored antigen—with or without adjuvant).

The time between doses may be two weeks to six months, such as three weeks to three months. In aspects, the time between doses is at least 21 days, 28 days, 42 days, 45 days, or 60 days. In aspects, the time between doses is at least 8 weeks, 10 weeks, 12 weeks, 14 weeks, or 16 weeks. Periodic longer-term booster doses may also be provided, such as every 2 to 10 years.

The adjuvant may be administered to a subject separately from carrier-formulated mRNA, or the adjuvant may be combined, either during manufacturing or extemporaneously, with carrier-formulated mRNA to provide an immunogenic composition for combined administration.

### Methods of Treatment and Medical Uses

RNA delivery according to the invention is for eliciting an immune response in vivo against an immunogen of interest. The immune response is preferably protective and preferably involves antibodies and/or cell-mediated immunity. The method may raise a booster response.

By raising an immune response the vertebrate can be protected against various diseases and/or infections e.g. against bacterial and/or viral diseases as discussed above. RNA-containing compositions are immunogenic, and are more preferably vaccine compositions. Vaccines according to the invention may either be prophylactic (i.e. to prevent infection) or therapeutic (i.e. to treat infection), but will typically be prophylactic.

The vertebrate is preferably a mammal, such as a human or a large veterinary mammal (e.g. horses, cattle, deer, goats, pigs). Where the vaccine is for prophylactic use, the human is preferably a child (e.g. a toddler or infant) or a teenager; where the vaccine is for therapeutic use, the human is preferably a teenager or an adult. A vaccine intended for children may also be administered to adults e.g. to assess safety, dosage, immunogenicity, etc.

Vaccines prepared according to the invention may be used to treat both children and adults. Thus a human patient may be less than 1 year old, less than 5 years old, 1-5 years old, 5-15 years old, 15-55 years old, or at least 55 years old. Preferred patients for receiving the vaccines are the elderly (e.g. >50 years old, >60 years old, and preferably >65 years), the young (e.g. <5 years old), hospitalised patients, healthcare workers, armed service and military personnel, pregnant women, the chronically ill, or immunodeficient patients. The vaccines are not suitable solely for these groups, however, and may be used more generally in a population.

Compositions of the invention will generally be administered directly to a patient. Direct delivery may be accomplished by parenteral injection (e.g. subcutaneously, intraperitoneally, intravenously, intramuscularly, or to the interstitial space of a tissue; unlike WO02/02606, intraglossal injection is not typically used with the present invention), or mucosally, such as by rectal, oral (e.g. tablet, spray), vaginal, topical, transdermal or transcutaneous, intranasal, ocular, aural, pulmonary or other mucosal administration. Injection may be via a needle (e.g. a hypodermic needle), but needle-free injection may alternatively be used. A typical intramuscular dose is 0.5 ml.

The invention may be used to elicit systemic and/or mucosal immunity, preferably to elicit an enhanced systemic and/or mucosal immunity.

Dosage can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Multiple doses will typically be administered at least 1 week apart (e.g. about 2 weeks, about 3 weeks, about 4 weeks, about 6 weeks, about 8 weeks, about 10 weeks, about 12 weeks, about 16 weeks, etc.). In one embodiment, multiple doses may be administered approximately 6 weeks, 10 weeks and 14 weeks after birth, e.g. at an age of 6 weeks, 10 weeks and 14 weeks, as often used in the World Health Organisation's Expanded Program on Immunisation (“EPI”). In an alternative embodiment, two primary doses are administered about two months apart, e.g. about 7, 8 or 9 weeks apart, followed by one or more booster doses about 6 months to 1 year after the second primary dose, e.g. about 6, 8, 10 or 12 months after the second primary dose. In a further embodiment, three primary doses are administered about two months apart, e.g. about 7, 8 or 9 weeks apart, followed by one or more booster doses about 6 months to 1 year after the third primary dose, e.g. about 6, 8, 10, or 12 months after the third primary dose.

### General Embodiments

In some embodiments of the invention, the RNA includes no modified nucleotides (see above). In other embodiments the RNA can optionally include at least one modified nucleotide.

The practice of the present invention will employ, unless otherwise indicated, conventional methods of chemistry, biochemistry, molecular biology, immunology and pharmacology, within the skill of the art. Such techniques are explained fully in the literature. See, e.g., Methods In Enzymology (S. Colowick and N. Kaplan, eds., Academic Press, Inc.); Handbook of Experimental Immunology, Vols. I-IV (D. M. Weir and C. C. Blackwell, eds, 1986, Blackwell Scientific Publications); Sambrook et al. (2001) Molecular Cloning: A Laboratory Manual, 3rd edition (Cold Spring Harbor Laboratory Press); Handbook of Surface and Colloidal Chemistry (Birdi, K. S, ed., CRC Press, 1997); Ausubel et al. (eds) (2002) Short protocols in molecular biology, 5th edition (Current Protocols); Molecular Biology Techniques: An Intensive Laboratory Course, (Ream et al., eds., 1998, Academic Press); and PCR (Introduction to Biotechniques Series), 2nd ed. (Newton & Graham eds., 1997, Springer Verlag), etc.

References to charge, to cations, to anions, to zwitterions, etc., are taken at pH 7.

TLR3 is the Toll-like receptor 3. It is a single membrane-spanning receptor which plays a key role in the innate immune system. Known TLR3 agonists include poly(I: C). “TLR3” is the approved HGNC name for the gene encoding this receptor, and its unique HGNC ID is HGNC: 11849. The RefSeq sequence for the human TLR3 gene is GI: 2459625.

TLR7 is the Toll-like receptor 7. It is a single membrane-spanning receptor which plays a key role in the innate immune system. Known TLR7 agonists include e.g. imiquimod. “TLR7” is the approved HGNC name for the gene encoding this receptor, and its unique HGNC ID is HGNC: 15631. The RefSeq sequence for the human TLR7 gene is GI: 67944638.

TLR8 is the Toll-like receptor 8. It is a single membrane-spanning receptor which plays a key role in the innate immune system. Known TLR8 agonists include e.g. resiquimod. “TLR8” is the approved HGNC name for the gene encoding this receptor, and its unique HGNC ID is HGNC: 15632. The RefSeq sequence for the human TLR8 gene is GI: 20302165.

The RIG-I-like receptor (“RLR”) family includes various RNA helicases which play key roles in the innate immune system (see Yoneyama & Fujita (2007) Cytokine & Growth Factor Reviews 18:545-51). RLR-1 (also known as RIG-I or retinoic acid inducible gene I) has two caspase recruitment domains near its N-terminus. The approved HGNC name for the gene encoding the RLR-1 helicase is “DDX58” (for DEAD (Asp-Glu-Ala-Asp) box polypeptide 58) and the unique HGNC ID is HGNC: 19102. The RefSeq sequence for the human RLR-1 gene is GI: 77732514. RLR-2 (also known as MDA5 or melanoma differentiation-associated gene 5) also has two caspase recruitment domains near its N-terminus. The approved HGNC name for the gene encoding the RLR-2 helicase is “IFIH1” (for interferon induced with helicase C domain 1) and the unique HGNC ID is HGNC: 18873. The RefSeq sequence for the human RLR-2 gene is GI; 27886567. RLR-3 (also known as LGP2 or laboratory of genetics and physiology 2) has no caspase recruitment domains. The approved HGNC name for the gene encoding the RLR-3 helicase is “DHX58” (for DEXH (Asp-Glu-X-His) box polypeptide 58) and the unique HGNC ID is HGNC: 29517. The RefSeq sequence for the human RLR-3 gene is GI: 149408121.

PKR is a double-stranded RNA-dependent protein kinase. It plays a key role in the innate immune system. “EIF2AK2” (for eukaryotic translation initiation factor 2-alpha kinase 2) is the approved HGNC name for the gene encoding this enzyme, and its unique HGNC ID is HGNC: 9437. The RefSeq sequence for the human PKR gene is GI: 208431825.

### Making the Lyophilized Composition

The lyophilized composition can be made by any technique provided herein that produces a desired amount of SAM-LNPs or mRNA-LNPs. In addition, it is desirable that most of the mRNA is contained in LNPs, and preferably all or substantially all of the mRNA is contained in LNPs, as discussed above. It is convenient to initially make an aqueous buffered solution containing SAM-LNPs and to add concentrations of stock solutions containing excipients to the aqueous buffered solution. This aqueous buffered solution with excipients is then lyophilized by the techniques described herein until water has been substantially or completely removed from the composition. It is understood that small amounts of water may remain in the composition, either in the core (inside) of the LNPs and/or on the exterior of the LNPs. The lyophilized composition is then stored at a convenient temperature and for a time necessary for distribution and administration to patients.

### Making RNA Containing Lipid Nanoparticles

The SAM-LNPs can be made by variety of different techniques known in the art. A specific technique is described herein (e.g., in the Examples below). These SAM-LNPs can be stored in either liquid, frozen or lyophilized form.

### The Pre-Lyophilized Composition

The composition to be lyophilized (containing both SAM-LNPs and excipients) may be an aqueous buffered composition that is suitable for injection, but that is intended to be lyophilized to provide a stable formulation that can be stored and transported at the temperatures described herein. In this embodiment, the pre-lyophilized composition will have a chemical composition and physical properties that closely match the composition and properties of the reconstituted composition, as described below.

The amount of encapsulated mRNA is desirably about the same before lyophilization, optionally after lyophilization, and after reconstitution. The percentage of mRNA that is ideally encapsulated is described in other parts of this application, and is typically is greater than 95%, 96%, 97%, 98%, 99% or more. The mRNA is present in an effective amount upon reconstitution.

### The Lyophilized Composition

The relative amount of the mRNA-LNPs are typically the same as the relative amounts in the pre-lyophilized composition as described immediately above. The lyophilized composition will contain the mRNA-LNPs as well as any other non-liquid components of the liquid or aqueous composition that are not removed during lyophilization. The lyophilized composition will be in the form of a loose dry powder or a dry cake or a combination of the two. The dry powder or cake will have a certain volume that may be increased, may remain the same or may be decreased during reconstitution after enough liquid for reconstitution is added. Ideally, the dry powder or dry cake or combination of the two will be easily dispersed in the water for reconstitution with the aid of agitation of a degree typical in the pharmaceutical arts for reconstituting lyophilized vaccines at the facility where injections are administered prior to injection.

### Properties of Pre- and Post-Lyophilized Compositions

In certain embodiments, the encapsulation of the liquid pre-lyophilization control (before lyophilization) is within about 20%, about 15%, about 10%, about 5%, about 4%, about 3% or more of the reconstituted lyophilized pharmaceutical composition. The liquid pre-lyophilization control comprises about 60-250 μg/mL SAM-LNP, 20 mM Tris, 5 mM NaCl, 7.5% sucrose, at pH 8.0.

In certain embodiments, in vitro potency or efficacy of the pharmaceutical composition before lyophilization (e.g., liquid pre-lyophilization control) and after lyophilization (e.g., reconstituted lyophilized pharmaceutical composition) are similar. The liquid pre-lyophilization control comprises about 60-250 μg/mL SAM-LNP, 20 mM Tris 5 mM NaCl, 7.5% sucrose, at pH 8.0.

### Reconstituting the Lyophilized Composition

The vaccine can be part of a kit that contains the lyophilized composition of the present invention in one container and a liquid to be used for reconstitution in another container. The liquid for reconstitution can be sterile water for injection if the lyophilized composition contains all the necessary buffer components, adjuvants and/or excipients in dry form. In such a situation, after reconstitution with sterile water for injection, the resulting aqueous formulation will have acceptable properties for injection.

In one embodiment, an aqueous solution such as sterile water for injection is introduced into the container (such as a sterile vial) housing the lyophilized composition in an amount sufficient to provide the desired concentration of the various components of the vaccine in the injectable solution. The container will typically have enough of the lyophilized composition for multiple doses, such as 2-10 doses or 3-8 doses or 4-8 doses or 5-6 doses. In this situation, the container will contain at least enough vaccine composition for the corresponding number of doses, and usually some extra volume. After the aqueous solution is introduced into the vial, the vial is shaken and inspected for expected and desired visual properties, such as degree of transparency, color, etc. The injectable solution is then ready for being withdrawn from the vial by techniques known in the art, such as by inserting a syringe needle into the vial and withdrawing an appropriate amount of injectable solution into the syringe. Although sterile water for injection may be a suitable liquid for reconstitution, other aqueous solutions such as buffers containing other additive, excipients and/or adjuvants may be included in the liquid for reconstitution if these other additives, excipients and/or adjuvants are not present, or are not present in sufficient quantities, in the lyophilized composition.

Reconstitution can take place at any suitable temperature but will typically take place at room temperature.

When the vaccine is provided as a kit, the kit will typically include 2 or more containers containing different components that need to be mixed prior to injection into the subject being vaccinated. The two or more containers will include, for example, one container that contains the lyophilized composition and one container that contains the liquid for reconstitution. Alternatively, the kit may include one container that contains the lyophilized composition and a syringe of suitable size (packaged in a sterile package) and optionally another container that contains the liquid for reconstitution. Instructions for reconstitution at the clinic site and for administration of the reconstituted vaccine composition to the patient will be included in, on or associated with the various components of the kit.

### Definitions and Meaning of Terms

Unless otherwise explained, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (cds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

“About” or “approximately”, when used to modify a numeric value, means a number that is not statistically different from the referenced numeric value and, when the numeric value relates to the amount of a composition component, means a number not more than 10% below or above the numeric value (not more than 10% below or above the endpoint values if the numeric value is a range). As an example, a composition comprising “about 25 ug” of component A means the composition comprises “22.5-27.5 ug” of component A (10% of 25 is 2.5, so 10% below 25 is 22.5 and 10% above 25 is 27.5; resulting in the range 22.5-27.5). As an example, a composition comprising “approximately 25 ug” of component A means the composition comprises “22.5-27.5 ug” of component A. As a further example, a composition comprising “about 25-30 ug” of component A means the composition comprises “22.5-33 ug” of component A (10% below 25 is 22.5 and 10% above 30 is 33). As a further example, a composition comprising “approximately 25-30 ug” of component A means the composition comprises “22.5-33 ug” of component A. The term ‘about’ also includes the specific percentage specified, for example, about 7.5% includes 7.5% and about 5% includes 5%, etc.

“Adjuvant” means an agent that, or composition comprising an agent, that modulates an immune response in a non-specific manner and accelerates, prolongs, and/or enhances the immune response to an antigen. Such an agent may be an “immunostimulant”. An “adjuvant” herein may be a composition that comprises one or more immunostimulants (in particular, an immunostimulating effective amount of one or more immunostimulants (e.g., a saponin)). A “pharmaceutical-grade adjuvant” means an adjuvant suitable for pharmaceutical use (e.g., an adjuvant comprising one or more purified immunostimulant, in particular comprising an immunologically effective amount of a purified immunostimulant). Therefore and for clarity, an adjuvant administered with an antigen produces an accelerated, prolonged, and/or enhanced immune response than the antigen alone does.

The term “and/or” as used in a phrase such as “A and/or B” is intended to include “A and B,” “A or B.” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone). Similarly, the word “or” is intended to include each of the listed elements individually as well as any combination of the elements (i.e., “or” herein encompasses “and”), unless the context clearly indicates otherwise.

“Antigen” means a molecule, structure, compound, or substance (e.g., a polynucleotides (DNA, RNA), polypeptides, protein complexes) that can stimulate an immune response by producing antigen-specific antibodies and/or an antigen-specific T cell response in a subject (e.g., a human subject). Antigens may be live, inactivated, purified, and/or recombinant. For clarity, an adjuvant is not an antigen at least because an adjuvant cannot (alone) induce antigen-specific immune responses. As used herein, an antigen is immunogenic. The term “antigen” includes all related antigenic epitopes. The term “epitope” means that portion of an antigen that determines its immunological specificity and refers to a site on an antigen to which B and/or T cells respond. “Predominant antigenic epitopes” are those epitopes to which a functionally significant host immune response (e.g., an antibody response or a T-cell response) is made. Thus, the predominant antigenic epitopes are those antigenic moieties that, when recognized by the host immune system, result in a protective immune response. The term “T-cell epitope” refers to an epitope that, when bound to an appropriate MHC molecule, is specifically bound by a T cell (via a T cell receptor). A “B-cell epitope” is an epitope that is specifically bound by an antibody (or B cell receptor molecule).

Articles—As used herein, the singular form of articles “a.” “an” and “the” include plural references unless the content clearly dictates otherwise.

Comprising—The term “comprising” encompasses “including” as well as “consisting” e.g. a composition “comprising” X may consist exclusively of X or may include something additional e.g. X+Y. Throughout the specification, including the claims, where the context permits, the term “comprising” and variants thereof such as “comprises” are to be interpreted as including the stated element (e.g., integer) or elements (e.g., integers) without necessarily excluding any other elements (e.g., integers). Thus a composition “comprising” X may consist exclusively of X or may include something additional e.g. X+Y.

An “effective amount” means an amount sufficient to cause the referenced outcome. An “effective amount” can be determined empirically and in a routine manner using known techniques in relation to the stated purpose. An “immunologically effective amount”, with respect to an antigen or immunogenic composition, is a quantity sufficient to elicit a measurable immune response in a subject (e.g., 0.1-100 ug of antigen). With respect to an adjuvant, an “adjuvanting effective amount” or “immunostimulating effective amount” (in the case of an adjuvant that is an immunostimulant) is a quantity sufficient to modulate an immune response (e.g., 0.1-100 ug of adjuvant). To obtain a protective immune response against a pathogen, it can require multiple administrations of an immunogenic composition. So in the context of, for example, a protective immune response, an “immunologically effective amount” encompasses a fractional dose that contributes in combination with previous or subsequent administrations to attaining a protective immune response.

Endpoints-Unless specifically stated otherwise, providing a numeric range (e.g., “25-30”) is inclusive of endpoints (i.e., includes the values 25 and 30). An endpoint of a range may be excluded by reciting “exclusive of lower endpoint” or “exclusive of upper endpoint”. Both endpoints may be excluded by reciting “exclusive of endpoints”.

“Human dose” means a dose which is in a volume suitable for human use (“human dose volume”) such as 0.25-1.5 ml. For example, a composition formulated in a volume of about 0.5 ml; specifically a volume of 0.45-0.55 ml; or more specifically a volume of 0.5 ml.

Lipid Nanoparticles (LNPs) LNPs are nanoparticles formed from biodegradable and nontoxic negatively charged lipids (such as phospholipids) that encapsulate the mRNA molecules (such as SAM molecules) used in the present invention. Liposomes and LNPs are often considered to be different from each other. For example, the term “liposomes” is often used to refer to particles that have a lipid bilayer surrounding a core, which is usually an aqueous core. In contrast, some LNPs are micellar-like structures (do not have distinct lipid bilayers) encapsulating a drug molecule, such as RNA, in possibly small aqueous pockets. But, unless otherwise expressly indicated or unless clearly understood from the context, when the term “LNPs” is used, it also encompasses “liposomes”.

Nanoparticles-Nanoparticles are small particles (generally on the order of 1-1,000 nanometers) that are used as part of a delivery system for SAM molecules. Nanoparticles include, without limitation, LNPs, CNEs and polymer-based nanoparticles. Nanoparticles include at least the nanoparticles described by Zhao et al, Nanoparticle vaccines, Vaccine, 32 (2014) 327-337.

Nucleic acid, polynucleotide, and oligonucleotide-“Nucleic acid.” “polynucleotide,” and “oligonucleotide” are sometimes used interchangeably and can have similar or overlapping meanings. They are inherently composed of a sequence of nucleotides, each nucleotide comprising a phosphate and a nucleoside, a nucleoside comprising a pentose sugar (e.g. deoxyribose and ribose) and a nucleobase (e.g. a purine comprising adenine or guanine and a pyrimidine comprising cytosine, uracil, N1-methyluracil, and thymine). In some embodiments, the nucleoside (i.e. sugar and nucleobase) can be standard nucleosides (i.e. adenosine, deoxyadenosine, guanosine, deoxyguanosine, thymidine (a.k.a. deoxythymidine), uridine, deoxyuridine, cytidine, or deoxycytidine, or methylates thereof (i.e. 5′-methyluridine) or they may be modified nucleosides (i.e. pseudouridine (a.k.a. 5-(B-D-Ribofuranosyl)pyrimidine-2,4 (1H,3H)-dione or 5-[(2S,3R,4S,5R)-3.4-Dihydroxy-5-(hydroxymethyl)oxolan-2-yl]pyrimidine-2.4 (1H,3H)-dione, CAS No. 1445 Jul. 4, PubChem CID 15047), N1-methyluridine, N1-methylpseudouridine (a.k.a. 5-[(2S,3R,4S.5R)-3,4-Dihydroxy-5-(hydroxymethyl)oxolan-2-yl]-1-methylpyrimidine-2,4-dione, CAS No. 13860-38-3, PubChem CID 99543), or deoxyribose-containing or ribose-containing forms thereof). In some embodiments, the modified nucleotides comprise hypoxanthine, inosine, 8-oxo-adenine, 7-substituted derivatives thereof, dihydrouracil, pseudouridine, N1-methylpseudouridine, 2-thiouracil, 4-thiouracil, 5-aminouracil, 5-(C1-C6)-alkyluracil, 5-methyluracil, 5-(C2-C6)-alkenyluracil, 5-(C2-C6)-alkynyluracil, 5-(hydroxymethyl) uracil, 5-chlorouracil, 5-fluorouracil, 5-bromouracil, 5-hydroxy cytosine, 5-(C1-C6)-alkylcytosine, 5-methylcytosine, 5-(C2-C6)-alkenylcytosine, 5-(C2-C6)alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, or hydrogen (abasic residue). A “nucleic acid,” “polynucleotide,” and “oligonucleotide” can be a stand-alone molecule (i.e. an RNA molecule) or they may be “region,” “sequence,” or “segment” therein, and in this regard, the use of “region,” “sequence,” or “segment” is used to distinguish between such and a stand-alone molecule.

Process and Method Steps-Unless specifically stated, a process comprising a step of mixing two or more components does not require any specific order of mixing. Thus components can be mixed in any order. Where there are three components then two components can be combined with each other, and then the combination may be combined with the third component, etc.

“Purified” means removed from its natural environment and substantially free of impurities from that natural environment (such as other chromosomal and extra-chromosomal DNA and RNA, organelles, and proteins (including other proteins, lipids, or polysaccharides which are also secreted into culture medium or result from lysis of host cells). For clarity and as used herein, an antigen within a pharmaceutical, immunogenic, vaccine, or adjuvant composition is a purified antigen (whether or not the word “purified” is recited). It is understood in the field that for an antigen, agent, adjuvant, additive, vector, molecule, compound, or composition in general to be suitable for pharmaceutical or vaccine use (i.e., “pharmaceutically acceptable”), it must be purified (i.e., not crude). It would be further understood that “purified” is a relative term and that absolute (100%) purity is not required for, e.g., pharmaceutical or vaccine use. A molecule may be at a purity of at least 85%, 86%, 87%, 88%, 89%, 90%, 91%, 92%, 93%, 94% or 95% of a composition's total proteinaceous mass (determined by, e.g., gel electrophoresis). Methods of purification are known and include, e.g., various types of chromatography such as High Performance Liquid Chromatography (HPLC), hydrophobic interaction, ion exchange, affinity, chelating, and size exclusion; electrophoresis; density gradient centrifugation; or solvent extraction. “Isolated” means removed from its natural environment and not linked to a recombinant molecule or structure (e.g., not bound to a recombinant antibody or antibody fragment) including not linked to a laboratory tool (e.g., not linked to a chromatography tool such as not bound to an affinity chromatography column). Hence, an “isolated betacoronavirus antigen”, such as an “isolated modified betacoronavirus Spike protein or Spike protein fragment”, is not on the surface of a betacoronavirus-infected cell or within an infectious betacoronavirus virion or bound to a recombinant antibody or recombinant antibody fragment (which occurs in an ELISA assay, for example). It would be understood that an antigen being bound to an antibody or antibody fragment (through epitope recognition, for example) is different than an antigen being operably linked to an antibody or antibody fragment (operable linkage in that case would use recombinant techniques and produces a molecule that does not occur in nature).

“Recombinant” when used to describe a biological molecule or biological structure (e.g., protein, nucleic acid, organism, cell, vesicle, sacculi, or membrane) means the biological molecule or biological structure is artificially produced (e.g., by laboratory methods), synthetic, and/or has a different structure and/or function than the molecule or structure from which it was obtained or than its wild type counterpart. For clarity, a recombinant molecule or recombinant structure that is synthetic may nonetheless function comparably to its wild type counterpart. For clarification, a “recombinant nucleic acid” or “recombinant polynucleotide” means a nucleic acid/polynucleotide that, by virtue of its origin or manipulation (e.g., by laboratory methods): (1) is not associated with all or a portion of the polynucleotide with which it is associated in nature; and/or (2) is linked to a polynucleotide other than that to which it is linked in nature. A “recombinant protein/polypeptide” thereby encompasses a protein/polypeptide produced by expression of a recombinant polynucleotide. For clarification, a “purified protein” (e.g., a protein suitable for pharmaceutical use) is encompassed within the term “recombinant protein” because a purified protein is both artificially produced and has a different function than the crude protein (or extract or culture) from which it was obtained. A biological molecule or biological structure of the present invention may be described as “artificially produced”. “Heterologous” denotes that the two referenced biological molecules or biological structures are not naturally associated with each other (would not contact each other but—for the hand of man) or that the referenced biological molecule/structure is not in its natural environment. For example, when a nucleic acid molecule is operably linked to another polynucleotide that it is not associated with in nature, the nucleic acid molecule may be referred to as “heterologous” (i.e., the nucleic acid molecule is heterologous to at least the polynucleotide). Similarly, when a polypeptide is in contact with or in a complex with another protein that it is not associated with in nature, the polypeptide may be referred to as “heterologous” (i.e., the polypeptide is heterologous to the protein). Further, when a host cell comprises a nucleic acid molecule or polypeptide that it does not naturally comprise, the nucleic acid molecule and polypeptide may be referred to as “heterologous” (i.e., the nucleic acid molecule is heterologous to the host cell and the polypeptide is heterologous to the host cell).

mRNA-LNPs—The term mRNA-LNPs refers to LNPs that contain mRNA therein. In the context of the present invention, the mRNA will encode a desired antigen. The mRNA can be mRNA such as Self-Amplifying mRNA or a non-replicating mRNA.

SAM (Self-Amplifying mRNA or SAM replicon) or SAM Molecule-SAM or SAM Molecule useful in accordance with the present invention contain at least mRNA that encodes an immunogenic or therapeutic polypeptide that elicits a preventative or therapeutic immune response or a therapeutic response against infection with a pathogen. The SAM molecule includes elements that allow the mRNA to self-replicate in vitro in a cell and/or also to replicate in vivo in an animal and/or human. In aspects, the SAM molecule does not encode structural virion proteins.

SAM RNA Vaccine-A SAM RNA vaccine is a product that comprises a SAM Molecule, formulated with a delivery vehicle, that can elicit an immunological response when administered to a patient.

Substantially—The word “substantially” does not exclude “completely” e.g. a composition which is “substantially free” from Y may be completely free from Y. Where necessary, the word “substantially” may be omitted from the definition of the invention.

Units of mass and concentration—As used herein, ng refers to nanograms, ug or ug refers to micrograms, mg refers to milligrams, mL or ml refers to milliliter, and mM refers to millimolar. Similar terms, such as um, are to be construed accordingly.

Various numbered embodiments (clauses) of the present invention are described below, but the invention is not limited to these embodiments.

### Embodiment A

1. A pharmaceutical composition formulated for stable lyophilization of lipid nanoparticle encapsulated RNA, wherein the formulation comprises:
lipid nanoparticle encapsulated RNA (RNA-LNP); and
sucrose.

2. The pharmaceutical composition of embodiment 1, wherein the lipid nanoparticle encapsulated RNA is mRNA.

3. The pharmaceutical composition of embodiment 2, wherein the mRNA encodes an antigen.

4. The pharmaceutical composition of embodiment 3, wherein the mRNA is replicating mRNA or non-replicating RNA.

5. The pharmaceutical composition of embodiment 4, wherein the replicating mRNA is SAM.

6. The pharmaceutical composition of embodiment 5, wherein the SAM does not encode structural virion proteins.

7. The pharmaceutical composition of any one of embodiments 1 to 6, wherein the RNA-LNP is at a concentration of greater than or equal to about 60 μg/mL.

8. The pharmaceutical composition of embodiment 7, wherein the RNA-LNP is at a concentration of about 60 μg/mL-250 μg/mL.

9. The pharmaceutical composition of any of embodiments 1 to 8, wherein the lipid nanoparticle comprises a cationic lipid, a zwitterionic lipid, a cholesterol, and a PEG.

10. The pharmaceutical composition of embodiment 9, wherein the cationic lipid is RV39 and the zwitterionic lipid is DSPC.

11. The pharmaceutical composition of embodiment 9, wherein the cationic lipid is DlinDMA and the zwitterionic lipid is DSPC.

12. The pharmaceutical composition of embodiment 10 or embodiment 11, wherein PEG is present from about 1 to 2% (w/v).

13. The pharmaceutical composition of any of embodiments 1 to 12, wherein the pharmaceutical composition is formulated as a liquid solution and comprises a buffer.

14. The pharmaceutical composition of embodiment 13, wherein the concentration of sucrose is greater than or equal to about 5% (w/v).

15. The pharmaceutical composition of embodiment 14, wherein the concentration of sucrose ranges from about 5% to about 30% (w/v).

16. The pharmaceutical composition of embodiment 15, wherein the concentration of sucrose ranges from about 5% to 20% (w/v).

17. The pharmaceutical composition of embodiment 16, wherein the concentration of sucrose ranges from about 5% to 10% (w/v).

18. The pharmaceutical composition of embodiment 17, wherein the concentration of sucrose is 7.5% (w/v).

19. The pharmaceutical composition of any one of embodiments 13 to 18, wherein the composition further comprises a plasticizer.

20. The pharmaceutical composition of embodiment 19, wherein the plasticizer is sorbitol or glycerol or ethylene glycol.

21. The pharmaceutical composition of embodiment 20, wherein the plasticizer is glycerol.

22. The pharmaceutical composition of any of embodiments 19 to 21, wherein the plasticizer is present in an amount of about 0.1% to 1% (w/v).

23. The pharmaceutical composition of embodiment 22, wherein the plasticizer is present in an amount of about 0.5% (w/v).

24. The pharmaceutical composition of any of embodiments 13 to 23, wherein the buffer is Tris.

25. The pharmaceutical composition of embodiment 24, wherein the concentration of Tris is 20 mM.

26. The pharmaceutical composition of any of embodiments 13 to 25, wherein the composition further comprises 5 mM NaCl.

27. The pharmaceutical composition of any of embodiments 13 to 26, wherein the pH of the solution ranges from 7.5 to 9.0.

28. The pharmaceutical composition of any of embodiments 13 to 27, wherein the pH of the solution is 8.0.

29. The pharmaceutical composition of embodiment 13, wherein the buffer is histidine buffer.

30. The pharmaceutical composition of embodiment 29, wherein the concentration of histidine buffer is about 20-30 mM.

31. The pharmaceutical composition of any of embodiments 29 to 30, wherein the pH of the solution ranges from about 6 to 7.

32. The pharmaceutical composition of any of embodiments 29 to 31, wherein the pH of the solution is about 6.0.

33. The pharmaceutical composition of any of embodiments 29 to 31, wherein the pH of the solution is about 6.5.

34. The pharmaceutical composition of any of embodiments 29 to 33, wherein the concentration of sucrose is greater than or equal to about 5% (w/v).

35. The pharmaceutical composition of any of embodiments 29 to 34, wherein the concentration of sucrose ranges from about 5% to 30% (w/v).

36. The pharmaceutical composition of any of embodiments 29 to 35, wherein the concentration of sucrose ranges from about 5% to 20% (w/v).

37. The pharmaceutical composition of any of embodiments 29 to 36, wherein the concentration of sucrose ranges from about 5% to 10% (w/v).

38. The pharmaceutical composition of any of embodiments 29 to 37, wherein the concentration of sucrose is 7.5% (w/v).

39. The pharmaceutical composition of any of embodiments 29 to 38, wherein the composition further comprises a plasticizer.

40. The pharmaceutical composition of embodiment 39, wherein the plasticizer is sorbitol or glycerol or ethylene glycol.

41. The pharmaceutical composition of embodiment 40, wherein the plasticizer is glycerol.

42. The pharmaceutical composition of any one of embodiments 39 to 41, wherein the plasticizer is present in an amount of about 0.1% to 1.0% (w/v).

43. The pharmaceutical composition of embodiment 42, wherein the plasticizer is present in an amount of about 0.5% (w/v).

44. The pharmaceutical composition of any of embodiments 1 to 13, wherein the composition further comprises an amino acid.

45. The pharmaceutical composition of embodiment 44, wherein the amino acid is selected from the group consisting of: methionine, histidine, and arginine.

46. The pharmaceutical composition of embodiment 45, wherein the amino acid is histidine.

47. The pharmaceutical composition of any of embodiments 44 to 46, wherein the concentration of the amino acid is about 0.1-1.0% (w/v).

48. The pharmaceutical composition of embodiment 47, wherein the concentration of the amino acid is about 0.5% (w/v).

49. The pharmaceutical composition of any of embodiments 44 to 48, wherein the concentration of sucrose is greater than or equal to 5% (w/v).

50. The pharmaceutical composition of embodiment 49, wherein the concentration of sucrose ranges from about 5% to 30% (w/v).

51. The pharmaceutical composition of embodiment 50, wherein the concentration of sucrose ranges from about 5% to 20% (w/v).

52. The pharmaceutical composition of embodiment 51, wherein the concentration of sucrose ranges from about 5% to 10% (w/v).

53. The pharmaceutical composition of embodiment 52, wherein the concentration of sucrose is 7.5% (w/v).

54. The pharmaceutical composition of any of embodiments 44 to 53, wherein the buffer is Tris.

55. The pharmaceutical composition of embodiment 54, wherein the concentration of Tris is about 20 mM.

56. The pharmaceutical composition of any of embodiments 54 to 55, wherein the composition further comprises about 5 mM NaCl.

57. The pharmaceutical composition of any of embodiments 44 to 56, wherein the pH of the solution ranges from about 7.5 to 9.0.

58. The pharmaceutical composition of embodiment 57, wherein the pH of the solution is about 8.0.

59. The pharmaceutical composition of any embodiments 44 to 58, wherein the composition further comprises a plasticizer.

60. The pharmaceutical composition of embodiment 59, wherein the plasticizer is sorbitol or glycerol or ethylene glycol.

61. The pharmaceutical composition of embodiment 60, wherein the plasticizer is glycerol.

62. The pharmaceutical composition of any of embodiments 59 to 61, wherein the plasticizer is present in an amount of about 0.1 to 1% (w/v).

63. The pharmaceutical composition of embodiment 62, wherein the plasticizer is present in an amount of about 0.5% (w/v).

64. The pharmaceutical composition of any of embodiments 1 to 13, wherein the pharmaceutical composition additionally comprises a secondary sugar.

65. The pharmaceutical composition of embodiment 64, wherein the secondary sugar is selected from the group consisting of trehalose, glucose, maltose or melezitose.

66. The pharmaceutical composition of any of embodiments 64 or 65, wherein the concentration of the secondary sugar ranges from about 0.1 to 5% (w/v).

67. The pharmaceutical composition of embodiment 66, wherein the secondary sugar is at a concentration of about 2.5% (w/v).

68. The pharmaceutical composition of any of embodiments 64 to 67, wherein the concentration of sucrose ranges from about 5% to 10% (w/v).

69. The pharmaceutical composition of any of embodiments 64 to 68, wherein the concentration of sucrose is at a concentration of about 5.0% (w/v).

70. The pharmaceutical composition of any of embodiments 64 to 69, wherein the composition further comprises a plasticizer.

71. The pharmaceutical composition of embodiment 70, wherein the plasticizer is sorbitol or glycerol or ethylene glycol.

72. The pharmaceutical composition of embodiment 71, wherein the plasticizer is glycerol.

73. The pharmaceutical composition of any of embodiments 70 to 72, wherein the plasticizer is present in an amount of about 0.1 to 1% (w/v).

74. The pharmaceutical composition of embodiment 73, wherein the plasticizer is present in an amount of about 0.5% (w/v).

75. The pharmaceutical composition of any of embodiments 64 to 74, wherein the buffer is Tris.

76. The pharmaceutical composition of embodiment 75, wherein the concentration of Tris buffer is 20 mM.

77. The pharmaceutical composition of any of embodiments 75 to 76, wherein the composition further comprises 5 mM NaCl.

78. The pharmaceutical composition of any one of embodiments 64 to 74, wherein the buffer is histidine.

79. The pharmaceutical composition of embodiment 78, wherein the concentration of histidine buffer is about 5 mM to 30 mM.

80. A kit comprising the pharmaceutical composition of any preceding embodiment, wherein the pharmaceutical composition is lyophilized.

81. The kit of embodiment 80, further comprising sterile water for resuspension.

82. A vaccine comprising the pharmaceutical composition of any preceding embodiment, wherein the pharmaceutical composition is lyophilized.

83. A method of lyophilization of the pharmaceutical composition of any preceding embodiment comprising:
placing the composition into a lyophilization chamber;
subjecting the composition to an initial freezing step; and
subjecting, after the initial freezing step, the composition to a primary drying step.

84. The method of lyophilization of embodiment 83, wherein subjecting the composition to the initial freezing step comprises decreasing the temperature of the lyophilization chamber to a freezing temperature of about −40° C., with a freezing ramp rate ranging from about 0.1° C./min to 1.0° C./min.

85. The method of lyophilization of embodiment 84, further comprising holding the chamber at the freezing temperature for one hour or more.

86. The method of lyophilization of any of embodiments 83-85, wherein subjecting the composition to the primary drying step comprises raising the temperature of the lyophilization chamber to a primary drying temperature ranging from −25° C. to −35° C. with a ramp rate ranging from 0.1° C./min up to 1.0° C./min.

87. The method of lyophilization of embodiment 86, wherein the ramp rate is 0.5° C./min. 88. The method of lyophilization of embodiment 86, wherein the primary drying temperature is −29° C. or −30° C.

89. The method of lyophilization of any of embodiments 86-88, further comprising holding the chamber at the primary drying temperature for 25 or more hours at a pressure of about 57 or 60 mTorr.

90. The method of lyophilization of embodiment 89, further comprising holding the chamber at the primary drying temperature for about 30 hours or about 27 hours.

91. The method of lyophilization of any of embodiments 83 to 90, additionally comprising subjecting, after the primary drying step, the composition to a secondary drying step.

92. The method of lyophilization of embodiment 91, wherein the initial value of the percentage entrapment initially decreases and then returns toward the initial value during the secondary drying step.

93. The method of lyophilization of embodiment 91, wherein subjecting the composition to the secondary drying step comprises raising the temperature of the lyophilization chamber to a secondary drying temperature ranging from 0° C. to 40° C. with a ramp rate ranging from 0.1° C./min up to 1.0° C./min.

94. The method of lyophilization of embodiment 93, wherein the ramp rate is 0.5° C./min. 95. The method of lyophilization of any of embodiments 91-93, wherein the chamber is held at a pressure of about 57 or 60 mTorr.

96. The method of lyophilization of any of embodiments 91-95, wherein the secondary drying temperature is about 5° C. for 48 hours.

97. The method of lyophilization of any of embodiments 91-95, wherein the secondary drying temperature is about 15° C. for about 12 hours.

98. The method of lyophilization of any of embodiments 91-95, wherein the secondary drying temperature is about 25° C. for 6 hours.

99. The method of lyophilization of any of embodiments 83 to 98, further comprising:
a) a first thermal equilibrium cycle with a ramp rate of about 1° C. per minute to reach a temperature of 5° C. for a duration of about 0.5 hours; and
b) a second thermal equilibrium cycle with a ramp rate of about 1° C. per minute to reach a temperature of −5° C. for a duration of about 0.5 hours.

100. A reconstituted pharmaceutical composition of any of embodiments 80-99, wherein the percent entrapment of the reconstituted pharmaceutical composition is within 20%, within 10%, within 5%, within 4%, within 3%, within 2%, or within 1% of the pharmaceutical composition prior to lyophilization.

101. A reconstituted pharmaceutical composition of any of embodiments 80-99, wherein the size of the reconstituted pharmaceutical composition is within a range of 110-130 nm.

102. A reconstituted pharmaceutical composition of any of embodiments 80-99, wherein the polydispersity index (PDI) of the reconstituted pharmaceutical composition is about 0.1-0.2.

103. A reconstituted pharmaceutical composition of any of embodiments 80-99, wherein the efficacy of the reconstituted pharmaceutical composition is within 10 percent of the efficacy of the pharmaceutical composition prior to lyophilization.

104. The use of the pharmaceutical composition of embodiments 1-79, the kit of embodiments 80-81, the vaccine of embodiment 82, or the reconstituted pharmaceutical composition of embodiments 100-103 in the manufacture of a medicament for treating a subject in need thereof.

105. The use of the pharmaceutical composition of embodiments 1-79, the kit of embodiments 80-81, the vaccine of embodiment 82, or the reconstituted pharmaceutical composition of embodiments 100-103 in the manufacture of a medicament for prophylaxis in a subject in need thereof.

106. A method for eliciting an immune response in a subject in need thereof comprising administering the pharmaceutical composition of embodiments 1-79, the kit of embodiments 80-81, the vaccine of embodiment 82, or the reconstituted pharmaceutical composition of embodiments 100-103 to the subject.

107. The method of any one of embodiments 104-106, wherein the subject is a human subject.

108. The composition of any one of embodiments 1-79, the kit of embodiments 80-81, the vaccine of embodiment 82, or the reconstituted pharmaceutical composition of embodiments 100-103 for use in medicine.

### Embodiment B

1. A pharmaceutical composition formulated for stable lyophilization of lipid nanoparticle encapsulated RNA comprising:
lipid nanoparticle encapsulated RNA (RNA-LNPs) at a concentration ranging from about 60 μg/mL to 250 μg/mL, wherein the lipid nanoparticle comprises a cationic lipid, a zwitterionic lipid, a cholesterol, and a PEG, and
wherein the pharmaceutical composition comprises:
sucrose in an amount of at least 5% (w/v); and
a buffer.

2. The pharmaceutical composition of embodiment 1, wherein said RNA is mRNA and said RNA-LNPs are mRNA-containing lipid nanoparticles (mRNA-LNPs).

3. The pharmaceutical composition of embodiment 1, wherein said RNA is self-amplifying mRNA (SAM) and said RNA-LNPs are SAM-containing lipid nanoparticles (SAM-LNPs).

4. The pharmaceutical composition of any of embodiments 1-3, wherein the cationic lipid is RV39 and the zwitterionic lipid is DSPC.

5. The pharmaceutical composition of any of embodiments 1-3, wherein the cationic lipid is DlinDMA and the zwitterionic lipid is DSPC.

6. The pharmaceutical composition of any of the preceding embodiments, wherein sucrose is present in an amount of 7.5% (w/v), and wherein the buffer is Tris buffer with NaCl.

7. The pharmaceutical composition of embodiment 6, further comprising one or more of: a plasticizer selected from the group consisting of sorbitol or glycerol in an amount of 0.25%-1.0% (w/v), and preferably about 0.5% (w/v); and an amino acid selected from the group consisting of methionine, histidine, and arginine in an amount of 0.25%-1.0% (w/v), and preferably about 0.5% (w/v).

8. The pharmaceutical composition of any of embodiments 1-5, wherein sucrose is present in an amount of 7.5% (w/v), and wherein the buffer is histidine buffer.

9. The pharmaceutical composition of embodiment 8, wherein histidine buffer is 20 to 30 mM histidine buffer and the pH is from 6 to 6.5.

10. The pharmaceutical composition of embodiment 8, additionally comprising one or more of:
a plasticizer selected from the group consisting of sorbitol or glycerol in an amount of 0.25%-1.0% (w/v), and preferably about 0.5% (w/v); and
   an amino acid selected from the group consisting of methionine, histidine, and arginine in an amount of 0.25%-1.0% (w/v), and preferably about 0.5% (w/v).

11. The pharmaceutical composition of embodiment 1, wherein sucrose is present in an amount of 20% (w/v), and wherein the buffer is a histidine buffer or a Tris buffer.

12. The pharmaceutical composition of embodiment 1, wherein the composition additionally comprises:
a secondary sugar selected from the group consisting of trehalose, glucose, stachyose, or maltose present in an amount of about 2.5% (w/v),
wherein sucrose is present in an amount of 5.0% (w/v); and
wherein the buffer comprises a Tris buffer with NaCl or a histidine buffer.

13. The pharmaceutical composition of embodiment 12, additionally comprising one or more of:
a plasticizer selected from the group consisting of sorbitol or glycerol in an amount of 0.25%-1.0% (w/v), and preferably about 0.5% (w/v); and
an amino acid selected from the group consisting of methionine, histidine, and arginine in an amount of 0.25%-1.0% (w/v), and preferably about 0.5% (w/v).

14. A vaccine comprising the pharmaceutical composition of any preceding claim, wherein the pharmaceutical composition is lyophilized.

15. A kit comprising the lyophilized vaccine of embodiment 14, wherein the kit includes:
the lyophilized vaccine of embodiment 14 in a container;
a sterile needle for injecting a vaccine composition; and/or
a second container containing a sterile aqueous solution and/or an adjuvant.

16. A method of lyophilization of the pharmaceutical composition of any of embodiments 1-13 comprising:
placing the composition into a lyophilization chamber;
subjecting the composition to a freezing step that comprises decreasing the temperature of the lyophilization chamber from an initial temperature to a freezing temperature of about-39 or lower, at a controlled freezing ramp rate and holding the chamber at the freezing temperature to convert water to ice.

17. The method of lyophilization of embodiment 16, where the freezing temperature is between-39-° C. to −80° C.

18. The method of lyophilization of embodiment 16 or embodiment 17, where the controlled freezing ramp rate is from about 0.1° C./min to 2.0° C./min.

19. The method of lyophilization of any of embodiment 16-embodiment 18, where the composition is held at the freezing temperature for 30 minutes or more.

20. A method of lyophilization of the pharmaceutical composition of any of embodiments 1-13 comprising:
placing the composition into a lyophilization chamber;
subjecting the composition to an initial freezing step that comprises decreasing the temperature of the lyophilization chamber to a freezing temperature of about-39 or −40° C., with a freezing ramp rate ranging from about 0.1° C./min to 1.0° C./min, and holding the chamber at the freezing temperature for one hour or more.

21. The method of lyophilization of embodiment 16 further comprising:
subjecting, after the initial freezing step, the composition to a primary drying step comprising raising the temperature of the lyophilization chamber to a primary drying temperature ranging from −25° C. to −35° C. with a ramp rate ranging from 0.1° C./min up to 1.0° C./min; and
maintaining the chamber at the primary drying temperature for 25 or more hours at a pressure of about 57-60 mTorr.

22. The method of lyophilization of embodiment 17 further comprising:
subjecting, after the primary drying step, the composition to a secondary drying step comprising raising the temperature of the lyophilization chamber to a secondary drying temperature ranging from 0° C. to 40° C. with a ramp rate ranging from 0.1° C./min up to 1.0° C./min, wherein the chamber is held at a pressure of about 57-60 mTorr.

23. The method of lyophilization of embodiment 18 wherein the secondary drying temperature is about 5° C. for 48 hours.

24. The method of lyophilization of embodiment 18 wherein the secondary drying temperature is about 15° C. for about 12 hours.

25. The method of lyophilization of any of embodiments 16-20, further comprising:
a first thermal equilibrium cycle with a ramp rate of 1° C. per minute to reach a temperature of 5° C. for a duration 0.5 hours; and
a second thermal equilibrium cycle with a ramp rate of 1° C. per minute to reach a temperature of −5° C. for a duration of 0.5 hours.

26. A reconstituted pharmaceutical composition of any of embodiments 14-21, wherein the percent entrapment of the reconstituted pharmaceutical composition is within 20% of the pharmaceutical composition prior to lyophilization.

27. A reconstituted pharmaceutical composition of any of embodiments 14-21, wherein the size of the reconstituted pharmaceutical composition is within a range of 110-130 nm.

28. A reconstituted pharmaceutical composition of any of embodiments 14-21, wherein the polydispersity index (PDI) of the reconstituted pharmaceutical composition is about 0.1-0.2.

29. A reconstituted pharmaceutical composition of any of embodiments 14-21, wherein the efficacy of the reconstituted pharmaceutical composition is within 10 percent of the efficacy of the pharmaceutical composition prior to lyophilization.

30. A method of lyophilization of embodiment 16, comprising:
placing the composition into a lyophilization chamber;
subjecting the composition to a freezing step that comprises decreasing the temperature of the lyophilization chamber to a freezing temperature of below −52.5° C., at a controlled freezing ramp rate and to convert water to ice and form a frozen composition.

31. The method of lyophilization of embodiment 16 comprising:
placing the composition into a lyophilization chamber;
subjecting the composition to a freezing step that comprises decreasing the temperature of the lyophilization chamber to a freezing temperature of below −40° C., at a controlled freezing ramp and to convert water to ice to form a frozen composition; and
raising the temperature of the lyophilization chamber during a primary drying step to a drying temperature of greater than 0° C. at a reduced pressure and at a controlled ramp rate to cause sublimation of ice to a gas.

32. The method of lyophilization of embodiment 31, wherein the primary drying step is conducted for a period of time less than or equal to 30 hours.

33. The pharmaceutical composition of any of embodiments 1-13 or 26-29 for use in inducing an acceptable immune response in a subject, where the pharmaceutical composition is administered by injection.

34. The use of the pharmaceutical composition of any of embodiments 1-13 or 26-29 for the manufacture of a medicament for use in inducing an acceptable immune response in a subject, wherein the medicament is prepared to be administered by injection.

### EXAMPLES

The following procedures may be used to prepare, lyophilize, and reconstitute mRNA-LNPs as well as assess physical properties in the following Examples.

### Overview of Process for Manufacturing Lyophilized (Freeze Dried) Formulations

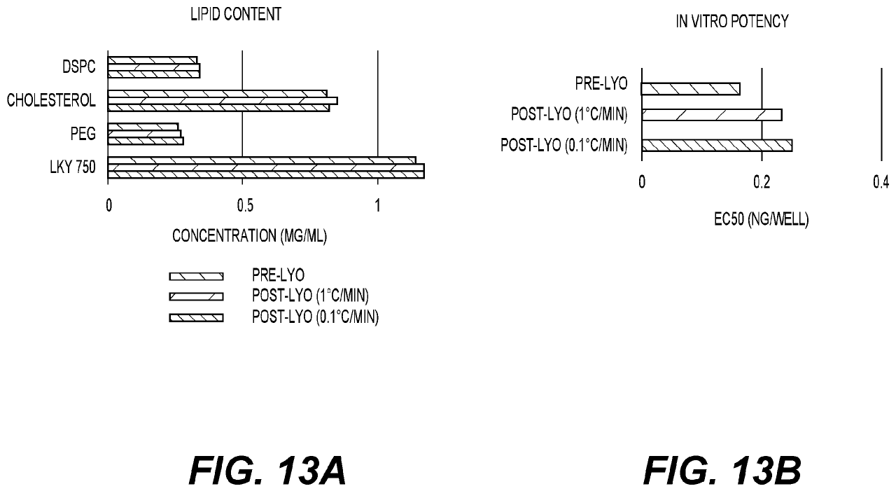

FIG. 12 shows an illustration of a lyophilization process comprising an initial freezing step followed by a primary drying step in which a sublimation process removes unbound water from the composition. A primary drying step is followed by a secondary drying step in which bound water molecules are removed. At the end of secondary drying step, the container is filled with inert gas and stoppered. This flow may be implemented according to the conditions set forth in Table 5 or Tables 6 and 7 with any of the compositions provided herein. Samples may be removed during the lyophilization process by a sample thief. For example, samples may be removed every two hours or at any desired periodic or non-periodic interval during the lyophilization process. The obtained samples may be characterized using the RiboGreen and DLS assays described herein or any other suitable analysis.

The lyophilization process was designed to maximize percent encapsulation of mRNA in LNPs and to preserve the percent encapsulation such that it is comparable to a liquid pre-lyophilization control. Any suitable lyophilization device may be used. For the examples provided herein, a LyoStar3 by SP Scientific was used. Lyophilization steps included loading, thermal equilibrium, freezing, primary drying, and secondary drying.

### Example 1-Formation of LNPs that Encapsulate RNA

RNA-LNPs may be formulated according to the following procedures. Techniques for generating RNA replicons are also summarized as follows. In some aspects, RNA may be in the form of RNA replicons. In other aspects, RNA may be in the form of non-replicating RNA.

Replicons may be based on a hybrid alphavirus genome with non-structural proteins from venezuelan equine encephalitis virus (VEEV), a packaging signal from Sindbis virus, and a 3′ UTR from Sindbis virus or a VEEV mutant. The replicon is about 10 kb long and has a poly-A tail.

Plasmid DNA encoding alphavirus replicons (named: pT7-mVEEV-FL.RSVF or A317; pT7-mVEEV-SEAP or A306; pSP6-VCR-GFP or A50) served as a template for synthesis of RNA in vitro. The replicons contain the alphavirus genetic elements required for RNA replication but lack those encoding gene products necessary for particle assembly; the structural proteins are instead replaced by a protein of interest (either a reporter, such as SEAP or GFP, or an immunogen, such as full-length RSV F protein) and so the replicons are incapable of inducing the generation of infectious particles. A bacteriophage (T7 or SP6) promoter upstream of the alphavirus cDNA facilitates the synthesis of the replicon RNA in vitro and a hepatitis delta virus (HDV) ribozyme immediately downstream of the poly(A)-tail generates the correct 3′-end through its self-cleaving activity.

Following linearization of the plasmid DNA downstream of the HDV ribozyme with a suitable restriction endonuclease, run-off transcripts were synthesized in vitro using T7 or SP6 bacteriophage derived DNA-dependent RNA polymerase. Transcriptions were performed for 2 hours at 37° C. in the presence of 7.5 mM (T7 RNA polymerase) or 5 mM (SP6 RNA polymerase) of each of the nucleoside triphosphates (ATP, CTP. GTP and UTP) following the instructions provided by the manufacturer (Ambion). Following transcription the template DNA was digested with TURBO DNase (Ambion). The replicon RNA was precipitated with LiCI and reconstituted in nuclease-free water. Uncapped RNA was capped post-transcriptionally with Vaccinia Capping Enzyme (VCE) using the ScriptCap m7G Capping System (Epicentre Biotechnologies) as outlined in the user manual; replicons capped in this way are given the “v” prefix e.g. vA317 is the A317 replicon capped by VCE. Post-transcriptionally capped RNA was precipitated with LiCI and reconstituted in nuclease-free water. The concentration of the RNA samples was determined by measuring OD260 nm. Integrity of the in vitro transcripts was confirmed by denaturing agarose gel electrophoresis.

### Liposomal Encapsulation

RNA was encapsulated in liposomes made by the method of El Ouahabi et al. (1996) FEBS Letts 380:108-12 and Maurer et al. (2001) Biophysical Journal, 80:2310-2326. The liposomes were made of 10% DSPC (zwitterionic), 40% DlinDMA (cationic), 48% cholesterol and 2% PEG-conjugated DMG (2 kDa PEG). These proportions refer to the % moles in the total liposome.

This protocol can also be used to encapsulate RNA liposomes made with RV39. Here, liposomes were made of 10% DSPC (zwitterionic), 40% RV39 (cationic), 48% cholesterol and 2% PEG-conjugated DMG (2 kDa PEG). These proportions refer to the % moles in the total liposome.

DlinDMA (1,2-dilinoleyloxy-N,N-dimethyl-3-aminopropane) was synthesized using the procedure of EP2591103. DSPC (1,2-Diastearoyl-sn-glycero-3-phosphocholine) was purchased from Genzyme. Cholesterol was obtained from Sigma-Aldrich. PEG-conjugated DMG (1,2-dimyristoyl-sn-glycero-3-phosphoethanolamine-N-[methoxy (polyethylene glycol), ammonium salt), DOTAP (1,2-dioleoyl-3-trimethylammonium-propane, chloride salt) and DC-chol (3B-[N-(N′.N′-dimethylaminoethane)-carbamoyl]cholesterol hydrochloride) were from Avanti Polar Lipids.

Briefly, lipids were dissolved in ethanol (2 ml), a RNA replicon was dissolved in buffer (2 ml, 100 mM sodium citrate, pH 6) and these were mixed with 2 ml of buffer followed by 1 hour of equilibration. The mixture was diluted with 6 ml buffer then filtered. The resulting product contained liposomes, with ˜95% encapsulation efficiency.

For example, in one particular method, fresh lipid stock solutions were prepared in ethanol. 37 mg of DlinDMA, 11.8 mg of DSPC, 27.8 mg of cholesterol and 8.07 mg of PEG-DMG were weighed and dissolved in 7.55 mL of ethanol. The freshly prepared lipid stock solution was gently rocked at 37° C. for about 15 min to form a homogenous mixture. Then, 755 μL of the stock was added to 1.245 mL ethanol to make a working lipid stock solution of 2 mL. This amount of lipids was used to form liposomes with 250 ug RNA. A 2 mL working solution of RNA was also prepared from a stock solution of ˜1 μg/uL in 100 mM citrate buffer (pH 6). Three 20 mL glass vials (with stir bars) were rinsed with RNase Away solution (Molecular BioProducts) and washed with plenty of MilliQ water before use to decontaminate the vials of RNases. One of the vials was used for the RNA working solution and the others for collecting the lipid and RNA mixes (as described later). The working lipid and RNA solutions were heated at 37° C. for 10 min before being loaded into 3cc luer-lok syringes. 2 mL citrate buffer (pH 6) was loaded in another 3 cc syringe. Syringes containing RNA and the lipids were connected to a T mixer (PEEK™ 500 um ID junction, Idex Health Science) using FEP tubing (fluorinated ethylene-propylene; all FEP tubing used had a 2 mm internal diameter and a 3 mm outer diameter; obtained from Idex Health Science). The outlet from the T mixer was also FEP tubing. The third syringe containing the citrate buffer was connected to a separate piece of tubing. All syringes were then driven at a flow rate of 7 mL/min using a syringe pump. The tube outlets were positioned to collect the mixtures in a 20 mL glass vial (while stirring). The stir bar was taken out and the ethanol/aqueous solution was allowed to equilibrate to room temperature for 1 h. 4 ml of the mixture was loaded into a 5 cc syringe, which was connected to a piece of FEP tubing and in another 5 cc syringe connected to an equal length of FEP tubing, an equal amount of 100 mM citrate buffer (pH 6) was loaded. The two syringes were driven at 7 mL/min flow rate using the syringe pump and the final mixture collected in a 20 mL glass vial (while stirring). Next, the mixture collected from the second mixing step (liposomes) were passed through a Mustang Q membrane (an anion-exchange support that binds and removes anionic molecules, obtained from Pall Corporation). Before using this membrane for the liposomes, 4 mL of 1 M NaOH, 4 mL of 1 M NaCl and 10 mL of 100 mM citrate buffer (pH 6) were successively passed through it. Liposomes were warmed for 10 min at 37° C. before passing through the membrane. Next, liposomes were concentrated to 2 mL and dialyzed against 10-15 volumes of 1×PBS using by tangential flow filtration before recovering the final product. The TFF system and hollow fiber filtration membranes were purchased from Spectrum Labs (Rancho Dominguez) and were used according to the manufacturer's guidelines. Polysulfone hollow fiber filtration membranes with a 100 kD pore size cutoff and 8 cm2 surface area were used. For in vitro and in vivo experiments formulations were diluted to the required RNA concentration with 1×PBS.

Alternatively, liposomes suitable for use herein may be made according to the Examples of WO2018220553.

The liposomes may be concentrated and dialyzed against any suitable buffer (e.g., Histidine, Tris, Citrate, HEPES, etc.).

### Formulation

Stock solutions of various components (see, e.g., Table 2) were generated including: sucrose (20% w/v), secondary sugars (e.g., glucose 20% w/v, trehalose 20% w/v, maltose 20% w/v, and melezitose 20% w/v), amino acids (e.g., arginine 10% w/v, methionine 4% w/v, histidine 4% w/v, lysine 10% w/v, and alanine 10% w/v), and plasticizers (e.g., glycerol 20% w/v and sorbitol at 20% w/v).

TABLE 2
Stock Solution

| | Stock | | Amount | Final |
|---|---|---|---|---|
| | Conc. | Weight | weighed | volume |
| Excipient | (% w/v) | (g) | (g) | (mL) |
| Sucrose | 20 | 10 | 10.02 | 50 |
| Glucose | 20 | 5 | 5.04 | 25 |
| Trehalose | 20 | 5 | 5.03 | 25 |
| Maltose | 20 | 5 | 5.00 | 25 |
| Melezitose | 20 | 1 | 1.00 | 5 |
| Histidine | 4 | 0.4 | 0.40 | 10 |
| Methionine | 4 | 0.4 | 0.41 | 10 |
| Glycerol | 20 | 2 | 2.01 | 10 |
| Sorbitol | 20 | 2 | 2.00 | 10 |

All excipients were filtered using a PES membrane filter. Sugars were filtered using a 50 mL, 0.22 um Millipore filter. Excipients 10 mL or less were filtered using a 0.45 μm syringe filter.

### Buffer Preparation

1) Amounts were weighed on weigh boats as indicated in Table 3 below and transferred to a 1L volumetric flask.

TABLE 3
Buffer

| | Quantities | Amount |
|---|---|---|
| Buffer Components | needed (g) | weighed (g) |
| Vol. of buffer needed | 1 | 1 |
| Tris(hydroxymethyl)aminomethane = | 1.06 | 1.06012 |
| 1.06 *x (g) = | | |
| Tris(hydroxymethyl)aminomethane | 1.78 | 1.7805 |
| hydrochloride = 1.78 * x (g) = | | |
| Sodium Chloride = 0.29 * x (g) = | 0.29 | 0.294 |

2) Sterile water was added to the volumetric flask sufficient to the 1L mark, and the flask was stirred for ˜ 10 mins. The pH was measured to ensure 8.0+0.1. The volume was adjusted to 1 L and filtered using a Millipore 0.22 um PES filter (e.g., 0.45 um PES, L/N 1274753, ThermoFisher).

Table 4 below shows an example of formulating a composition according to the stock solutions provided herein.

TABLE 4
Formulated Composition

| | | | | | Total | | | | | | |
|---|---|---|---|---|---|---|---|---|---|---|---|
| | | | | Combo | vol | | | | | | |
| | | | | # | (uL) | Sucrose | Glucose | Trehalose | Maltose | Melezitose | Histidine |
| Sucrose | glucose | histidine | glycerol | 3 | 1800 | 450 | 225 | | | | 225 |
| Sucrose | glucose | histidine | sorbital | 8 | 1800 | 450 | 225 | | | | 225 |
| Sucrose | trehalose | histidine | glycerol | 13 | 1800 | 450 | | 225 | | | 225 |
| Sucrose | trehalose | histidine | sorbitol | 18 | 1800 | 450 | | 225 | | | 225 |

| | | | | | | | | Theoretical | |
|---|---|---|---|---|---|---|---|---|---|
| | | | | | | | | | Final LNP |
| | | | | | | | LNP | LNP | conc. |
| | | | | Methionine | Glycerol | Sorbital | Vol | conc.(ug/mL) | (ug/mL) |
| Sucrose | glucose | histidine | glycerol | | 45 | | 855 | 202.7 | 96.2825 |
| Sucrose | glucose | histidine | sorbital | | | 45 | 855 | 202.7 | 96.2825 |
| Sucrose | trehalose | histidine | glycerol | | 45 | | 855 | 202.7 | 96.2825 |
| Sucrose | trehalose | histidine | sorbitol | | | 45 | 855 | 202.7 | 96.2825 |

### A Protocol is Provided as Follows.

A stock SAM-LNP buffer (20 mM Tris with 5 mM NaCl-NO SUCROSE) was made. Stock solutions as shown in Table 2 were obtained in the corresponding LNP buffer. Vial labels and Eppendorf tube labels were made. The gB SAM-LNP stock was thawed. Buffer exchange was performed using a PD10 column to remove sucrose according to a spin protocol (e.g., Spin Protocol of PD-10 Desalting Columns of Instructions 52-1308-00 BB; GE Healthcare). Formulations were prepared according to calculated volumes in Table 4. Two vials were filled for each formulation and placed on the tray.

The stock solution from Table 2 is mixed with buffer (Table 3) to make formulations shown in Table 4 to keep the final volume of the formulation consistent. In this case, the concentration of SAM-LNPs was kept unchanged after addition of all excipients.

### Example 2-Lower Drying Temperature Method

Two basic lyophilization methods (including some variations of the two methods) were used in the experiments reported below, a lower drying temperature method which is described in this Example 2 and a higher drying temperature method which is described in Example 3. Unless otherwise stated herein, the above-described procedures for preparing the LNPs and the formulation to be lyophilized were used in this Example.

Different compositions were evaluated after lyophilization and CQAs (e.g., size, PLI, % encapsulation) were measured and compared to a control formulation (e.g., a lyophilized control formulation and a pre-lyophilized control formulation) according to the techniques provided herein.

The lyophilization process was carried out according to the controlled parameters provided in Table 5 below (see, e.g., cycle time, step order, temperature, pressure as well as ramp rate). These parameters controlled the environment of the lyophilization chamber for each step.

### Lyophilization Process Conditions Summary

TABLE 5
Lower Drying Temperature Method Conditions for Sample
Loading, Thermal Equilibration and Freezing

| | Temp. | Time | Vacuum | Ramp | |
|---|---|---|---|---|---|
| Cycle Step | (° C.) | (hours) | (mTorr) | Rate | |
| Sample Loading | RT | — | — | — | |
| Thermal | 5 | 0.5 | — | 1° | C./min |
| Equilibration | −5 | 0.5 | — | 1° | C./min |
| Freezing | −40 | 1 | — | 1° | C./min |
| Primary Drying | −29 | 27 | 57 | 0.5° | C./min |
| Secondary Drying | 15 | 12 | 57 | 0.11° | C./min |

According to the above Table, a lyophilization process is shown according to the embodiments provided herein. A sample loading step is performed in which the lyophilization formulation, at room temperature, is loaded into the lyophilization chamber. A first thermal equilibrium cycle is performed with a ramp rate of 1° C. per minute to reach a temperature of 5° C. for a duration of 0.5 hours. Once completing the first step of thermal equilibrium, a second step of thermal equilibrium was performed. The second step of thermal equilibrium was performed with a ramp rate of 1° C. per minute to reach a temperature of minus 5° C. for a duration of 0.5 hours.

A freezing step, for a duration of 1 hour, was performed with a ramp rate of 1° C. per minute to reach a temperature of −40° C. Once completing the freezing step, a primary drying step was performed for a duration of 27 hours, with a ramp rate of 0.5° C. per minute to reach a temperature of minus 29° C. A secondary drying step followed the primary drying step. The secondary drying step was performed for a duration of 12 hours, with a ramp rate of 0.1° C. per minute to reach a temperature of 15° C.

The present lyophilization process when performed on a composition comprising gB SAM-LNP (conc. 202.7 μg/mL) in 20 mM Tris, 5 mM NaCl and 7.5% sucrose, pH 8.0 formed LNP particles with a size ranging from 110 to 130 nm, a PDI of 0.1-0.2 and a percent encapsulation decrease of 5-20% as compared to pre-lyophilization controls with a percent encapsulation of 85%, size of ˜120 nm, and PDI of about 0.1 (data not shown).

### Variations of Secondary Drying Time and Temperature

Table 6 below shows an alternative lyophilization process comprising a secondary drying step, wherein the temperature is about 5° C. for a drying time of about 48 hours.

TABLE 6
Alternative Lower Secondary Drying Temperature Process Conditions

| | Freezing rate of 1° C./min | | | | Freezing rate of 0.1° C./min | | | |
|---|---|---|---|---|---|---|---|---|
| | Temp | Time | Vacuum | Ramp | Temp | Time | Vacuum | Ramp |
| Cycle Step | (° C.) | (hours) | (mTorr) | Rate | (° C.) | (hours) | (mTorr) | Rate |
| Sample | RT | — | — | — | RT | — | — | — |
| Loading | | | | | | | | |
| Thermal | 5 | 0.5 | | 1° | 5 | 0.5 | — | 1° |
| Equilibration | | | | C./min | | | | C./min |
| | −5 | 0.5 | — | 1° | −5 | 0.5 | — | 1° |
| | | | | C./min | | | | C./min |
| Freezing | −40 | 1 | — | 1° | −40 | 1 | — | 0.1° |
| | | | | C./min | | | | C./min |
| Primary | −30 | 30 | 60 | 0.5° | −30 | 30 | 60 | 0.5° |
| Drying | | | | C./min | | | | C./min |
| Secondary | 5 | 48 | 60 | — | 5 | 48 | 60 | — |
| Drying | | | | | | | | |

Table 7 below shows an alternative lyophilization protocol in which the secondary drying time is extended. In Table 7, primary drying was performed according to Table 5 and then secondary drying conditions were changed. In general, a lower secondary drying temperature led to a longer drying duration time and a higher secondary drying temperature led to a shorter drying duration time. In this figure, higher drying temperatures led to a reduction in percent encapsulation of mRNA (post-lyo). Accordingly, in this experiment secondary drying temperatures at or below about 15° C. showed smaller reductions in percent encapsulation before and after lyophilization. The percent encapsulation was surprisingly preserved at about 80% with a secondary drying temperature of 5° C. for 48 hours, as compared to higher drying temperatures with shorter drying times that led to about 67-72% encapsulation.

TABLE 7
Alternative Lyophilization Protocol
with Extended Secondary Drying Time

| Secondary | Secondary | | % | % | % |
|---|---|---|---|---|---|
| Drying | Drying | | Entrapment | Entrapment | Entrapment |
| Temperature | Duration | | Pre-lyo | Post-lyo | Drop |
| 5° C. | 48 | hours | 92 | 81 | 11 |
| 15° C. | 12 | hours | 92 | 77 | 14 |
| 25° C. | 6 | hours | 84 | 67 | 17 |
| 40° C. | 4 | hours | 94 | 72 | 22 |

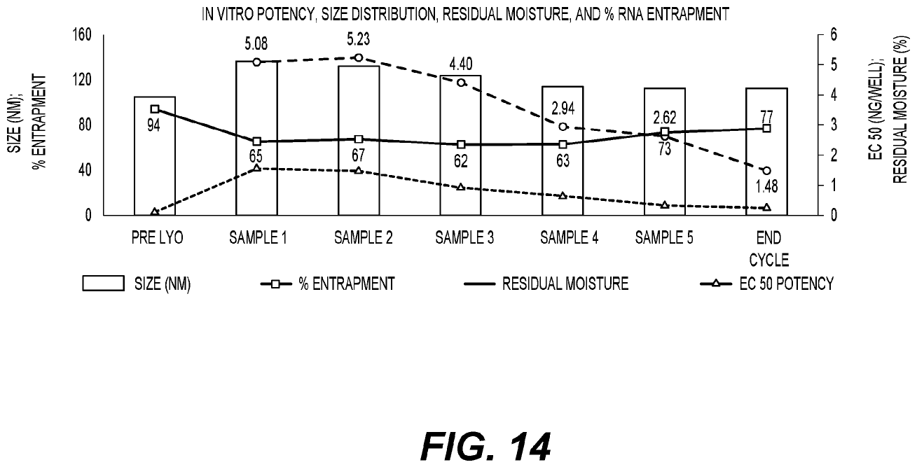

FIG. 14 shows data relating to the above-described alternative lyophilization process comprising a secondary drying temperature at 5° C. for 48 hours. A direct link between residual moisture and % RNA entrapment was not observed. During the secondary drying step, it was observed that the percent encapsulation initially decreased along with a larger particle size and reduced potency. However, as secondary drying continued, these characteristics trended toward their initial values. No significant changes in size distribution and RNA content were found.

During the lyophilization process, samples were withdrawn using a sample thief, starting from the end of primary drying through the end of lyophilization. For example, in some aspects, samples were removed every two hours and percent encapsulation tested.

### Observations on the Lower Drying Temperature Method

Surprisingly, it was found that different secondary drying temperatures and durations impacted the percentage entrapment post lyophilization. In some cases, selected combinations of secondary drying temperatures and time durations resulted in an improvement of percent encapsulation, as the percent encapsulation trended towards its initial value.

### Evaluation CQAs for Lyophilized Product

For both the process and formulation development of components and buffers, pre and post lyophilization samples were analyzed for size (e.g., using Dynamic Light Scattering (DLS) (Wyatt & Malvern assay) to determine hydrodynamic diameter and Polydispersity Index (PDI)), and percent encapsulation (e.g., using RiboGreen Fluorescence). RNA integrity was assessed by agarose gel electrophoresis. For select samples, lipid content was determined (by Reverse Phase HPLC) and moisture content (Karl Fisher).

### Particle Size-Determined by Dynamic Light Scattering

Liposome size can be determined in various ways (e.g., using the techniques of dynamic light scattering and/or single-particle optical sensing, using an apparatus such as the Accusizer™ and Nicomp™ series of instruments available from Particle Sizing Systems (Santa Barbara, USA), the Zetasizer™ instruments from Malvern Instruments (UK), or the Particle Size Distribution Analyzer instruments from Horiba (Kyoto, Japan)). See, Light Scattering from Polymer Solutions and Nanoparticle Dispersions Schartl, 2007. Dynamic light scattering (DLS) is the preferred method by which liposome size is determined. The preferred method for defining the average liposome diameter is a Z-average i.e. the intensity-weighted mean hydrodynamic size of the ensemble collection of liposomes measured by DLS. The Z-average is derived from cumulants analysis of the measured correlation curve, wherein a single particle size (droplet diameter) is assumed and a single exponential fit is applied to the autocorrelation function. Thus, references herein to average particle size should be taken as an intensity-weighted average, and ideally the Z-average. PDI values are easily provided by the same instrumentation which measures average diameter.

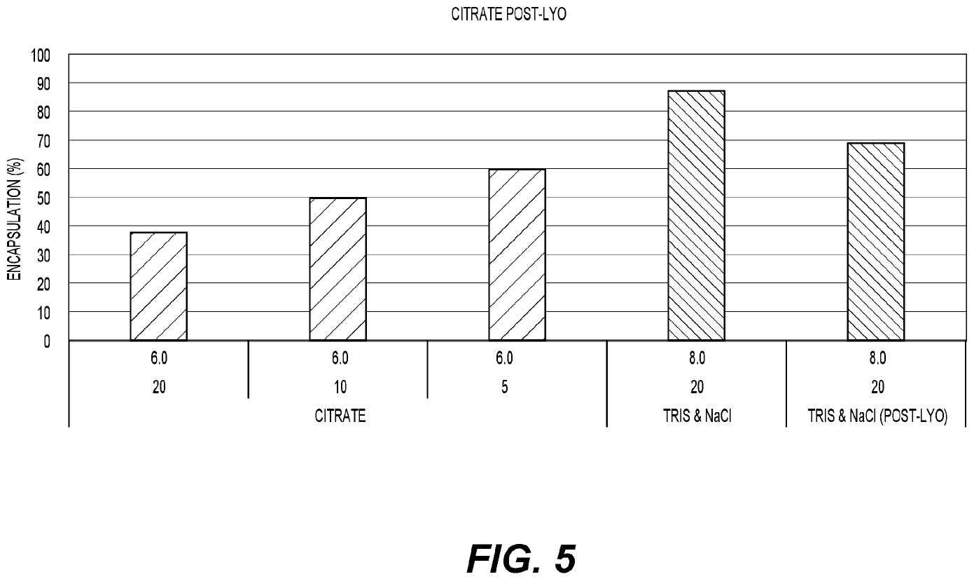

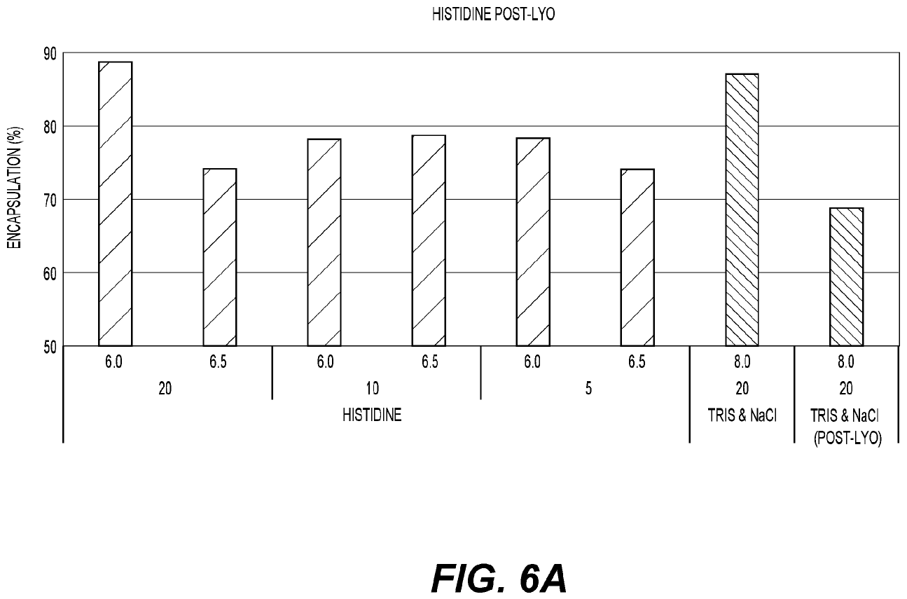

FIG. 6B shows sizes (nm) of reconstituted compositions comprising mRNA-LNP that underwent lyophilization. Various concentrations of histidine buffer are shown (e.g., 20 mM, 10 mM and 5 mM) at various pHs (6.0 and 6.5) as compared to a control composition (about 60-250 μg/mL SAM-LNP, 7.5% sucrose, 20 mM Tris, 5 mM NaCl, pH 8.0). Lyophilization conditions included a freezing step of about minus 40° C. for 1 hour, a primary drying step of about minus 29° C. for about 27 hours, and a secondary drying step of about 15° C. for about 12 hours.

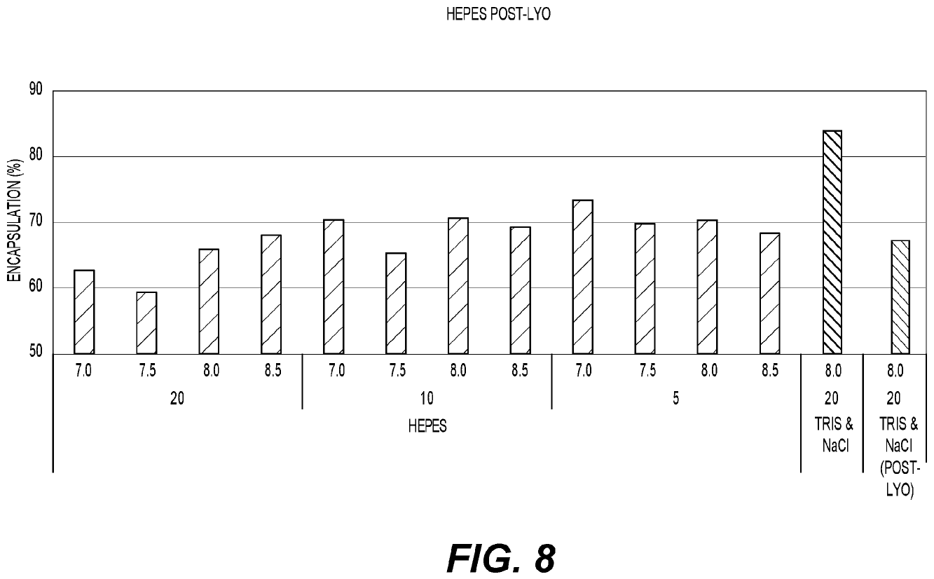

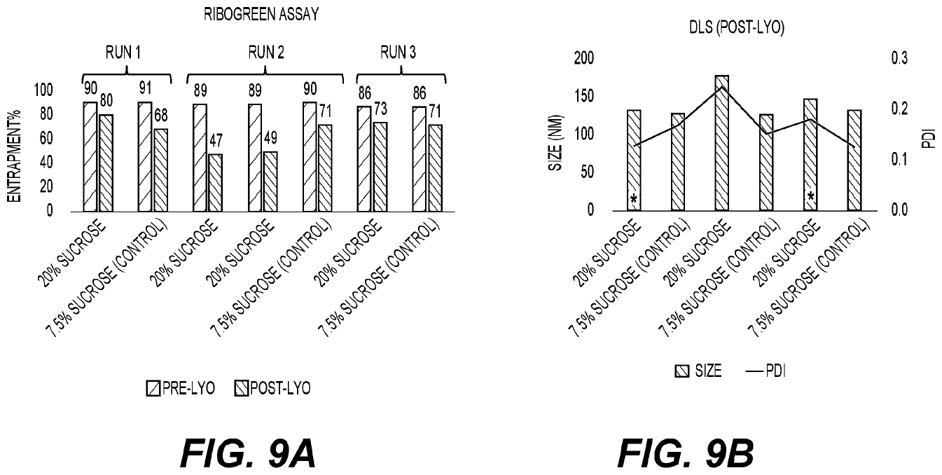

FIG. 9B shows the size and PDI of the reconstituted LNPs (the same LNPs as described in FIG. 9A) as the amount of sucrose was increased in the formulation. Lyophilization conditions included a freezing step of about minus 40° C. for about 1 hour, a primary drying step of about minus 29° C. for about 27 hours, and a secondary drying step of about 15° C. for about 12 hours.

Table 8 shows that compositions with increasing concentrations of sucrose did not lead to an increase in LNP particle size after lyophilization.

TABLE 8
Effect of Sucrose Concentration on LNP Particle Size

| | Size | PDI |
|---|---|---|
| Pre Lyo 7.5% | 121.6 | 0.071667 |
| Post Lyo 7.5% | 141.1667 | 0.123667 |
| Post Lyo 15% | 131.6333 | 0.090333 |
| Post Lyo 20% | 129.5667 | 0.087667 |
| Post Lyo 30% | 134.6667 | 0.110333 |

Lyophilization conditions included a freezing step of about minus 40° C. for about 1 hour, a primary drying step of about minus 29° C. for about 27 hours, and a secondary drying step of about 15° C. for about 12 hours.

### Determination of % Encapsulation

The percentage of encapsulated RNA and RNA concentration were determined by Quant-iT RiboGreen RNA reagent kit (Invitrogen), following manufacturer's instructions. The ribosomal

RNA standard provided in the kit was used to generate a standard curve. Liposomes were diluted 10x or 100x in 1X TE buffer (from kit) before addition of the dye. Separately, liposomes were diluted 10x or 100x in 1X TE buffer containing 0.5% Triton X before addition of the dye (to disrupt the liposomes and thus to assay total RNA). Thereafter an equal amount of dye was added to each solution and then ˜180 μL of each solution after dye addition was loaded in duplicate into a 96 well tissue culture plate. The fluorescence (Ex 485 nm, Em 528 nm) was read on a microplate reader. All liposome formulations were dosed in vivo based on the encapsulated amount of RNA.

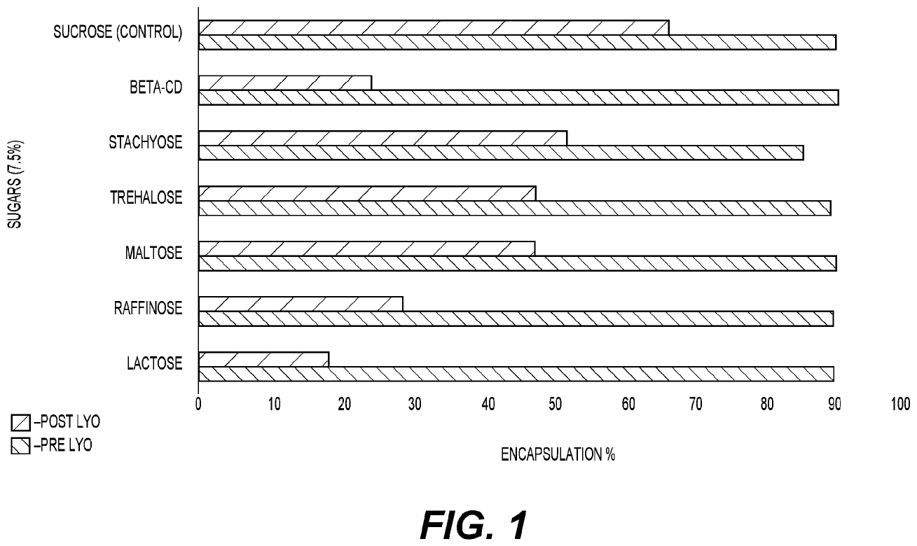

FIG. 1 shows percent encapsulation of various pharmaceutical compositions comprising different primary sugars at a concentration of 7.5% (w/v). These compositions were subjected to lyophilization and reconstituted, e.g., in sterile water. Percent encapsulation was determined by a RiboGreen fluorescence assay. In this example, the control composition (e.g., about 60-250 μg/mL SAM-LNP, 20 mM Tris, 5 mM NaCl, 7.5% sucrose, pH 8.0) was shown to have a higher percentage encapsulation of SAM-LNP after reconstitution as compared to pharmaceutical compositions comprising other sugars at 7.5% (w/v) (e.g., beta-CD, stachyose, trehalose, maltose, raffinose, and lactose). In this example, compositions comprising 7.5% (w/v) of sucrose showed about 65-70% encapsulation after reconstitution compared with about 90% encapsulation of the pre-lyophilized control. Substitution of sucrose with the other sugars (e.g., beta-CD, stachyose, trehalose, maltose, raffinose, and lactose) led to a further reduction in percent encapsulation as compared to the sucrose control. In general, the polydispersity index (PDI), a measure of size-based heterogeneity in nanoparticles, of the SAM-LNPs was less than 0.2 um (data not shown).

The liquid pharmaceutical composition was stored at minus 80° C. prior to lyophilization. Lyophilization conditions included a freezing step of about minus 40° C. for about 1 hour, a primary drying step of about minus 29° C. for about 27 hours, and a secondary drying step of about 15° C. for about 12 hours. In this example, the pharmaceutical composition that underwent lyophilization comprised about 60-250 μg/mL SAM-LNP, 20 mM Tris, 5 mM NaCl, 7.5% primary sugar (variable), and pH 8.0.

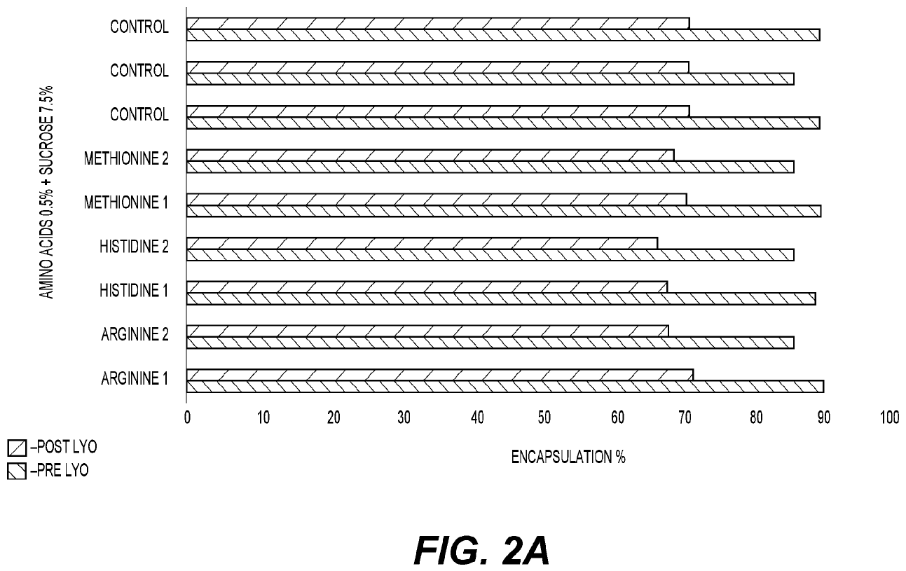

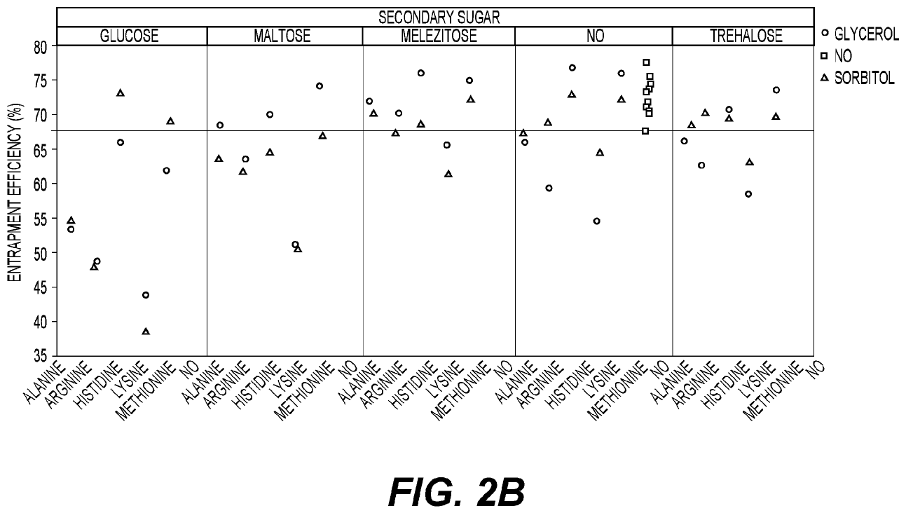

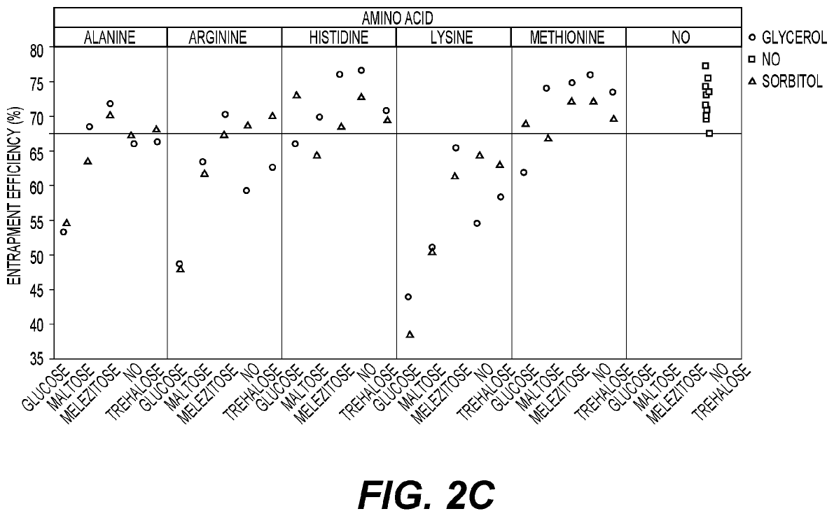

FIG. 2A shows percent encapsulation of various pharmaceutical compositions comprising sucrose at a concentration of about 7.5% (w/v) and amino acids at a concentration of about 0.5% (w/v). These compositions were subjected to lyophilization and reconstituted, e.g., in sterile water. As shown in this figure, reconstituted pharmaceutical compositions comprising methionine, histidine and arginine were shown to have a comparable percentage encapsulation of SAM-LNP as compared to the pre-lyophilized control (about 60-180 μg/mL SAM-LNP, 20 mM Tris, 5 mM NaCl, 7.5% sucrose, pH 8.0). In general, PDI was less than 0.2 um (data not shown).

In this example, the pharmaceutical composition subjected to lyophilization comprised; about 60-250 μg/mL SAM-LNP, 20 mM Tris, 5 mM NaCl, 7.5% primary sugar (variable), pH 8.0. The liquid pharmaceutical composition was stored at about minus 80° C. prior to lyophilization. Lyophilization conditions included a freezing step of about minus 40° C. for 1 hour, a primary drying step of about minus 29° C. for 27 hours, and a secondary drying step of about 15° C. for 12 hours.

FIG. 2B shows entrapment efficiency of various pharmaceutical compositions comprising sucrose at a concentration of about 5.0% (w/v), a secondary sugar at a concentration of about 2.5% (w/v), and various amino acids at a concentration of about 0.5% (w/v). These compositions were subjected to lyophilization and reconstituted, e.g., in sterile water. As shown, reconstituted pharmaceutical compositions comprising melezitose and trehalose were shown to have an improved percentage encapsulation (entrapment efficiency) of SAM-LNP as compared to the reconstituted compositions comprising glucose and maltose. These compositions included about 60-250 ug/m SAM-LNP, 20 mM Tris, 5 mM NaCl, 5.0% (w/v) sucrose, about 2.5% (w/v) secondary sugar (variable), about 0.5% (w/v) amino acid (variable), pH 8.0). Lyophilization conditions included a freezing step of about minus 40° C. for about 1 hour, a primary drying step of about minus 29° C. for about 27 hours, and a secondary drying step of about 15° C. for about 12 hours.

FIG. 2C shows entrapment efficiency of various pharmaceutical compositions comprising sucrose at a concentration of about 5.0% (w/v), a secondary sugar at a concentration of about 2.5% (w/v), and various amino acids at a concentration of about 0.5% (w/v). These compositions were subjected to lyophilization and reconstituted, e.g., in sterile water. As shown, reconstituted pharmaceutical compositions comprising histidine and methionine were shown to have an improved percentage encapsulation (entrapment efficiency) of SAM-LNP as compared to reconstituted compositions comprising alanine, arginine, or lysine. Compositions comprised about 60-250 μg/mL SAM-LNP, 20 mM Tris, 5 mM NaCl, 5.0% (w/v) sucrose, about 2.5% (w/v) secondary sugar (variable), and about 0.5% (w/v) amino acid (variable), pH 8.0). Lyophilization conditions included a freezing step of about minus 40° C. for about 1 hour, a primary drying step of about minus 29° C. for about 27 hours, and a secondary drying step of about 15° C. for about 12 hours.

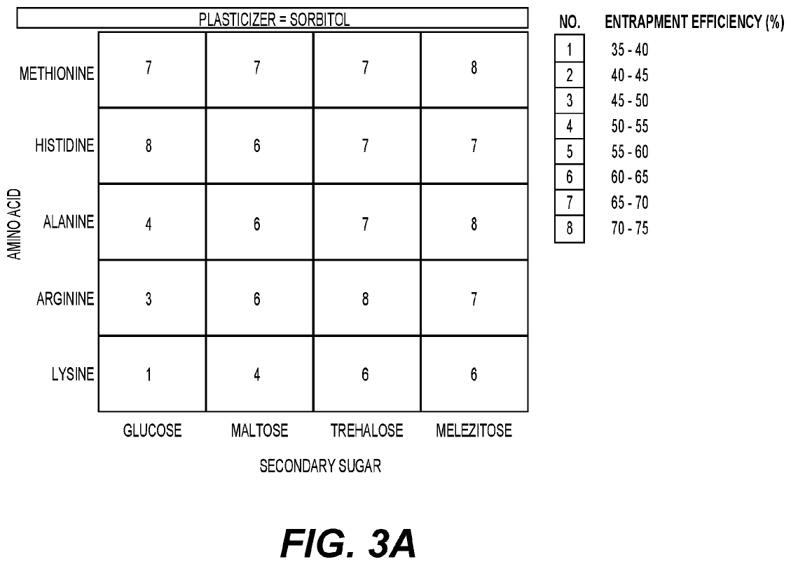

FIGS. 3A and 3B show heat maps for percentage encapsulation for various pharmaceutical compositions, which include a plasticizer of either sorbitol or glycerol, with amino acid(s) and secondary sugar(s) listed on different axes. All compositions contained a total of about 7.5% (w/v) of sugar, including about 5% (w/v) of sucrose and about 2.5% (w/v) of the secondary sugar. In general, reconstituted compositions comprising methionine and/or histidine in combination with trehalose and/or melezitose showed a higher percentage encapsulation as compared to other reconstituted combinations shown, with similar results for sorbitol and glycerol. Lyophilization conditions included a freezing step of about minus 40° C. for about 1 hour, a primary drying step of about minus 29° C. for about 27 hours, and a secondary drying step of about 15° C. for about 12 hours.

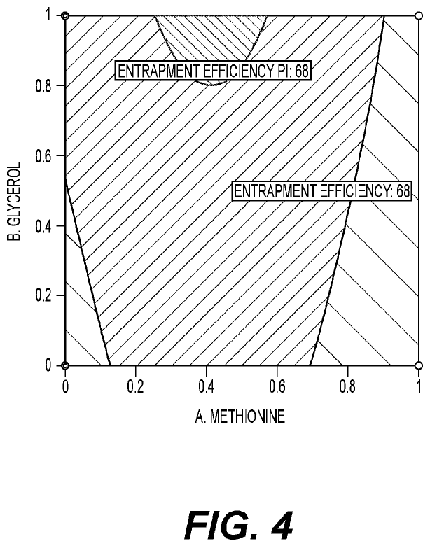

FIG. 5 shows percent encapsulation for compositions comprising about 60-250 μg/mL SAM-LNP and 7.5% (w/v) sucrose with varying concentrations of citrate buffer (e.g., 5 mM, 10 mM and 20 mM) at pH 6.0. Lyophilization conditions included a freezing step of about minus 40° C. for about 1 hour, a primary drying step of about minus 29° C. for about 27 hours, and a secondary drying step of about 15° C. for about 12 hours. Citrate buffer demonstrated low percent encapsulation after three freeze-thaw cycles. Reconstituted pharmaceutical compositions comprising citrate buffer demonstrated less than 60% encapsulation in comparison to a reconstituted control (post-lyo) and a frozen liquid control (about 87%) with about 60-250 μg/mL SAM-LNP. 7.5% sucrose, 20 mM Tris, 5 mM NaCl, at pH 8.0.

FIG. 6A shows percent encapsulation for compositions comprising about 60-250 μg/mL SAM-LNP and 7.5% (w/v) sucrose with varying concentrations of histidine buffer (e.g., 5 mM, 10 mM and 20 mM) at various pHs (e.g., 6.0 and 6.5). Lyophilization conditions included a freezing step of about minus 40° C. for about 1 hour, a primary drying step of about minus 29° C. for about 27 hours, and a secondary drying step of about 15° C. for about 12 hours. The reconstituted pharmaceutical composition (about 60-250 μg/mL SAM-LNP, 7.5% sucrose, 20 mM His, at pH 6.0) performed comparably to or better than the reconstituted control (about 60-250 ug/m SAM-LNP, 20 mM Tris 5, mM NaCl, 7.5% sucrose, pH 8.0) and a frozen liquid control that did not undergo lyophilization.

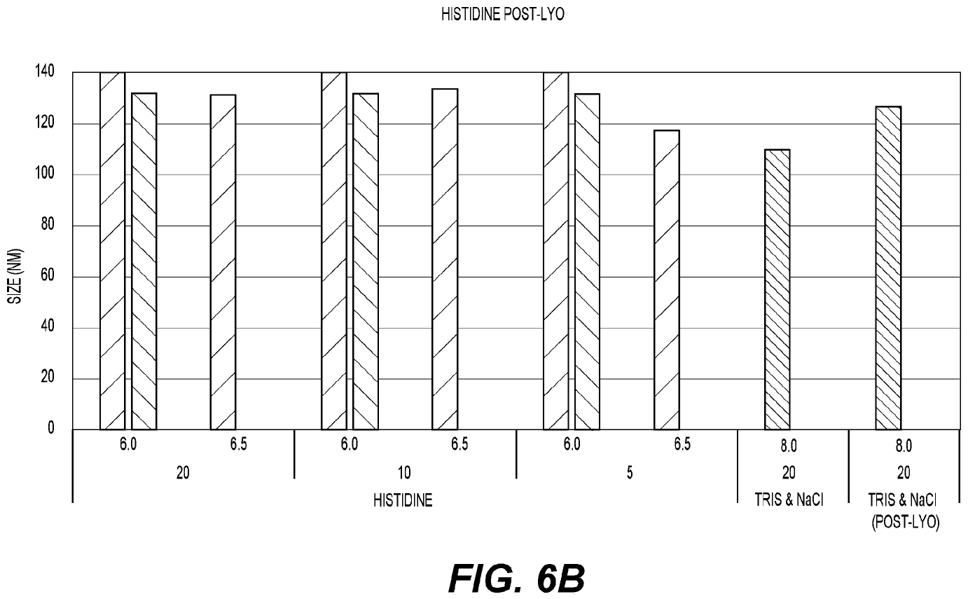

FIG. 7 shows percent encapsulation for compositions comprising about 60-250 μg/mL SAM-LNP and 7.5% (w/v) sucrose with varying concentrations of Tris buffer (5 mM, 10 mM, and 20 mM at various pHs ranging from 7.5 to 9.0). Lyophilization conditions included a freezing step of about minus 40° C. for about 1 hour, a primary drying step of about minus 29° C. for about 27 hours, and a secondary drying step of about 15° C. for about 12 hours. Compositions comprising Tris buffer were lyophilized. The reconstituted compositions demonstrated percent encapsulation that was comparable to the reconstituted control (about 60-250 μg/mL SAM-LNP, 20 mM Tris 5, mM NaCl, 7.5% sucrose, at pH 8.0). In particular, compositions comprising Tris buffer that were lyophilized and reconstituted were shown to be comparable to reconstituted compositions (about 70%) but less than a frozen liquid control (>85%).

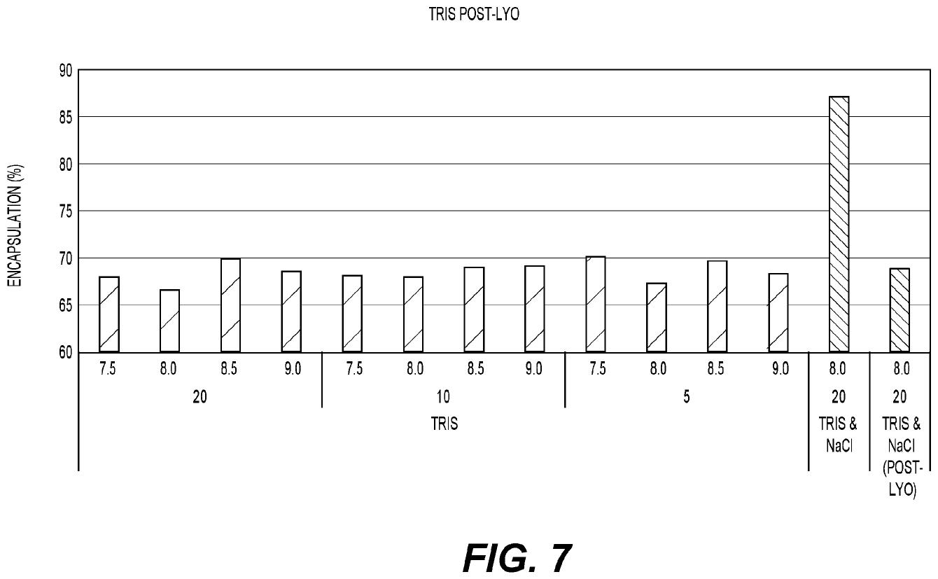

FIG. 8 shows percent encapsulation of compositions comprising HEPES buffer that underwent lyophilization. Compositions comprising HEPES buffer (e.g., 5 mM, 10 mM, and 20 mM at various pHs ranging from 7.0 to 8.5) demonstrated percent encapsulation (about 70%) that was comparable to the lyophilized control (about 60-250 μg/mL SAM-LNP, 20 mM HEPES, 7.5% sucrose). In particular, compositions comprising HEPES that were subjected to lyophilization were shown to have a percent encapsulation that was lower than a corresponding frozen liquid control (>85%) but similar to a reconstituted control. Lyophilization conditions included a freezing step of about minus 40° C. for about 1 hour, a primary drying step of about minus 29° C. for about 27 hours, and a secondary drying step of about 15° C. for about 12 hours.

FIG. 9A shows percent encapsulation after reconstitution of a lyophilized pharmaceutical composition comprising higher sucrose concentrations (up to about 20% sucrose). In some aspects, a higher amount of sucrose may improve percent encapsulation after lyophilization (scc, e.g., Run 1 and Run 3). Lyophilization conditions included a freezing step of about minus 40° C. for about 1 hour, a primary drying step of about minus 29° C. for about 27 hours, and a secondary drying step of about 15° C. for about 12 hours.

Table 9 below shows that increasing concentrations of sucrose did not affect percent encapsulation prior to lyophilization. This Table also shows the results of percent encapsulation after lyophilization for compositions with increasing amounts of sucrose.

TABLE 9
Effect of Sucrose Concentration on Percent
Encapsulation and Moisture Content

| Sample | Entrapment | Entrapment | Entrapment | Moisture |
|---|---|---|---|---|
| Pre Lyo | % Pre Lyo | % Post Lyo | % Drop | Content (KF) |
| 7.5% sucrose | 85.83 | 64.71 | 20 | 1.54 |
| 15% sucrose | 85.24 | 65.74 | 19 | 1.07 |
| 20% sucrose | 86.52 | 65.27 | 22 | 1.38 |
| 30% sucrose | 85.19 | 59.41 | 26 | 2.85 |

### Impact of Freezing Ramp Rate on LNPs

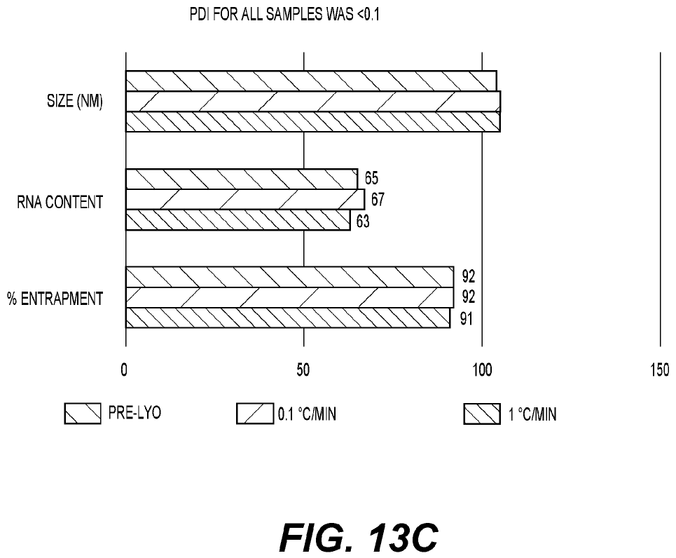

FIGS. 13A-13C show the impact of freezing ramp rate on the lipid nanoparticle.

FIG. 13A shows that the lipid content remained steady during the lyophilization process. In particular, the amount of each lipid present before lyophilization remained about the same as the amount of lipid after lyophilization over a ramp freezing rate ranging from about 0.1° C./min to about 1° C./min.

FIG. 13B shows that the LNP retain their in vitro potency over a ramp rate ranging from about 0.1° C./min to about 1° C./min. The in vitro potency (EC50) for the post-lyophilization samples was comparable with the pre-lyophilization samples, regardless of the freezing rate of 1° C./min or 0.1° C./min.

FIG. 13C shows the effects of particle size, percent encapsulation, and RNA content for LNPs over a ramp rate ranging from about 0.1° C./min to about 1° C./min. In particular, these parameters were shown to be relatively insensitive to ramp rate over this range and were comparable to the pre-lyophilization control. No change in size distribution or percent encapsulation was observed after freezing with a ramp rate of about 1° C./min or about 0.1° C./min.

### Effect of Excipients and Additives

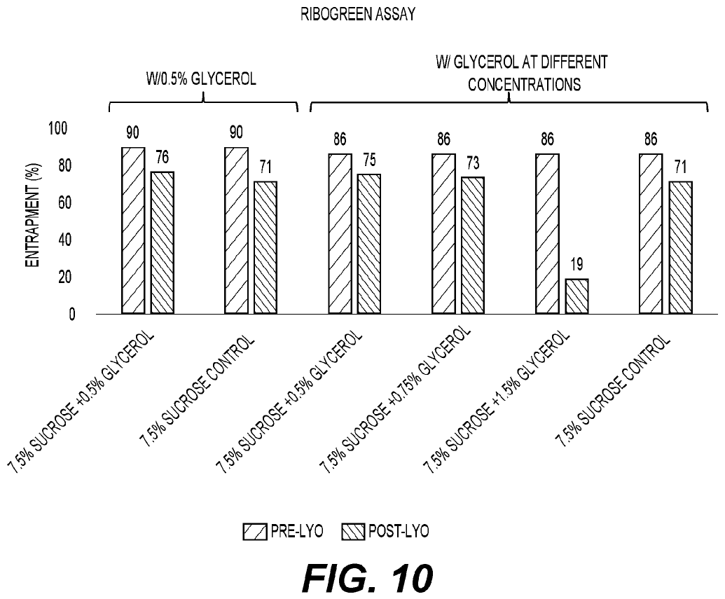

FIG. 10 shows that the addition of a plasticizer, in this case glycerol, improved the percentage entrapment of RNA-LNP after lyophilization. Compositions were formulated with amounts of sucrose and glycerol as indicated (about 60-250 μg/mL SAM-LNP, 7.5% (w/v) sucrose, 20 mM Tris, 5 mM NaCl, at pH 8.0). The lyophilized composition was reconstituted in sterile water and a RiboGreen assay was used to assess percent encapsulation. In particular, concentrations of glycerol of 0.5% (w/v) demonstrated the smallest decrease relative to the pre-lyophilized control. Lyophilization conditions included a freezing step of about minus 40° C. for about 1 hour, a primary drying step of about minus 29° C. for about 27 hours, and a secondary drying step of about 15° C. for about 12 hours.

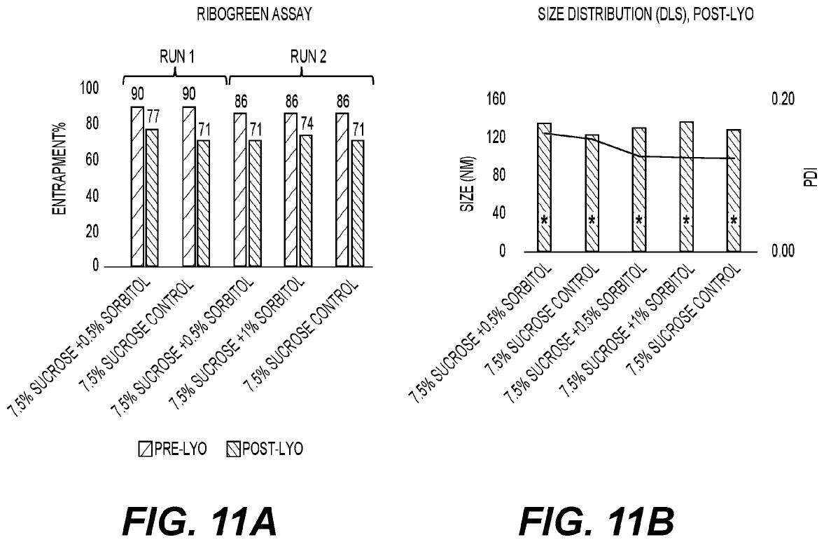

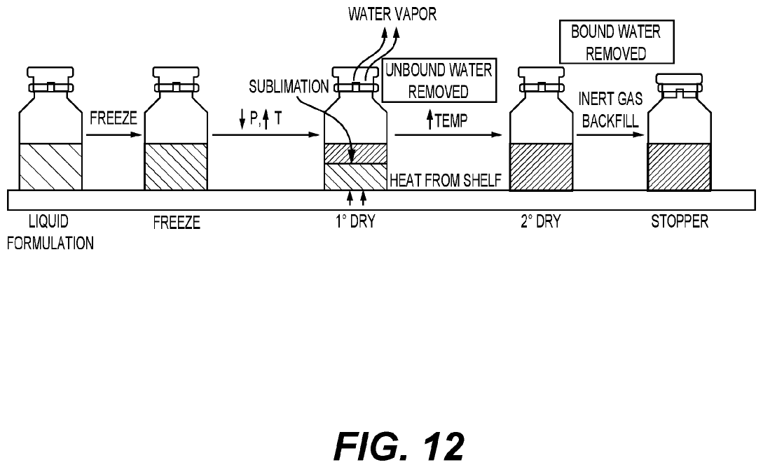

FIG. 11A shows that the addition of another plasticizer, in this case sorbitol, also improved the percentage entrapment of SAM-LNP after lyophilization. Compositions were formulated with amounts of sucrose and sorbitol as indicated (about 60-250 μg/mL SAM-LNP, 7.5% sucrose (w/v). 20 mM Tris, 5 mM NaCl, at pH 8.0). The lyophilized composition was reconstituted in sterile water and a RiboGreen assay was used to assess percent encapsulation. In particular, concentrations of sorbitol of 0.5% (w/v) (see, e.g., Run 1) demonstrated the smallest decrease relative to the pre-lyophilized control. Lyophilization conditions included a freezing step of minus 40° C. for about 1 hour, a primary drying step of minus 29° C. for about 27 hours, and a secondary drying step of about 15° C. for about 12 hours.

FIG. 11B shows that the size of the LNPs after lyophilization was not significantly impacted. Lyophilization conditions included a freezing step of about minus 40° C. for about 1 hour, a primary drying step of about minus 29° C. for about 27 hours, and a secondary drying step of about 15° C. for about 12 hours.

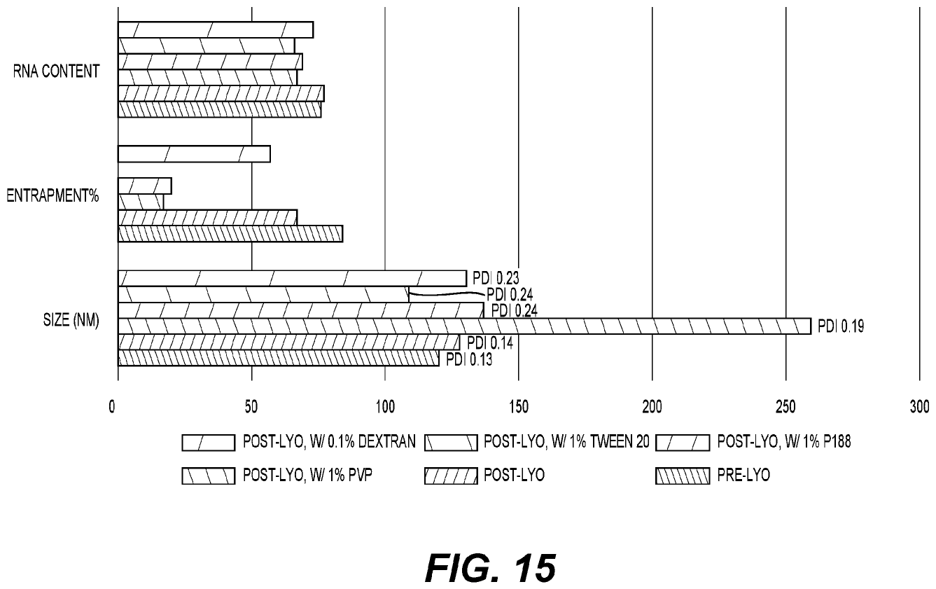

FIG. 15 shows various excipients added to the control formulation. Excipients included dextran, PVP, Tween 20 as well as P-188. Addition of these particular excipients, while not appearing to impact overall RNA content, generally led to a reduction in percent encapsulation (shown here as entrapment percentage) and in the case of PVP, led to larger particle sizes. The reduction in percent encapsulation and multimodal size distribution indicated that the presence of the excipients at these concentrations have a negative effect on the formation of the reconstituted RNA-LNP. Therefore, in some embodiments of the invention, these excipients and/or other excipients are not in the pharmaceutical compositions provided herein.

### Design of Experiments (DOE)

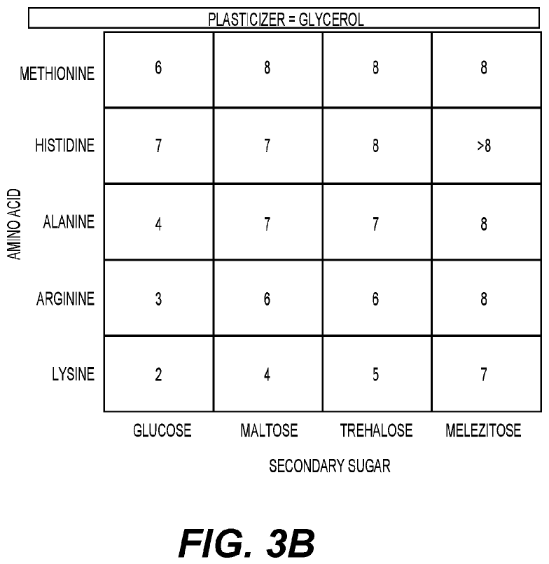

FIG. 4 shows a Design of Experiments (DoE) study with pharmaceutical compositions comprising SAM-LNP, characterized by an overall LNP size of 112 nm and a PDI of 0.135 with 89% encapsulation. This DoE investigated formulations comprising sucrose in combination with other ingredients such as a secondary sugar, an amino acid, and a plasticizer. This graph shows a predicted model of entrapment efficiency based on sucrose 10% (w/v), trehalose 0% (w/v), methionine, and glycerol. Optimal entrapment is predicted to be achieved with about 0.5% methionine and about 0.9% glycerol. In this study, secondary sugars were deemed not necessary to improve encapsulation. The results of the DoE also favored higher concentrations of sucrose and sufficient amounts of plasticizer and an amino acid to improve percent encapsulation during and after lyophilization.

A DoE study was performed to identify components that control the characteristics (e.g., size, PLI, % encapsulation) of the lyophilized compositions.

The above Table 10 shows that concentrations of histidine buffer of 5 mM histidine buffer or more (e.g., 5-30 mM histidine buffer) are comparable or improved as compared to a control (about 60-250 μg/mL SAM-LNP, 7.5% sucrose, 20 mM Tris and 5 mM NaCl, pH 8.0). Size (nm), percent encapsulation, PDI and pH are shown. An improvement in percent encapsulation is shown with 20 mM and 30 mM histidine buffer at about pH 6.0, with about a 15-20 nm increase in the size of the nanoparticles as compared to the reconstituted control. Lyophilization conditions included a freezing step of about minus 40° C. for about 1 hour, a primary drying step of about minus 29° C. for about 27 hours, and a secondary drying step of about 15° C. for about 12 hours.

### Buffer Variation

Table 10 below shows additional data for reconstituted compositions comprising variable concentrations of histidine buffer. A composition comprising about 60-250 μg/mL SAM-LNP and 7.5% sucrose in histidine buffer was lyophilized and reconstituted, e.g., in sterile filtered water.

TABLE 10
Additional Data at Various Concentrations of Histidine Buffer

| | Encapsulation | Size | | |
|---|---|---|---|---|
| Sample | (%) | (nm) | PDI | pH |
| 30 mM Histidine | 93.38 | 135.80 | 0.14 | 6.014 |
| Buffer SAM COVID | | | | |
| Lyo (TP 0) | | | | |
| 20 mM Histidine | 87.11 | 131.93 | 0.14 | 6.007 |
| Buffer SAM COVID | | | | |
| Lyo (TP 0) | | | | |
| 10 mM Histidine | 73.15 | 132.27 | 0.14 | 6.048 |
| Buffer SAM COVID | | | | |
| Lyo (TP 0) | | | | |
| 5 mM Histidine | 70.37 | 132.90 | 0.15 | 6.205 |
| Buffer SAM COVID | | | | |
| Lyo (TP 0) | | | | |
| 20 mM Tris Control | 75.86 | 115.40 | 0.10 | 8.077 |
| SAM COVID Lyo | | | | |
| (TP 0) | | | | |

SAM-LNPs were formulated in Tris buffer with 5 mM NaCl and 7.5% sucrose and concentrated as described above. For evaluation of compositions in various buffers, such as Tris, histidine, citrate, and HEPES buffer, LNPs in PBS were subjected to buffer exchange and/or desalting (e.g., such as by size exclusion chromatography) according to techniques known in the art. Any suitable protocol may be used (for example, Instructions 52-1308-00 BB from GE Healthcare (PD-10 Desalting Columns), with buffer exchange protocols).

Traditionally, it has been thought that acidic buffers such as citrate buffer or histidine buffer would not be suitable for lyophilization of LNP-mRNA due to decreased LNP stability. For example, for histidine buffer, which has a pKa of about 6.4, it was thought that the LNP would be less stable in acidic conditions.

With reference to FIGS. 6A, 6B and Table 10 discussed above, it was surprisingly found that lyophilization of the compositions provided herein in a 5 mM or greater histidine buffer provided an encapsulation percentage that was comparable or better than a liquid control that was not lyophilized. Histidine buffer performed better than others buffers that were evaluated. Surprisingly, histidine buffer also unexpectedly performed better than citrate buffer.

### In Vitro Potency

For select samples, in-vitro potency data was obtained (data not shown). It is desirable for the vaccine composition to have the same or very similar in vitro potency before lyophilization and after reconstitution (and after dilution, if dilution is performed). Any in vitro assay can be used to measure in vitro potency.

### In Vivo Immunogenicity

To assess in vivo expression of the RNA a reporter enzyme (SEAP; secreted alkaline phosphatase) was encoded in the replicon, rather than an immunogen. Expression levels were measured in sera diluted 1:4 in 1X Phospha-Light dilution buffer using a chemiluminescent alkaline phosphate substrate. 8-10 week old BALB/c mice (5/group) were injected intramuscularly on day 0,50 ul per leg with 0.1 ug or 1 μg RNA dose. The same vector was also administered without the liposomes (in RNase free 1X PBS) at 1 μg.

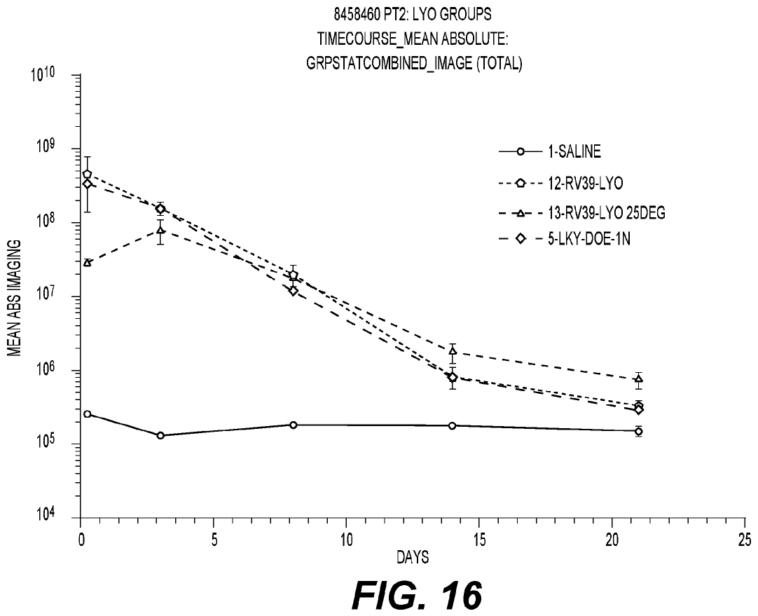

The following Table 11 and FIG. 16 show in vivo screening of compositions from the SAM-LNP DOE study.

TABLE 11
Vaccine Formulations

| Gr | N | Vaccine* | RNA Dose** | | Formulation |
|---|---|---|---|---|---|
| 1 | 2 | PBS | 50 | μL | — |
| 2 | 5 | SAM-Luc-F | 0.15 | μg | LKY-DOE-1K |
| 3 | 5 | SAM-Luc-F | 0.15 | μg | LKY-DOE-1L |
| 4 | 5 | SAM-Luc-F | 0.15 | μg | LKY-DOE-1M |
| 5 | 5 | SAM-Luc-F | 0.15 | μg | LKY-DOE-1N |
| 6 | 5 | SAM-Luc-F | 0.15 | μg | LKY-DOE-1O |
| 7 | 5 | SAM-Luc-F | 0.15 | μg | LKY-DOE-2A |
| 8 | 5 | SAM-Luc-F | 0.15 | μg | LKY-DOE-2B |
| 9 | 5 | SAM-Luc-F | 0.15 | μg | LKY-DOE-2C |
| 10 | 5 | SAM-Luc-F | 0.15 | μg | LKY-DOE-2D |
| 11 | 5 | SAM-Luc-F | 0.15 | μg | LKY-DOE-2E |
| 12 | 5 | Lyo SAM-Luc-F | 0.15 | μg | RV 39 |
| 13 | 5 | Lyo SAM-Luc-F 25 deg | 0.15 | μg | RV 39 |

For all LNP groups, a 50 intramuscular (i.m.) injection was given to 56 BALB/c female mice, 7-8 weeks old, wherein the dose was adminstered to each of the two hind legs (25 ul per quadriceps). Serum was collected on the day of administration (D) and at 3 weeks post administration (D21). Luminescence and F-specific neutralizing Ab titers were measured as readouts of immunogenicity.

Images were obtained 6 h post injection of SAM-LNP and at 3, 8, 14 and 21 days post injection. The terminal bleed was performed on day 21. In all cases, the N: P ratio for all LNP was 8:1.

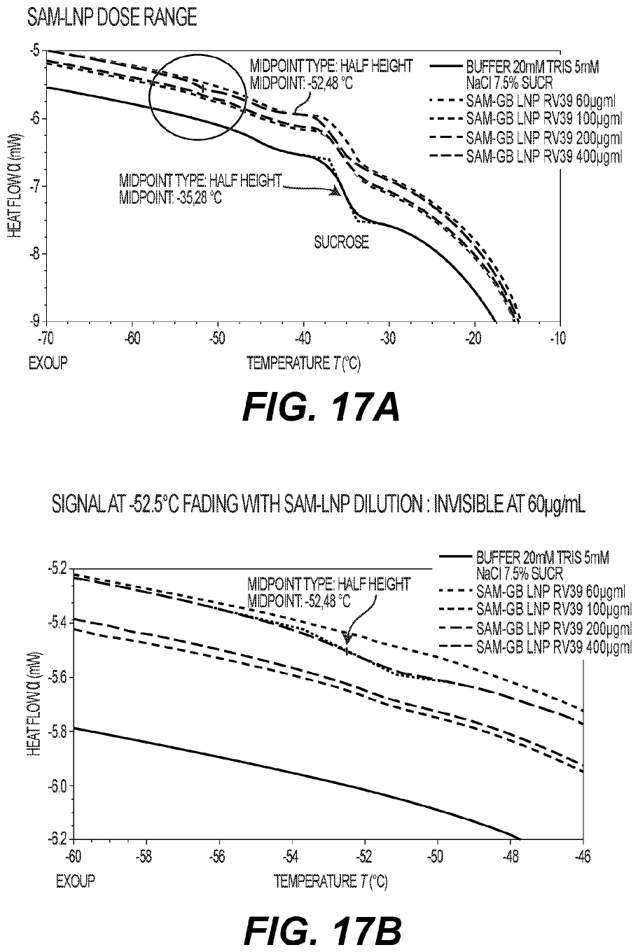

In this study, various formulations with SAM-LNP including RV39 (cationic lipid, 2,5-bis((9Z,12Z)-octadeca-9,12-dien-1-yloxy)benzyl 4-(dimethylamino) butanoate) were investigated. Bioluminescence of samples taken at the referenced time points show results in FIG. 17A and FIG. 17B with corresponding formulations shown in FIGS. 18A-18C. Formulations are provided in FIG. 17A.

FIG. 16 shows in vivo screening of the SAM-LNP DOE study indicating non-inferiority between frozen liquid and lyophilized samples. In an in vivo study in mice, the bioluminescence for luciferase SAM-LNP for liquid and lyophilized product (stored at 5° C. and 25° C.) was compared, and demonstrated a comparable luminescence profile in vivo to liquid SAM-LNP.

Compositions are provided Tables 12, 13 and 14 below provide a listing of compositions. Each of the compositions followed a similar trend. A saline control was included as well.

TABLE 12

| Theoretical | | | | | | |
|---|---|---|---|---|---|---|
| conc. | Form. | | | | Methionine/ | |
| (μg/mL) | # | Pattern | Sucrose | Trehalose | Histidine | Glycerol |
| 80.8 | *1 | 0 | 7.5 | 2.5 | 0.5 | 0.5 |
| 19.0 | *2 | +−++ | 10 | 5 | 1 | 0 |
| 85.5 | 3 | 0a00 | 7.5 | 2.5 | 0.5 | 0 |
| 80.8 | 4 | 0 | 7.5 | 2.5 | 0.5 | 0.5 |
| 80.8 | 5 | 0 | 7.5 | 2.5 | 0.5 | 0.5 |
| 104.5 | 6 | −−+− | 5 | 5 | 0 | 0 |
| 80.8 | 7 | 0 | 7.5 | 2.5 | 0.5 | 0.5 |
| 80.8 | 8 | 0 | 7.5 | 2.5 | 0.5 | 0.5 |
| 104.5 | *9 | 00a0 | 7.5 | 0 | 0.5 | 0.5 |
| 95.0 | 10 | ++−− | 5 | 0 | 1 | 1 |
| 104.5 | 11 | a000 | 7.5 | 2.5 | 0 | 0.5 |
| 61.8 | 12 | 000A | 10 | 2.5 | 0.5 | 0.5 |
| 104.5 | 13 | −+−+ | 10 | 0 | 0 | 1 |
| 95.0 | 14 | −++− | 5 | 5 | 0 | 1 |
| 80.8 | 15 | 0 | 7.5 | 2.5 | 0.5 | 0.5 |
| 57.0 | *16 | −+++ | 10 | 5 | 0 | 1 |
| 66.5 | *17 | −−++ | 10 | 5 | 0 | 0 |
| 142.5 | 18 | −+−− | 5 | 0 | 0 | 1 |
| 152.0 | 19 | −−−− | 5 | 0 | 0 | 0 |
| 47.5 | *20 | +++− | 5 | 5 | 1 | 1 |
| 57.0 | *21 | 00A0 | 7.5 | 5 | 0.5 | 0.5 |
| 80.8 | 22 | 0 | 7.5 | 2.5 | 0.5 | 0.5 |
| 57.0 | 23 | +−+− | 5 | 5 | 1 | 0 |
| 104.5 | 24 | +−−− | 5 | 0 | 1 | 0 |
| 9.5 | *25 | ++++ | 10 | 5 | 1 | 1 |
| 57.0 | 26 | A000 | 7.5 | 2.5 | 1 | 0.5 |
| 80.8 | 27 | 0 | 7.5 | 2.5 | 0.5 | 0.5 |
| 114.0 | 28 | −−−+ | 10 | 0 | 0 | 0 |
| 80.8 | 29 | 0 | 7.5 | 2.5 | 0.5 | 0.5 |
| 80.8 | 30 | 0 | 7.5 | 2.5 | 0.5 | 0.5 |
| 57.0 | *31 | ++−+ | 10 | 0 | 1 | 1 |
| 99.8 | 32 | 000a | 5 | 2.5 | 0.5 | 0.5 |
| 66.5 | 33 | +−−+ | 10 | 0 | 1 | 0 |
| 76.0 | *34 | 0A00 | 7.5 | 2.5 | 0.5 | 1 |

It should be noted that the Form #s (Formulation Numbers) marked with an asterisk (*) in Table 12 correspond to the respective DOE numbers in Tables 13 and 14. For example, DOE-1 refers to Form. #1 in Table 12.

TABLE 13

| | | | | | Theoretical | Current | Current |
|---|---|---|---|---|---|---|---|
| | | | | | Conc. | Cycle - | cycle - DoE |
| Sample | Sucrose | Trehalose | Methionine | Glycerol | (ug/mL) | repeat | (run 1) |
| DOE-1 | 7.5 | 2.5 | 0.5 | 0.5 | 80.75 | 117.83 | 119.00 |
| DOE-2 | 10 | 5 | 1 | 0 | 19 | 120.53 | 119.00 |
| DOE-9 | 7.5 | 0 | 0.5 | 0.5 | 104.5 | 118.87 | 118.50 |
| DOE-12 | 10 | 2.5 | 0.5 | 0.5 | 61.75 | 114.10 | 130.40 |
| DOE-16 | 10 | 5 | 0 | 1 | 57 | 114.17 | 118.80 |
| DOE-17 | 10 | 5 | 0 | 0 | 66.5 | 115.10 | 117.80 |
| DOE-20 | 5 | 5 | 1 | 1 | 47.5 | 136.30 | 143.60 |
| DOE-21 | 7.5 | 5 | 0.5 | 0.5 | 57 | 116.60 | 120.00 |
| DOE-25 | 10 | 5 | 1 | 1 | 9.5 | 110.83 | 132.60 |
| DOE-31 | 10 | 0 | 1 | 1 | 57 | 140.93 | 137.73 |
| DOE-34 | 7.5 | 2.5 | 0.5 | 1 | 76 | 126.83 | 123.43 |
| water | 7.5 | 0 | 0 | 0 | 133 | 123.00 | 122.8 |
| (7.5% suc) | | | | | | | |
| Control 1 | 7.5 | 0 | 0 | 0 | 190 | 119.80 | 119.1 |
| Control 2 | 7.5 | 0 | 0 | 0 | 133 | 116.70 | 117.3 |

TABLE 14

| | | | | | Theoretical | Current | Current |
|---|---|---|---|---|---|---|---|
| | | | | | Conc. | Cycle - | cycle - DoE |
| Sample | Sucrose | Trehalose | Methionine | Glycerol | (ug/mL) | repeat | (run 1) |
| DOE-1 | 7.5 | 2.5 | 0.5 | 0.5 | 80.75 | 63.47 | 68.98 |
| DOE-2 | 10 | 5 | 1 | 0 | 19 | 54.66 | 60.70 |
| DOE-9 | 7.5 | 0 | 0.5 | 0.5 | 104.5 | 67.17 | 71.99 |
| DOE-12 | 10 | 2.5 | 0.5 | 0.5 | 61.75 | 65.17 | 70.78 |
| DOE-16 | 10 | 5 | 0 | 1 | 57 | 62.90 | 68.33 |
| DOE-17 | 10 | 5 | 0 | 0 | 66.5 | 59.11 | 65.08 |
| DOE-20 | 5 | 5 | 1 | 1 | 47.5 | 54.50 | 59.61 |
| DOE-21 | 7.5 | 5 | 0.5 | 0.5 | 57 | 65.78 | 66.14 |
| DOE-25 | 10 | 5 | 1 | 1 | 9.5 | 44.68 | 64.18 |
| DOE-31 | 10 | 0 | 1 | 1 | 57 | 58.00 | 70.58 |
| DOE-34 | 7.5 | 2.5 | 0.5 | 1 | 76 | 60.39 | 69.86 |
| water | 7.5 | 0 | 0 | 0 | 133 | 67.40 | 64.31 |
| (7.5% suc) | | | | | | | |
| Control 1 | 7.5 | 0 | 0 | 0 | 190 | 64.90 | 68.49 |
| Control 2 | 7.5 | 0 | 0 | 0 | 133 | 62.33 | 61.47 |
| liq. | 7.5 | 0 | 0 | 0 | 190 | 88.50 | 88.80 |
| control | | | | | | | |

It was observed that in vitro potency for both pre- and post-lyophilized SAM-LNP were similar despite a decrease in percent encapsulation. The reconstituted composition maintained or at least improved the product CQAs post lyophilization as compared to the lyophilized control.

### Example 3-Higher Drying-Temperature Process

Unless otherwise stated herein, the above-described procedures for preparing the formulation to be lyophilized as well as the lyophilization procedures described Example 2 were used in this Example.

An alternative higher drying-temperature method (an alternative to the lower drying method described in Example 2) was conducted under the same conditions reported in the lower-drying temperature method of Example 2 above, except for the changes noted below.

This higher drying temperature method was attempted because of a small energy transition that was observed by differential scanning calorimetry (that is indicative of a release of energy) at −52.5° C. Freezing to below any observed transition temperature prevents molecular mobility leading to potential degradation of the RNA containing LNPs during primary drying. This transition was not noticed in initial studies (at lower concentrations of SAM/LNP in samples), but the energy transition was noticed when the concentration of SAM-LNPs in samples that were lyophilized was increased to 400 μg/ml. The transition is noticeable in the DSC analysis graph shown in FIG. 17B. The sample used in this analysis (400 μg/mL of SAM-LNP) was frozen to −119° C. at a temperature drop of 1° C./min. The DSC analysis graph was generated using this frozen sample and heating the sample and at a heating rate of 10° C./min over a temperature range of −120° C. to 20° C.

FIG. 17B, which is an exploded view of a portion of FIG. 17A. In view of this observed energy transition, an alternative lyophilization process was attempted wherein the freezing temperature was reduced to a temperature below the observed energy transition. In the experiment reported in FIGS. 17A and 17B, freezing to a temperature of −119° C. at a ramp rate of 1° C./min. Freezing to a temperature lower than the transition temperature is desirable as an energy transition is indicative of molecular movement within the LNP formulation which could possibly cause a loss of product quality. Therefore, freezing of the product at a temperature less than the observed energy transition temperature is desirable to maintain product quality.

The product that was frozen to a temperature less than the energy transition temperature was also subject to an aggressive (higher temperature) primary drying step which has an added benefit of shortening the primary drying temperature and expose the product over this transition temperature for a shorter time than for the low temperature cycle.

Lyophilization of SAM-LNPs using such a higher drying temperature method was carried out according to the controlled parameters provided in Table 15 below (see, e.g., cycle time, step order, temperature, pressure as well as ramp rate). These parameters controlled the environment of the lyophilization chamber for each step.

TABLE 15
Higher Drying Temperature Method Conditions

| | T° | Vacuum | Ramp | Duration |
|---|---|---|---|---|
| | (° C.) | (μbar) | (° C./min) | (h) |
| Sample loading | RT | — | — | — |
| Thermal | 5 | — | 1 | 0.5 |
| equilibration | −5 | — | 1 | 0.5 |
| Freezing | −65 | — | 1 | 1 |
| Primary | ≥0 | 30 | As fast as possible, | According to |
| Drying | | | depending upon | Pirani* and |
| | | | freeze-dryer | Tp (Temperature |
| | | | performance | of product)** |
| Secondary | 15 | 30 | 0.1 | 12 |
| Drying | | | | |

*A Pirani probe is a passive PAT tool that can be used to monitor the water vapor in the drying chamber of the lyophilizer. The Pirani pressure is sensitive to the water vapor present in the drying chamber. When the chamber is fully saturated with water vapor (i.e. during sublimation), the Pirani pressure is up to 1.6 times higher than the chamber pressure (monitored by a capacitive manometer). At the end of sublimation, the Pirani pressure starts to decrease until it reaches the value of the chamber pressure indicating that sublimation is finished.

**Product temperature is measured with wireless probes inside vials filled with product. When the product temperature matches the freeze-dryer shelves temperature, the sublimation (endothermic) is finished, meaning the primary drying is finished.

According to Table 15, a lyophilization process is shown according to the higher drying temperature embodiments provided herein. A sample loading step is performed in which the lyophilization formulation, at room temperature, is loaded into the lyophilization chamber. A first thermal equilibrium cycle is performed with a ramp rate of 1° C. per minute to reach a temperature of 5° C. for a duration of 0.5 hours. Once completing the first step of thermal equilibrium, a second step of thermal equilibrium was performed. The second step of thermal equilibrium was performed with a ramp rate of 1° C. per minute to reach a temperature of minus 5° C. for a duration of 0.5 hours.

A freezing step, for a duration of 1 hour was performed with a ramp rate of 1° C. per minute to reach a temperature of −65° C.

Once the freezing step is completed, a primary drying step is performed. The duration of this process was based on the in-process chamber pressure value given by the Pirani probe and the product temperature measured by temperature probes placed inside vials filled with product. Accordingly, the following drying cycles were performed. For the 0° C. cycle; primary drying was 15 hours, the 10° C.; cycle primary drying was 13.5 hrs, and for the 15° C. cycle based on the Pirani probe, primary drying was 9 hours. Accordingly, the ramp rate for each primary drying cycle was 1.1° C./min for the 0° C. cycle and 1.3° C./min for the 10 and 15° C., respectively.

The secondary drying step was then performed for a duration of 12 hours, with a ramp rate of 0.1° C. per minute to reach a temperature of 15° C.

The higher drying temperature method reported in Table 15 can be completed in less than 30 hours. More specifically, the total duration of the aforementioned cycles were the following: 0° C.; duration of 29 hours, 10° C.; duration of 27.5 hours, and 15° C.; a duration of 17 hours, respectively.

Table 16 below presents particle size and PDI results of RNA containing LNPs (in particular SAM-LNPs) at the three aforementioned different primary drying temperatures, all being at or above 0° C. (0° C., 10° C. and 15° C.).

TABLE 16
Particle Size and PDI

| Primary drying Tshelf | | | |
|---|---|---|---|
| (Shelf temperature) | | | |
| Characteristic | 0° C. | 10° C. | 15° C. |
| Av Particles size after | 128 nm/+3 nm | 127 nm/+7 nm | 130 nm/+0 nm |
| FD (nm)/variation | | | |
| compared to FB | | | |
| Av PdI after FD/ | 0.18/+0 | 0.16/+0.01 | 0.16/−0.08 |
| variation compared | | | |
| to FB | | | |

Further details concerning generation of the data summarized in Table 16 above are summarized in Tables 17, 18 and 19 below:

TABLE 17
Primary Drying at 0° C.

| | T° | Vacuum | Ramp | Duration |
|---|---|---|---|---|
| | (° C.) | (μbar) | (° C./min) | (h) |
| Sample loading | RT | — | — | — |
| Thermal | 5 | — | 1 | 0.5 |
| equilibration | −5 | — | 1 | 0.5 |
| Freezing | −65 | — | 1 | 1 |
| Primary Drying | 0 | 30 | 1.1 | 15 |
| Secondary Drying | 15 | 30 | 0.1 | 12 |

TABLE 18
Primary Drying at 10° C.

| | T° | Vacuum | Ramp | Duration |
|---|---|---|---|---|
| | (° C.) | (μbar) | (° C./min) | (h) |
| Sample loading | RT | — | — | — |
| Thermal | 5 | — | 1 | 0.5 |
| equilibration | −5 | — | 1 | 0.5 |
| Freezing | −65 | — | 1 | 1 |
| Primary Drying | 10 | 30 | 1.3 | 13.5 |
| Secondary Drying | 15 | 30 | 0.1 | 12 |

TABLE 19
Primary Drying at 15° C.

| | T° | Vacuum | Ramp | Duration |
|---|---|---|---|---|
| | (° C.) | (μbar) | (° C./min) | (h) |
| Sample loading | RT | — | — | — |
| Thermal | 5 | — | 1 | 0.5 |
| equilibration | −5 | — | 1 | 0.5 |
| Freezing | −65 | — | 1 | 1 |
| Primary Drying and | 15 | 30 | 1.3 | 15 |
| secondary drying* | | | | |

TABLE 20
Comparison of Properties of RNA Containing LNPs for the Lower Drying
Temperature Method and the Higher Drying Temperature Method:

| Cycle | | | | Lower Drying Temperature Method | | |
|---|---|---|---|---|---|---|
| Characteristics | 15° C. | | | (conditions from Table 5 above) | | |
| SAM-LNP | N68172-9-C | N68170-29 | | N68172-9-C | N68170-29 | |
| concentrated | | | | | | |
| batch | | | | | | |
| % Residual | 1.89 | 1.17 | 1.35 | 0.30 | 0.67 | 0.42 |
| Moisture | | | | | | |
| Av Particles size | 130 nm/+0 nm | 134 nm/+10 nm | 134 nm/+11 nm | 125 nm/+5 nm | 127 nm/+3 nm | 127 nm/+4 nm |
| after FD (nm)/ | | | | | | |
| variation | | | | | | |
| compared to FB | | | | | | |
| Av PdI after FD/ | 0.16/−0.08 | 0.13/+0 | 0.13/+0 | 0.16/−0.01 | 0.15/+0.02 | 0.13/+0 |
| variation | | | | | | |
| compared to FB | | | | | | |
| Tg onset (° C.) | ND | 36.9 | 37.7 | 41.1 | 35.6 | 36.7 |
| % Entrapment/ | 77%/−6% | 63%/−10% | 62%/−9% | 72%/−13% | 58%/−15% | 58%/−13% |
| variation | | | | | | |
| compared to FB | | | | | | |

Based on the data reported in the above experiments, including the above Tables, ideal temperatures for the higher drying temperature process would include temperatures for the primary drying step of between 0° C. and 20° C. When considering balancing interests of product quality (taking into consideration the above-measured characteristics) and speed of lyophilization, a useful drying temperature for the primary drying step in the higher drying temperature process would be greater than or equal to 0° C. and less than or equal to 15° C. Preferred temperatures of the primary drying step of the higher drying temperature process could be greater than 0° C. but below 20° C.

It will be understood that the invention has been described by way of example only and modifications may be made whilst remaining within the scope and spirit of the invention.

1. A pharmaceutical composition formulated for stable lyophilization of lipid nanoparticle encapsulated RNA comprising:
lipid nanoparticle encapsulated RNA (RNA-LNPs) at a concentration ranging from about 60 μg/mL to 250 μg/mL, wherein the lipid nanoparticle comprises a cationic lipid, a zwitterionic lipid, a cholesterol, and a PEG, and
wherein the pharmaceutical composition comprises:
sucrose in an amount of at least 5% (w/v); and
a buffer.

2. The pharmaceutical composition of claim 1, wherein said RNA is mRNA and said RNA-LNPs are mRNA-containing lipid nanoparticles (mRNA-LNPs).

3. The pharmaceutical composition of claim 1, wherein said RNA is self-amplifying mRNA (SAM) and said RNA-LNPs are SAM-containing lipid nanoparticles (SAM-LNPs).

4. The pharmaceutical composition of claim 1, wherein the cationic lipid is RV39 and the zwitterionic lipid is DSPC.

5. The pharmaceutical composition of claim 1,
wherein the cationic lipid is DlinDMA and the zwitterionic lipid is DSPC.

6. The pharmaceutical composition of claim 1, wherein sucrose is present in an amount of 7.5% (w/v), and wherein the buffer is Tris buffer with NaCl.

7. The pharmaceutical composition of claim 6, further comprising one or more of:
a plasticizer selected from the group consisting of sorbitol or glycerol in an amount of 0.25%-1.0% (w/v), and preferably about 0.5% (w/v); and
an amino acid selected from the group consisting of methionine, histidine, and arginine in an amount of 0.25%-1.0% (w/v), and preferably about 0.5% (w/v).

8. The pharmaceutical composition of claim 1, wherein sucrose is present in an amount of 7.5% (w/v), and wherein the buffer is histidine buffer.

9. The pharmaceutical composition of claim 8, wherein histidine buffer is 20 to 30 mM histidine buffer and the pH is from 6 to 6.5.

10. The pharmaceutical composition of claim 8, additionally comprising one or more of:
a plasticizer selected from the group consisting of sorbitol or glycerol in an amount of 0.25%-1.0% (w/v), and preferably about 0.5% (w/v); and
an amino acid selected from the group consisting of methionine, histidine, and arginine in an amount of 0.25%-1.0% (w/v), and preferably about 0.5% (w/v).

11. The pharmaceutical composition of claim 1, wherein sucrose is present in an amount of 20% (w/v), and wherein the buffer is a histidine buffer or a Tris buffer.

12. The pharmaceutical composition of claim 1, wherein the composition additionally comprises:
a secondary sugar selected from the group consisting of trehalose, glucose, stachyose, or maltose present in an amount of about 2.5% (w/v),
wherein sucrose is present in an amount of 5.0% (w/v); and
wherein the buffer comprises a Tris buffer with NaCl or a histidine buffer.

13. The pharmaceutical composition of claim 12, additionally comprising one or more of:
a plasticizer selected from the group consisting of sorbitol or glycerol in an amount of 0.25%-1.0% (w/v), and preferably about 0.5% (w/v); and
an amino acid selected from the group consisting of methionine, histidine, and arginine in an amount of 0.25%-1.0% (w/v), and preferably about 0.5% (w/v).

14. A vaccine comprising the pharmaceutical composition of claim 1, wherein the pharmaceutical composition is lyophilized.

15. A kit comprising the lyophilized vaccine of claim 14, wherein the kit includes:
the lyophilized vaccine in a container;
a sterile needle for injecting a vaccine composition; and/or
a second container containing a sterile aqueous solution and/or an adjuvant.

16. A method of lyophilization of the pharmaceutical composition of claim 1 comprising:
placing the composition into a lyophilization chamber;
subjecting the composition to a freezing step that comprises decreasing the temperature of the lyophilization chamber from an initial temperature to a freezing temperature of about-39 or lower, at a controlled freezing ramp rate and holding the chamber at the freezing temperature to convert water to ice.

17. The method of lyophilization of claim 16, where the freezing temperature is between −39-° C. to −80° C.

18. The method of lyophilization of claim 16, where the controlled freezing ramp rate is from about 0.1° C./min to 2.0° C./min.

19. The method of lyophilization of claim 16, where the composition is held at the freezing temperature for 30 minutes or more.

20. A method of lyophilization of the pharmaceutical composition of claim 1 comprising:
placing the composition into a lyophilization chamber;
subjecting the composition to an initial freezing step that comprises decreasing the temperature of the lyophilization chamber to a freezing temperature of about-39 or −40° C., with a freezing ramp rate ranging from about 0.1° C./min to 1.0° C./min, and holding the chamber at the freezing temperature for one hour or more.

21. The method of lyophilization of claim 16 further comprising:
subjecting, after the initial freezing step, the composition to a primary drying step comprising raising the temperature of the lyophilization chamber to a primary drying temperature ranging from −25° C. to −35° C. with a ramp rate ranging from 0.1° C./min up to 1.0° C./min; and
maintaining the chamber at the primary drying temperature for 25 or more hours at a pressure of about 57-60 m Torr.

22. The method of lyophilization of claim 17 further comprising:
subjecting, after the primary drying step, the composition to a secondary drying step comprising raising the temperature of the lyophilization chamber to a secondary drying temperature ranging from 0° C. to 40° C. with a ramp rate ranging from 0.1° C./min up to 1.0° C./min, wherein the chamber is held at a pressure of about 57-60 mTorr.

23. The method of lyophilization of claim 18 wherein the secondary drying temperature is about 5° C. for 48 hours.

24. The method of lyophilization of claim 18 wherein the secondary drying temperature is about 15° C. for about 12 hours.

25. The method of lyophilization of claim 16, further comprising:
a first thermal equilibrium cycle with a ramp rate of 1° C. per minute to reach a temperature of 5° C. for a duration 0.5 hours; and
a second thermal equilibrium cycle with a ramp rate of 1° C. per minute to reach a temperature of −5° C. for a duration of 0.5 hours.

26. A reconstituted pharmaceutical composition of claim 14, wherein the percent entrapment of the reconstituted pharmaceutical composition is within 20% of the pharmaceutical composition prior to lyophilization.

27. A reconstituted pharmaceutical composition of claim 14, wherein the size of the reconstituted pharmaceutical composition is within a range of 110-130 nm.

28. A reconstituted pharmaceutical composition of claim 14, wherein the polydispersity index (PDI) of the reconstituted pharmaceutical composition is about 0.1-0.2.

29. A reconstituted pharmaceutical composition of claim 14, wherein the efficacy of the reconstituted pharmaceutical composition is within 10 percent of the efficacy of the pharmaceutical composition prior to lyophilization.

30. A method of lyophilization of claim 16, comprising:
placing the composition into a lyophilization chamber;
subjecting the composition to a freezing step that comprises decreasing the temperature of the lyophilization chamber to a freezing temperature of below −52.5° C., at a controlled freezing ramp rate and to convert water to ice and form a frozen composition.

31. The method of lyophilization of claim 16 comprising:
placing the composition into a lyophilization chamber;
subjecting the composition to a freezing step that comprises decreasing the temperature of the lyophilization chamber to a freezing temperature of below −40° C., at a controlled freezing ramp and to convert water to ice to form a frozen composition; and
raising the temperature of the lyophilization chamber during a primary drying step to a drying temperature of greater than 0° C. at a reduced pressure and at a controlled ramp rate to cause sublimation of ice to a gas.

32. The method of lyophilization of claim 31, wherein the primary drying step is conducted for a period of time less than or equal to 30 hours.

33. The pharmaceutical composition of claim 1 for use in inducing an acceptable immune response in a subject, where the pharmaceutical composition is administered by injection.

34. A method of preparing the reconstituted pharmaceutical composition of claim 26 for use in inducing an acceptable immune response in a subject, comprising reconstituting said lyophilized composition in a form suitable to be administered by injection.
